(19) United States
(12) Patent Application Publication
(10) Pub. No.: US 2026/0199547 A1
(43) Pub. Date: Jul. 16, 2026

(54) FLOWABLE CHITOSAN BIOADHESIVE HEMOSTATIC COMPOSITIONS THAT RESIST DISSOLUTION

(71) Applicant: TRICOL BIOMEDICAL, INC., Portland, OR (US)

(72) Inventors: MacKayla CAROLAN, Portland, OR (US); Madison BUTLER, Portland, OR (US); Grace RIEMAN, Portland, OR (US); Angel BUI, Portland, OR (US); Shalini GAUTAM, Portland, OR (US); Simon J. MCCARTHY, Portland, OR (US)

(21) Appl. No.: 19/135,693

(22) PCT Filed: Dec. 5, 2023
(86) PCT No.: PCT/US2023/082568

§ 371 Date: Jun. 4, 2025

Related U.S. Application Data
(60) Provisional application No. 63/386,312, filed on Dec. 6, 2022.
(60) Provisional application No. 63/386,313, filed on Dec. 6, 2022.
(60) Provisional application No. 63/386,314, filed on Dec. 6, 2022.

(51) Int. Cl. A61L 26/00; C08L 5/08

(52) U.S. Cl. CPC A61L 26/0095; A61L 26/0023; C08L 5/08; A61L 2400/04; A61L 2400/06

(57) ABSTRACT

The present invention relates to a biocompatible, tissue adhesive, chitosan flowable dressing, optionally modified with catechol, and suitable for treating bleeding in a physiological environment, e.g., gastrointestinal tract, bladder (in particular in connection with the TURP procedure). The characteristics and structures of the chitosan dressing are provided. Methods of making and using the chitosan dressing are also provided.

### BACKGROUND

### Technical Field

This disclosure relates to fluidized, flowable, liquid, hemostatic dispersions (alternately particle dispersions, dispersed particles, suspensions, suspended particles, or particle suspensions) of chitosan materials. The dispersions of the disclosure provide ability to accurately deliver inside the body safe and effective controlled amounts of tissue adherent, dissolution-resistant chitosan materials by minimally invasive techniques. The flowable chitosan materials of the invention are able to quickly adhere to mucosa and different tissue surfaces to stay in place and remain adhered to persist and provide prolonged hemostatic, protective, antibacterial and wound healing efficacy under inhospitable, internal, biological wet tissue environments where presently no other flowable device or material has demonstrated similar persistence and efficacy.

Flowable, liquid hemostats for use in the body, not relying on a hemostatic mechanical compression mode of action, are limited to two types of hemostats: (1) tissue-sealant hemostats; and (2) dispersed particle hemostats (alternately dispersed hemostats or liquid dispersed hemostats).

Flowable tissue-sealant hemostats constitute a continuous liquid adhesive composition (>98% w/w) that is intended to directly adhere to an injury site and promote closing of a wound by a non-biological mechanical process. Tissue sealant hemostats are generally contraindicated for direct application onto actively bleeding wounds. Because tissue sealants are continuous phase adhesive materials without porosity, their mode of action is readily compromised by the presence of liquids including blood which interfere with the adhesion process. Tissue-sealant hemostats are not effective in causing blood to clot because they present a low specific surface area to blood. Tissue-sealant hemostats can fail rapidly if they do not achieve complete sealing of an injury.

In contrast, flowable, liquid, dispersed, particle hemostats are composed of a continuous diluent carrier fluid (<95% w/w) phase and a dispersed discrete (particle) phase (≥5% w/w) with a paste-like, but flowable consistency. Dispersed particle hemostats being composed of a low viscosity diluent and dispersed solid or semi-solid particles ideally have an interconnected porous structure that provides for the hemostat to be able to present a high specific surface area to blood.

There is little information in the scientific and patent literature on flowable liquid dispersed particle hemostats other than they are most often composed of micron sized, solid, semi-solid cross-linked particles of gelatin dispersed at ≥5% w/w in carrier water or saline solution. Pro-clotting, biologically active thrombin is added to the gelatin particle dispersion to provide for a high specific surface area of rapidly pro-clotting gelatin particles. SURGIFLO (Ethicon) and FLOSEAL (Baxter) are commercially available examples of thrombin-based biologic flowable liquid dispersed hemostats. Biologic flowable liquid dispersed hemostats are those that include one or more active therapeutic biologically derived agents that promote blood clot formation by an active therapeutic chemical pathway such as, for example, thrombin or fibrinogen. SURGIFLO and FLOSEAL are mixed homogeneously into their liquid dispersions at point of care in 5 ml or 10 ml sizes by mixing of the dry powder ingredient with the carrier liquid and thrombin in two connected syringes. In the biologic flowable liquid dispersed hemostats, flow of blood between the particles of the hemostat results in high specific surface area of blood contact with the particles and fast clot formation in the biologic drug rich environment. As a substantially passive, hydrophilic material, the cross-linked gelatin particles of the biologic fluidized particle hemostat provide for mechanical support, partially swelling in the presence of fluid diluent, and adsorption of the biologically active hemostatic drug. Biologic liquid dispersed hemostats may be applied directly to actively bleeding wounds to effect fast acute control of bleeding. Some concerns with use of biologic liquid dispersed hemostats include: (i) their hemostatic efficacy is dependent on the use of the biologic drug thrombin or other biologic drugs including fibrinogen which are expensive, have limited shelf-life, and whose use is known to result in immunological reactions in some patients; (ii) biologic liquid dispersed hemostats cannot be effectively applied to control bleeding in circumstances where any prolonged tissue adherence (>6 hrs) to a surface is necessary such as upside-down, in the gastrointestinal tract, in the bladder and in a resected prostate; iii) adverse scar formation has been reported with the use of biologic liquid dispersed hemostats. A non-biologic, tissue adherent, fluid-type, liquid dispersed hemostat, for direct application to actively bleeding wounds and for prolonged application (>6 hrs) in general and difficult environments to control moderate to robust bleeding is not addressed in the literature or by currently available hemostatic products. The compositions and methods described herein seek to address these deficiencies.

Preparation of non-biologic, liquid dispersed material compositions which rely for their mode of action on tissue adhesion and safe, reliable, and effective delivery of bioactive material within the body remains a major problem. Non-biologic compositions do not include one or more active therapeutic biologically derived agents that promote blood clot formation by an active therapeutic chemical pathway such as, for example, thrombin or fibrinogen. In use, the non-biologic, liquid dispersed hemostatic material are intended to remain intact for a period of at least 12 hours in the presence of biological fluids such as gastric fluid, blood, bile and urine and are intended be able to remain adhered to tissue including mucosa of the gastrointestinal tract and the internal elastic lamina of the vascular system for a period longer than 12 hours while promoting localized hemostasis and wound healing with reduced risk of scarring. The non-biologic liquid dispersed hemostatic materials of the invention that meet these demanding requirements are dispersions of particles in the radius of gyration range of 10 to 350 microns. The dispersed particles of the invention are formed of compositions that include bioactive chitosan materials that are locally hemostatic without risk of remote emboli or thrombi and that promote normal healing without risk of scarring.

Bioactive chitosan has many applications in the body not limited to drug delivery, hemostasis, wound healing, tissue regeneration, and transarterial embolotherapy. A “bioactive” material is one that has activity that is not a drug or therapeutic agent activity and is therefore not subject to the same regulation as an active pharmaceutical or biologic therapeutic agent. Bioactivity may be related to interactions of insoluble material surfaces (typically of polymeric materials) with cellular environments and produces a measurable change or modification to the cellular environment. Current chitosan materials and compositions of chitosan material used on, or within the body are limited in their application as they are unable to resist dissolution and rapid loss in harsh environments such as the stomach and bladder, and they cannot be maintained in place by their adhesion to tissue. As an example of this limitation in chitosan materials, Table 4 in Subramanian et al. 2022 [1] details absence of chitosan-based materials being employed commercially for therapeutic applications requiring mucoadhesion and dissolution resistance in the stomach. Other than the delivery of last resort styptic solutions to control general and local bleeding, there are no recognized hemostatic therapies for use in the bladder and urological tract. Prolonged oozing bleeding that remains the most frequent adverse event following transurethral resection of the prostate (TURP) still relies on the use of a painful 1959 Foley balloon catheter traction with application up to 72 hours.

Liquid dispersion hemostatic material delivery has advantages over other modes of delivery such as a gas mode of delivery. Unlike liquid carried particle dispersions, gas carried particle dispersions are necessarily dry on delivery, and have numerous delivery problems including i) risk of tissue damage from the velocity and momentum of the pressurized stream; ii) blocking of the delivery catheter lumen end if wetted; iii) undesirable gas expansion in the body on delivery; iv) prolonged loss of visualization of the injury site from suspended particle haze; iv) inability to deliver the divergent nebulized particle stream locally to an injury site; v) up to 100× the required dose of hemostatic agent due to poor targeting and loss of visualization; vi) delivery of nebulized gas-borne particles to undesired locations such as the articulated joints of an endoscopic which can subsequently seize and which may make removal of an endoscope in retroflex position a serious adverse event; and vi) absence of immediate adhesion/cohesion due to dryness of delivered particles (dry particles need to be wet before they adhere together and attach to the wound surface). This dryness and initial poor adhesion results in migration of locally delivered material away from the target area further exacerbating the problem of adequate delivery of hemostatic agent.

This disclosure relates to the field of flowable, liquid, dispersed, dissolution-resistant, tissue adherent, persistent adherent, chitosan hemostatic materials able to be applied effectively in difficult wet tissue environments. Difficult wet tissue environments are typified by the highly digestive wet environment of the stomach and the urine saturated environment of the bladder and the urethra. The flowable chitosan material systems and methods of the invention preferably comprises catechol modified chitosan and uses thereof.

### Description of the Related Art

There are many surgeries on vascularized organs and tissue including, but not limited to, the heart, liver, pancreas, stomach, intestine, colon, prostate, tonsils, ear, nose, throat and brain that often continue to bleed following injury and through wound healing. The initial injury may continue to bleed for days unless standard hemostasis is applied and the bleeding may also recur at a later time. Standard of care initial hemostasis for treatment of bleeding following surgery is varied depending on the type of surgery. Ligature, gauze packing, biologic dressings, cautery, and banding may be applied to locally address the issue. Packing may be applied for up to 24 hours in the case of protracted bleeding. If the bleeding is uncontrollable after the period of conservational management, the patient may have to return to surgical unit promptly to stop the hemorrhaging with either open or endoscopic procedures. Although there have been advances in bleeding control using advanced dressings none of these advances have yet translated into reliable treatment options under unique surgical conditions where delivery, tissue adhesion, and continuous bleeding intermingled with other biological fluid considerations are highly challenging. Rapid bleeding control under all circumstances is highly desirable.

Minimally invasive surgical procedures are becoming the preferred means of interventional access due to ability to access areas of the body with significantly reduced risk (compared to open surgery) with lower morbidity, lower hospital cost and lower patient discomfort. Advanced biomaterials are at the forefront of addressing current limitations and enabling improvements in safety, reliability and increased practice and application of minimally invasive surgical procedures. Current limitations in minimally invasive surgical practice include control of bleeding (especially hemorrhage), sutureless closure of delicate soft tissue sites, local promotion of healing, local treatment of pathogenic conditions, and local placement with anchoring or adhesion to a surgical/interventional site. More recently minimally invasive interventions are being used to address vascular pathogenesis including vascular malformations, aneurysms, and vascular tumors. A preferred procedure to address vascular malformations, aneurysms, and vascular tumors is the use of transarterial embolotherapy to occlude abnormal blood vessels. Transarterial embolotherapy involves the local delivery of an occlusion device such as a stent or a coil of material or an anchored biomaterial which remain in place over an extended time to promote the formation of a localized clot with subsequent permanent closure of the vessel.

A biocompatible, tissue adhesive, chitosan material, locally applied and delivered, that remains intact for a prolonged time with promotion of local clot formation without risk of remote emboli, and normal tissue healing with reduced risk of scarring presents advanced material attributes that will enable significant development in surgical practice.

In addition, prolonged bleeding, with its associated risks in mortality and morbidity, remains a serious problem in the gastrointestinal (GI) tract. Techniques and devices that could provide for rapid bleeding control in gastrointestinal bleeding (GIB) for both upper gastrointestinal bleeding (UGIB) and lower gastrointestinal bleeding (LGIB) are needed. Current bleeding control in and after transurethral resection of the prostate (TURP) relies on cautery for small vessel arterial bleeding and application of balloon pressure to address venous oozing. The bladder neck and prostrate are both highly vascularized tissue that often continue to bleed following injury and through wound healing. The initial injury site may continue to bleed for days unless standard hemostasis is applied and the bleeding may also recur around week one or week two after TURP procedure when the scab of prostatic cavity sheds off. Current standard initial hemostasis for treatment of bleeding following TURP is to apply manual traction with a balloon catheter followed by continuous bladder irrigation with saline. Typically, balloon pressure can be applied for up to 24 hours in the case of protracted bleeding. If the bleeding is uncontrollable after the period of conservational management, the patient may have to return to surgical unit promptly to stop the hemorrhaging with either open or endoscopic procedures.

Although there have been advances in bleeding control using advanced dressings for applications outside of GIB or TURP bleeding control, none of these advances using chitosan materials [1] have yet translated to the unique conditions of the gastrointestinal tract or bladder and especially the upper gastrointestinal tract and prostrate where delivery, adhesion, enzyme activity, continuous oozing bleeding, acidity, and urine-related considerations are highly challenging. Rapid bleeding control in TURP, in open prostatectomy and in bladder resection is highly desirable.

Gastrointestinal bleeding (GIB) is a common presentation to the emergency department. According to the U.S. Department of Health and Human Service, from 2000 to 2014, there was an average of over 350,000 discharges from gastrointestinal hemorrhage annually. In the U.S., the direct hospital cost in 2010 due to GIB exceeded $1.1 billion [2]. Upper GIB (UGIB), defined as gastrointestinal bleeding proximal to the ligament of Treitz, is approximately five times more common than lower GIB (LGIB) [3]. Acute UGIB is a potentially life-threatening emergency that necessitates prompt assessment, resuscitation and appropriate medical and endoscopic management. Despite recent advances in management of GIB in western countries, the mortality rate of acute UGIB has not significantly improved, and remains as high as 10-14% [4, 5]. The major cause of death after GIB is death secondary to cardiorespiratory complications, which is not surprising given the burden of comorbidities in such patients; death due to uncontrollable hemorrhage is reported to account for between 20% and 25% of cases [6, 7]. While little can be done to correct comorbidities urgently, more effective and rapid bleeding control will allow significant reductions in the incidence of UGIB related morbidity and mortality. In general, the most common causes of acute UGIB are peptic ulcers, gastro-esophageal varices, Mallory-Weiss tears and erosive esophagogastritis [8]. Nonvariceal upper gastrointestinal bleeding (NVUGIB) encompasses all causes of UGIB except bleeding esophageal or gastric varices. The incidence of peptic ulcer disease has decreased because of the development and utilization of proton pump inhibitors as well as the identification, treatment and eradication of Helicobacter pylori in individual patients [9]. Despite decreased peptide ulcer incidence, mortality among NVUGIB patients ranges from 3-4% [10]. While rarely life threatening, gastric malignancies can lead to friable tissue with diffuse bleeding that is difficult to address with traditional physical hemostatic methods (clips, bands, ligation) or cautery [11].

Current endoscopic management of patients with acute UGIB includes thermal therapy (e.g., bipolar electrocoagulation, heater probe, monopolar electrocoagulation, argon plasma coagulation, and laser), injection (epinephrine, sclerosants (e.g., absolute ethanol, polidocanol, and ethanolamine)), thrombin or fibrin glue (thrombin plus fibrinogen)), and clips [12, 13].

In general, the majority of patients with bleeding peptic ulcers, hemostasis is achieved with combination of the above endoscopic therapeutic modalities. However, there remains a subset of patients, approximately 5%, in which endoscopic treatments are not sufficient for hemostasis and thus require interventional radiology or surgical interventions [14, 15].

Endoscopic therapy fails for a variety of reasons including poor visibility of lesion due to active pulsating bleeding, difficult anatomic location of lesion for endoscopy, maximal therapy with currently available tools, and severe coagulopathy. Three different gas propelled, gas dispersed, spray-based, hemostatic powder devices ENDOCLOT [16], HEMOSPRAY [17-21] and NEXPOWDER [18, 22, 23] have been developed to assist in the control of NVUGIB. HEMOSPRAY, cleared for sale in the US in 2018, although demonstrating improved ability to control acute upper gastrointestinal hemorrhage has been unable to improve incidence of rebleeding [20]. In the case of moderate to high flow hemorrhagic bleeding, poorly cohesive liquids or particles are easily flushed away from the wound. This problem of delivering sufficient loose hemostatic material in close proximity to a bleeding injury of moderate to high flow to achieve hemostasis can result in excessive material application with consequences such as the adverse event of retained endoscope [21].

Benign Prostatic hyperplasia (BPH) and prostate cancer are two of the most common urologic diseases that are treated with surgical intervention in aging men. An estimated 50% of men have histologic evidence of BPH by age 50 years and 75% are thought to display such evidence by age 80 years. In 40-50% of these patients, BPH becomes clinically significant. Although the incidence of uncontrolled bleeding from surgical intervention involving prostate and urethra is relatively low, it remains a significant risk that must be addressed by in hospital with a length of stay over at least two to three nights. According to statistical analysis of U.S. Department of Health and Human Service from 2005 to 2010, there was an average of 150,000 discharges from either open or transurethral procedure prostatectomy in the U.S., with direct surgery cost surpassing an average $4.5 billion annually. In these patients, the average length of hospital stay with open or transurethral prostatectomy was 3.1 and 2.4 days respectively. In the patients who had blood transfusion due to significant blood loss (4-5%) in the surgery, the average length of stay was prolonged to five or six days that cost an average $15,700 more in each case compared to the average cost ($29,300) of prostatectomy patients in 2010. The costs of the prostatectomy procedure are high because of operating-room time, surgeon time, and hospital length of stay.

TURP is considered the benchmark therapy for BPH. Partial removal (resection) of the prostate is accomplished in TURP by minimally invasive surgery through the urethra using a cystoscope (endoscope for the bladder via the urethra) and electrocautery. The thin loop electrocautery used in TURP results in less tissue necrosis than other less common minimally invasive prostatectomy procedures, however there is more intraoperative bleeding with TURP. Appropriate prostate resection and control of bleeding in TURP, like other forms of prostatectomy, are its essential challenges. The volume of the intraoperative bleeding in prostatectomy depends on the size of the prostate, the length of time to resect the prostate, and the surgeon's skill. Significant bleeding or hemorrhage after prostatectomy often causes undesirable clot retention (and resulting urinary retention) in the bladder and urethra that may prolong time in hospital, and even necessitate re-operation. In general, arterial bleeding is easily identified and controlled by electrocoagulation, but the venous bleeding common in TURP is more difficult to control. This is because highly vascular organs and glands such as the prostrate provide for large surface area continuous oozing bleeding from the resected injury site and not just pin-point arterial vessels which are easily controlled by spot cautery. The resected surface of the prostate will continue to demonstrate problematic oozing bleeding for up to seventy two hours in a significant number of patients. Attempts to control venous bleeding by electrocautery and irrigation may result in undesirable outcomes such as TURP syndrome. In standard of care control, venous bleeding is controlled by filling the bladder with irrigating fluid and application of an inflated transurethral balloon catheter to compress the bleeding prostatic cavity. TURP associated post-operative morbidity rate has been reported as high as 18% with an operative mortality rate of 0.3%. In older patients, the risk of blood loss related morbidity and mortality increases significantly in association with coagulation disorders and cardiovascular abnormalities. Uncontrolled bleeding during TURP is still one of the major complications of prostate resection and this often leads to converting to less desirable open surgery. Although there is significant progress in the management of BPH, the incidence of uncontrolled strong bleeding remains around 6% and blood transfusion rate to address this bleeding is 4% to 5%.

In the typical TURP, the length of hospital stay is two to four days and the patient has an inflated urinary balloon catheter in place until bleeding stops and urine becomes clear. Any significant reduction of post-op bleeding following TURP will shorten time of catheterization and hospital bed requirements. It will also decrease the incidence of urinary tract infection, catheter-related patient discomfort and related complications. Significant hematuria (blood in urine), resulting from either transurethral or open surgery, which causes hemodynamic instability and clot retention, requires immediate medical attention and medical care for hemostasis, clot clearance, blood transfusion, and coagulation evaluation. Treatment of significant hematuria through a transurethral approach is troublesome due to limited operative visual and spatial restriction. Most often the patient has to return to the operating room to perform an open bladder surgery to achieve hemostasis and remove cystic clots. A complicating factor of prostatectomy is that TURP patients are commonly anti-coagulated due to the presence of other chronic conditions such as cardiovascular disease. Although it is preferable to have these patients taken off their anti-coagulation medication such as Coumadin and Plavix before TURP surgery because of risk of bleeding, it would be preferable to be able to perform the procedure while the patient remains on their medication to reduce the possibility of stroke or myocardial infarction during the procedure. A reliable and sustainable hemostatic technique, preferably effective in the case of anti-coagulated individuals, is urgently required for the transurethral application to control significant bleeding following prostate resection.

Chitosan materials have been used in the art to address hemorrhage in varying applications and with varying degrees of success, but advances and improvements are badly needed to unlock the potential of chitosan materials, such as catechol modified chitosan, to provide alternative and better chitosan-based solutions for safe, reliable and effective delivery of bioactive chitosan material within the body and, particularly, within challenging in vivo biological environments.

### BRIEF SUMMARY

The present disclosure relates to easy to deliver, flowable, fluidized (the carrier fluid is a liquid), dispersed-particle, tissue adherent chitosan materials and their compositions that resist dissolution, provide persistent adherence to tissue and are highly effective in promoting rapid hemostasis. These chitosan materials comprise catechol modified chitosan and are generally referred herein to as “flowable dressings” and such term is used to refer to the fluidized, dissolution-resistant, tissue adherent particles formed from the chitosan materials of the present invention. The flowable dressings described herein are readily delivered locally by, for example, a catheter, and are suitable for use in minimally invasive procedures.

It is to be understood that flowable chitosan materials described herein have numerous beneficial properties arising from the chitosan material itself, and that these chitosan materials may take numerous potential reduced particle forms. Such reduced particle forms may be created by processes including but not limited to granulating (larger solid forms into course granules>1 mm in diameter), milling (converting granules to powders<0.5 mm in diameter), grinding (reduction in powders to <0.2 mm in diameter), sieving (to select for particle size), spray drying (direct forming of small solid particles from liquid without milling or grinding), and chopping (of micron and sub-micron diameter fiber to produce chopped, low aspect ratio fiber of length:diameter<200:1). It is to be understood that the reduced particle forms of the invention remain as solid materials in both their dry and their wet flowable configurations. In the presence of liquid, the particles may be present as solid and semi-solid material. The solid particle forms of the disclosure may swell in volume (<50%) but remain solid in the presence of liquid. These solid materials remain as discrete individual particles and they retain their essential solid property by resisting both dissolution and deformation. The semi-solid particle forms of the disclosure may swell in volume (≥50%). These semi-solid materials remain as discrete individual particles and they retain their essential semi-solid property by resisting dissolution while demonstrating swelling deformation in shape. The dry semi-solid particle may have an original appearance of a course, sharp edged grain which when exposed to liquid becomes enlarged and swollen to a more rounded, possibly spherical shape. A preferred form of milling and grinding is at or below −40° C. such as in the presence of dry ice. A more preferred form of milling and grinding is at cryogenic temperature at or below-180° C. such as in the presence of liquid argon or liquid nitrogen.

In one embodiment, the chitosan particles, including the catechol modified chitosan particles with their chitosan Schiff base crosslinking, in the presence of the diluent carrier liquid and in the presence of biologic fluids such as blood, gastrointestinal fluid, and urine, remain mostly insoluble as solid or semi-solid discrete particles to provide a high specific surface area of suspended solid in the diluent carrier liquid. And even under instances of some level of clumping and aggregation of the chitosan particles, the essential discrete nature of the particles is retained.

Beneficial properties characteristic of, and arising from, the chitosan materials described herein include, but are not limited to, the ability to tune the material by controlling the degree of substitution and level of oxidation. By tuning the material by controlling the degree of substitution and level of oxidation, the chitosan materials can resist fast degradation and dissolution in difficult biological environments at temperatures near 37° C. such as the GI tract, the urethra, the lower GI tract, internal body cavities such as the abdominal and thoracic cavities, but still be sufficiently soluble to go away in less than about seven (7) days. It is to be understood that the dissolution resistant materials described herein confer a degree of degradation resistance. This dissolution resistance protects the materials against degradation when exposed to harsh wet environments such as acid and enzyme rich environment of the upper gastrointestinal tract and the enzyme and urea rich environment of the bladder. Additional beneficial properties of the chitosan materials are that it is also biocompatible, hemostatic and tissue adherent. Also, the hemostatic mode of action of the chitosan material avoids risk of emboli and thrombi. The chitosan material also promotes normal healing of injuries with reduced risk of scar formation.

The flowable dressing chitosan material described herein can be delivered accurately as a final fluidized dispersion to a remote injury site by, for example, catheter delivery to adhere to the tissue of the remote site independent of gravity to quickly effect bleeding control, close the injury site and stay in place resisting dissolution for up to and more than 6 hours. The flowable dressing chitosan material of the disclosure adheres on contact, and for greater than 6 hours, to mucosal tissue and tissue injury sites. The flowable chitosan material of the disclosure may be applied upside down under normal gravity to adhere to an injury site with endoscopic application without loss of coverage or without flowing away from its site of application. After 6 or more hours of application to an injury site, the top portion of the applied flowable dressing chitosan material may be eroded or bio-dissolved but the flowable dressing material closest to the injury remains adhered as a thin, uniform layer covering the injury site for at least 12 hours to protect the injury site and reduce opportunity for rebleeding. The flowable dressing of the disclosure may be used with minimally invasive techniques for remote dressing delivery to quickly deploy dressing to achieve hemostasis, to fill and to close resections, biopsy sites, narrow recesses, defects and openings around hemostatic clips, sutures, clamps, staples, wires and pins.

The flowable dressing material and its compositions described herein are provided as non-limiting examples of final forms of the present invention. The flowable dressings described and exemplified herein provide examples of the flowable catechol modified chitosan material compositions and their attributes. Further, the flowable chitosan compositions include, but are not limited to fluidized solid and semi-solid particles. The solid particle flowable material of the invention is dispersed in a diluent carrier liquid to form a fluidized dispersion of solid particles that may become swollen with the diluent liquid but remain resistant to dissolution of the particles. It is understood that the dispersion, sometimes termed “suspension”, of the particles in the fluid results in dispersed, sometimes termed “suspended”, particles. In such dispersed particle liquid systems, the liquid phase is described as the continuous phase while the dispersed particle phase is termed the discrete phase. It is understood that the fluidized systems of the disclosure are the combination of continuous liquid carrier at greater than 75% w/w and discrete particles at not more than 25% w/w. The liquid carrier in a dispersion is a non-solvent, alternately termed “diluent”, for the discrete particle phase.

In a preferred embodiment, the flowable material of the disclosure, immediately on mixing discrete particle and liquid components, has viscosity greater than the viscosity of the carrier liquid which in the case of water at 20° C. is 1 mPa·s. In a preferred embodiment, the discrete particle and the liquid components may be mixed together in the presence of atmospheric gases without any foam formation and with all the gases being substantially absorbed by the liquid and discrete particle dispersion. Preferably the uniform mixing of the liquid and discrete particle components is achieved in no more than 180 seconds, more preferably the mixing of the liquid and discrete particle components is achieved in no more than 60 seconds, and most preferably the mixing of the liquid and discrete particle components is achieved in no more than 30 seconds. Also, the mixed flowable material viscosity is not more than the viscosity of a fluid which can be delivered through a 1.5 meter length of tube or channel with internal diameter of 1.5 mm connected to a 5 ml syringe with 12 mm internal diameter barrel and delivered with maximum barrel load of 50 kgf at more than 5 ml/min. Once delivered at the injury site at 37° C., the flowable dressing adheres to the wound tissue and has resistance to flow so that it remains conformed over the injury without flowing under gravity away from its site of application. In one embodiment, the flowable composition of the dressing may be mixed up to 2 hours before its intended use without losing any of its desired flowable and hemostatic properties. In one embodiment, the flowable composition of the dressing may be mixed up to 1 hour before its intended use without losing any of its desired flowable and hemostatic properties. In one embodiment, the flowable composition of the dressing may be mixed up to 45 minutes before its intended use without losing any of its desired flowable and hemostatic properties. In one embodiment, the flowable composition of the dressing may be mixed up to 30 minutes before its intended use without losing any of its desired flowable and hemostatic properties. In one embodiment, the flowable composition of the dressing may be mixed up to 15 minutes before its intended use without losing any of its desired flowable and hemostatic properties. In one embodiment, the flowable composition of the dressing may be mixed up to 10 minutes before its intended use without losing any of its desired flowable and hemostatic properties. In one embodiment, the flowable composition of the dressing may be mixed up to 5 minutes before its intended use without losing any of its desired flowable and hemostatic properties.

In a preferred embodiment of the flowable dressing, the viscosity of the diluent carrier liquid in which the dissolution resistant particles are suspended is equal to or less than the viscosity of blood at 37° C. which is between 3.5 and 5.5 mPa·s The lower viscosity of the carrier liquid (relative to the viscosity of blood at 37° C.) provides for ability of blood to dilute and displace the carrier liquid as the continuous phase supporting the solid particles of the flowable dressing, and therefore provides for the ability of blood to interact with a high specific surface area (e.g., >100 cm²/g) of the flowable dressing.

In one embodiment, the flowable composition comprises a particulate chitosan material and a diluent carrier liquid. In one embodiment, the flowable composition comprises a particulate chitosan material comprises an amount of greater than or equal to about 5% of the total weight of the flowable composition. In one embodiment, the flowable composition comprises a particulate chitosan material provided as a solid or semi-solid and, optionally, wherein the semi-solid particulate chitosan material is swollen. In one embodiment, the flowable composition comprises a particulate chitosan material is provided as a powder, a granule, particle, fiber, or any combination thereof. In one embodiment, the flowable composition comprises a particulate chitosan material comprises regular or irregular shaped particles with radius of gyration in the range of about 10 to 350 micrometers. In one embodiment, the flowable composition comprises a particulate chitosan material comprising one or both of catechol modified chitosan and chitosan gelatin crosslinked. In one embodiment, the flowable composition comprises a particulate chitosan material comprising one or more of a densified chitosan material, a freeze-phase-separated and dried chitosan material, a densified freeze-phase-separated and dried chitosan material, a spray-dried chitosan material, a dried cast film chitosan material, a freeze-phase-separated chitosan material that is dried by freeze substitution, a sublimated freeze-phase-separated chitosan material, a dried freeze thawed chitosan material, and a dried asymmetrical centrifugally mixed material. In one embodiment, the flowable composition comprises a diluent carrier liquid that may be selected from a group comprising one or more of water, standard 0.9% aqueous saline solution, and autologous plasma. In one embodiment, the flowable composition comprises a diluent carrier liquid that may comprise at least one of about 85% of the total weight of the flowable composition, about 90% of the total weight of the flowable composition, or about 95% of the total weight of the flowable composition. In one embodiment, the flowable composition comprises a diluent carrier liquid that may comprise a thermoresponsive fluid capable of delivery through a 23-gauge needle or a 24-gauge needle at about 18-25° C. and capable of gelation at about 37° C. In one embodiment, the flowable composition is hemostatic. In one embodiment, the flowable composition comprises a diluent carrier liquid that may comprise is resistant to dissolution. In one embodiment, the flowable composition comprises particulate chitosan material that does not substantially dissolve and remains solid or semi-solid. In one embodiment, the flowable composition is capable of resisting dissolution in at least one of urine, water, saline solution, blood, or gastrointestinal (GI) fluid at about 37° C. for at least about 6 hours. In one embodiment, the flowable composition comprises a particulate chitosan material that is further characterized by presentation of a specific surface area greater than about 100 cm² per gram of flowable dressing. In one embodiment, the flowable composition comprises at least a first outer layer and a second tissue adherent layer, and wherein the first outer layer resists dissolution for at least about 6 hours and the second tissue adherent layer resists dissolution for at least about 12 hours. In one embodiment, the flowable composition is tissue adherent. In one embodiment, the flowable composition is tissue adherent and adheres on contact for a period of time that is greater than about 6 hours to at least one of mucosal tissue and a tissue injury site. In one embodiment, the flowable composition may be endoscopically applied upside down under normal gravity and adhere to tissue. In one embodiment, the flowable composition is biocompatible. In one embodiment, the flowable composition is capable of delivery to a tissue site via a channel having a diameter of at least one of less than about 7 mm, less than about 4.5 mm, less than about 4.0 mm, less than about 3.2 mm, less than about 2.8 mm, and about 0.5 mm.

In one embodiment, the flowable composition comprising a particulate chitosan material and a diluent carrier liquid is an endoluminal hemostatic dressing, and methods of making and administering said flowable compositions.

In one embodiment, the flowable composition comprising a particulate chitosan material and a diluent carrier liquid is a gastrointestinal hemostatic dressing and methods of making and administering said flowable compositions. In one such embodiment, the disclosure provides for a method of administering the flowable composition comprising sealing of the tissue site by the gastrointestinal hemostatic dressing for at least six hours in an acid environment of about pH 3. In one such embodiment, the disclosure provides for a method of administering the flowable composition comprising providing for the dissolution of the gastrointestinal hemostatic dressing from the tissue site over a period of time less than or equal to about seven days.

Another embodiment of the present disclosure relates to a method of making a flowable composition described herein. In one embodiment, such a method comprises preparing a chitosan material for use in the flowable composition. In one embodiment, such a method comprises preparing one or both of catechol modified chitosan and chitosan gelatin crosslinked chitosan material. In one embodiment, such a method comprises preparing a chitosan material that is one or more of a densified chitosan material, a freeze-phase-separated and dried chitosan material, a densified freeze-phase-separated and dried chitosan material, a spray-dried chitosan material, a dried cast film chitosan material, a freeze-phase-separated chitosan material that is dried by freeze substitution, a sublimated freeze-phase-separated chitosan material, a dried freeze thawed chitosan material, and a dried asymmetrical centrifugally mixed material. In one embodiment, such a method comprises preparing a chitosan material and grinding the chitosan material to form the particulate chitosan material.

One embodiment of the present disclosure provides a method of delivering a flowable composition to a tissue site to a subject in need thereof comprising combining the particulate chitosan material and a diluent carrier liquid prior to delivery to the subject. In one such embodiment, the method further comprises providing the particulate chitosan material and the diluent carrier liquid as separate components for combination and, optionally, separately sterilizing the separate components. In one such embodiment, the method comprises delivering an amount of the flowable composition sufficient to achieve hemostasis at a bleeding tissue site of the subject. In one such embodiment, the method further comprises delivering the flowable composition to the tissue site via a channel having a diameter of at least one of less than about 7 mm, less than about 4.5 mm, less than about 4.0 mm, less than about 3.2 mm, less than about 2.8 mm, and about 0.5 mm. In one such embodiment, the method further comprises delivering the flowable composition to the tissue site in one or more layers and, optionally, wherein the flowable composition comprises at least a first outer layer and a second tissue adherent layer, and wherein the first outer layer resists dissolution for at least about 6 hours and the second tissue adherent layer resists dissolution for at least about 12 hours. In one such embodiment, the method further comprises adhering the flowable composition to the tissue site and, optionally, wherein the flowable composition adheres to the tissue site upon contact and for a period of time that is greater than about 6 hours, and wherein the tissue site comprises at least one of mucosal tissue and a tissue injury. In one such embodiment, the flowable composition may be endoscopically applied upside down under normal gravity and adhere to a tissue site.

In one embodiment, the present disclosure provides a method of delivering the flowable composition comprising a particulate chitosan material and a diluent carrier liquid to a tissue site to a subject in need thereof comprising: combining the particulate chitosan material and a diluent carrier liquid prior to delivery to the subject; applying the flowable composition; and adhering the flowable composition to the tissue site upon contact. In one such embodiment, the diluent carrier liquid has a viscosity that is less than or equal to 3.5 and 5.5 mPa·s. In one such embodiment, the method further comprises applying the flowable composition using minimally invasive techniques and, optionally, wherein the minimally invasive techniques provide for remote flowable composition delivery. In one such embodiment, the method further comprises applying the flowable composition to do one or both of filling and closing of resections, biopsy sites, narrow recesses, and defects and openings around hemostatic clips, sutures, clamps, staples, wires and pins.

The present disclosure generally relates to flowable chitosan catechol modified dressing compositions that, given their characteristics, can be applied in different physiological settings to stop bleeding and provide prolonged wound protection. Embodiments include, among others, chitosan gastrointestinal hemostatic flowable dressings (CGHFD) and chitosan endoluminal hemostatic flowable dressings (CEHFD).

A flowable dressing material described herein has the following combination of one or more, or all, properties: it (1) is able to be delivered rapidly and accurately from a syringe or other delivery instrument through a catheter or other tubing delivery vehicle to remote locations; (2) can be applied in the presence of actively flowing blood and other biological fluids at about 37° C. without significant dimensional changes in length, width and height to effect rapid hemostatic control of bleeding; (3) is able to be delivered in the presence of biological fluids and blood; (4) is able to be delivered to a surgical site manually or by a minimally invasive delivery device; (5) is able to be in an inverted orientation (upside down) and adhere to its application site and maintain its original shape without material loss by flowing or dripping; (6) is able to be applied as a non-occlusive, flowable material to maintain a dispersed solid and semi-solid specific surface area greater than 100 cm²/g to promote blood cellular interactions as well as adsorb hydrophilic and hydrophobic biological fluids that can interfere with adhesion; (7) maintains an interconnected porosity≥5 microns radius between individual flowable dressing chitosan particles; (8) adheres to mucosa, resected mucosa and resected tissue on application; (9) is able to uniformly adhere to tissue and quickly promote local blood clot formation without risk of remote emboli or thrombi; (10) is able to be released from a delivery catheter to allow withdrawal of the catheter from the surgical site; (11) is able to resist dissolution and provide prolonged (persistent) adherence to tissue for up to 48 hours on exposure to enzymatic wet biological environments containing biological fluids in the range of about pH 3.0 to about pH 8.0 at about 37° C.; (12) enables (with or without delivery device in place) ostomy connecting channels such as those in the gastrointestinal tract and the urethra to remain patent with material or residues of material present and unobstructed passage of excretions; (13) protects the injury site and provides for promotion of healing with reduced risk of scar formation; (14) provides a controlled, slow degradation and/or dissolution from the attachment site to allow for removal without surgical assistance in less than seven days in ostomy uses; (15) the flowable material may include one or more of a dispersed milled powder, a dispersed ground powder, a dispersed spray-dried powder, a dispersed chopped fiber, and may include combinations of these different dispersions; (16) the tissue adherent flowable material of the invention may include, but is not limited to, one or more uses as an adherent dressing, an adherent hemostatic dressing, an adherent patch for localized controlled release of an active agent, a material for promotion of clotting, an tissue adherent matrix material for tissue regeneration, and a tissue adherent matrix material for sealing of vascular malformations; (17) the tissue adherent flowable material of the invention may include, but is not limited to, use in combination with a suture, a staple, a hemostatic clip, a hemostatic clamp, an orthopedic bone fixture, or an occlusive suture-less patch; (18) the tissue adherent flowable material of the invention may be used as part of a combination therapy for definitive control of difficult bleeding where the flowable dressing may be applied initially to stanch hemorrhage providing sufficient injury site visualization for application of other hemostatic therapies including but not limited to one of injection of epinephrine, clamping, cautery, and suture. In one embodiment, porosity is uninterrupted in the flowable dressing with interconnected pore size range of 10-100 microns with substantially most of the pores near 10-50 microns. The uninterrupted pore structure is indicated in the flowable dressings by their ability to absorb biological fluid such as blood. Difficult to control bleeding occurs commonly in patients on anticoagulation medication and those with disorders of bleeding. It can occur during a surgical procedure from injuries related to disease, tissue failure, and surgical error. It is a serious problem in minimally invasive surgical procedures when standard bleeding control hemostatic techniques fail, blood product transfusion becomes necessary, and the only option to control the bleeding becomes conversion to high risk open surgery.

Although existing tools in the United States readily control a significant portion of UGIB, there remains unmet need for the safe flowable dressing materials of the disclosed herein that provides rapid control of bleeding. Broad application of the subject flowable dressing materials described herein could enable significant reduction in morbidity and mortality in gastrointestinal bleeding treatment with concomitant reduction in associated health care expenditure.

The subject flowable dressing materials disclosed herein are amenable to use in all gastrointestinal bleeding applications and may be delivered as a chitosan gastrointestinal hemostatic flowable dressing (CGHFD) by, for example, a catheter through a standard endoscopic working channel (≤3.8 mm diameter). The subject material of the invention will provide an opportunity to address or mitigate deficiencies with current modalities, such as clipping, thermal coagulation and injection, which necessitate pinpoint accuracy which is challenging under impaired visibility of brisk bleeding conditions. Thermal coagulation is also problematic because of its propensity to induce adverse scar tissue formation.

The subject flowable dressing material described herein is amenable to use in all transurethral resection of the prostate bleeding applications and may be delivered as a chitosan endoluminal hemostatic flowable dressing (CEHFD) by, for example, a transurethral balloon catheter channel. The subject material will provide opportunity to address or mitigate deficiencies with current modalities, such as application of traction to the bleeding site through a Foley catheter or use of biologic flowable systems such as FLOSEAL which do not remain in place in the presence of urine.

The subject flowable dressing material described herein is amenable to use in other procedures outside gastrointestinal and transurethral bleeding control such as control of bleeding in surgical procedure including but not limited to maxillofacial surgery, otolaryngology (ENT)/head and neck surgery, bladder surgery, oral surgery and may be delivered as a chitosan hemostatic flowable dressing (CHFD) by, for example, a catheter channel. The subject material of the invention will provide opportunity to address or mitigate deficiencies with current modalities, such as application of traction to the bleeding site or use of biologic flowable systems which do not remain in place in wet or inverted environments.

The present invention comprises flowable dressing materials and compositions, methods of using the compositions, and methods of making the compositions.

In a preferred embodiment, the flowable dressing material comprises a tissue adherent fluidized particle composition wherein the fluidized tissue adherent particles comprise dissolution-resistant crosslinked chitosan particles.

In a preferred embodiment, the flowable dressing material comprises a tissue adherent fluidized particle composition wherein the fluidized tissue adherent particles comprise dissolution-resistant crosslinked chitosan particles that provide for a high specific surface area interaction with an injury site with adherence of the particles to the site of greater than 6 hours.

In a preferred embodiment, the flowable dressing material comprises a tissue adherent fluidized particle composition wherein the fluidized tissue adherent particles comprise dissolution-resistant, crosslinked chitosan particles that provide for a high specific surface area interaction with an injury site with attachment of the particles to the site of greater than 6 hours that results in rapid and prolonged hemostasis at the injury site with protection from rebleeding and reinjury.

In a preferred embodiment, the flowable dressing material composition is of sufficient volume of material, preferably 3 ml, more preferably 5 ml most preferably 7 ml, so that in one delivery there is enough material to cover, to a depth of 2 to 5 mm, a typical peptic ulcer bed of 20 mm diameter in an upper gastrointestinal injury. In a preferred embodiment, the flowable dressing material composition is of sufficient volume of material, preferably 5 ml, more preferably 7 ml most preferably 10 ml, so that in one delivery there is enough material to cover to a depth of 7 to 15 mm with balloon tamponade a resected prostate fossa injury 30-50 mm long, 15 mm deep and 10 mm wide. It is understood that the flowable dressing of the disclosure may be applied in multiple applications to the same injury should the injury be larger than the typical injury size or the bleeding sight prove difficult to control with a single application.

In a preferred embodiment, the flowable dressing material composition may be delivered in a continuous stream under close to constant pressure from a tubing distal end such as from a catheter to fill a cavity or alternatively provide a uniform coating over a bleeding wound. In an alternate embodiment, the flowable dressing material may be delivered at regular and irregular discrete volumes in a discontinuous fashion from the delivery tubing distal end under bursts of pressure to provide for elevated momentum of the flowable dressing application onto target tissue surface. The resultant discrete volume sputtering application may be used to build a 3-dimensional dressing structure by repeated, discrete, flowable, print-like delivery to the deposited dressing body.

In a preferred embodiment, the flowable dressing material comprises a catechol modified chitosan, wherein the catechol modified chitosan material is preferably at least 25% w/w, more preferably at least 50% w/w and most preferably at least 75% w/w of the dispersed dry solid in the flowable composition. The catechol modified chitosan flowable dressing can adhere to an applied injury site immediately on application. The flowable dressing can form a quaternary ammonium cation at the chitosan glucosamine C-2 amine at a tissue injury site. The flowable dressing may comprise catechol oxidized to o-quinone and cross-linked in the chitosan. In one embodiment, the flowable chitosan dressing may have a brown coloration, including a dark brown to black coloration. In one embodiment, the fluidized dressing particles may comprise catechol that has a low level of oxidation, and wherein the flowable chitosan dressing has appearance of brown to pink coloration.

The particles of the flowable dressing may include but not be limited to powders and fibers.

In a preferred embodiment, the dry particles of the flowable dressing may be formed from one or more of spray-dried, dried cast film, sublimated freeze separated, solvent substituted freeze separated, dried freeze thawed, dried asymmetrical centrifugally mixed, extruded and continuous fiber compositions containing catechol modified chitosan. A solid powder may be formed by grinding and milling processes from solid films, sheets, rods, granules and chips of compositions containing catechol modified chitosan. A preferred grinding and milling process is cryo-grinding at less than −40° C.

In a preferred embodiment the original dry particles, prior to forming the tissue adherent fluidized particles of the flowable dressing, may be regular or irregular shaped particles with radius of gyration in the range of 10 to 350 micrometers.

In a more preferred embodiment the original dry particles, prior to forming the tissue adherent fluidized particles of the flowable dressing, may be irregular or regular shaped particles with radius of gyration in the range of 10 to 200 micrometers.

In a most preferred embodiment the original dry particles, prior to forming the tissue adherent fluidized particles of the flowable dressing, may be irregular or regular shaped particles with radius of gyration in the range of 10 to 100 micrometers.

In a preferred embodiment, the dry, solid, particle fraction of the flowable dressing composition is sterilized separately of the carrier liquid diluent fraction. Sterile dry solid and liquid fractions are combined aseptically immediately before use to form the flowable fluidized dressing of the invention.

In a preferred embodiment, the sterile, dry solid particle fraction of the flowable dressing is present inside a first closed syringe and the sterile, liquid carrier fraction is present in a second syringe with both first and second syringes being able to be connected aseptically together to combine and mix the liquid and solid particle components into the final flowable form immediately before delivery and use.

Sterilization of the solid fraction may include but not be limited to gamma-irradiation, electron beam irradiation, x-ray irradiation and ethylene oxide gas exposure.

The carrier liquid diluent fraction of the flowable dressing may be sterilized separately from the solid fraction using sterilization methods including, but limited to, sterile filtration, gamma irradiation, electron beam irradiation, and x-ray irradiation. Alternatively, the fluid fraction may be prepared aseptically from a sterile source.

The carrier liquid diluent of the flowable dressing may include, but is not limited to, one or more of water, standard 0.9% aqueous saline solution, and autologous plasma.

The carrier fluid may be a thermoresponsive fluid that provides for fluidized particle delivery through a catheter and narrow gauge needle such as, for example, 24 to 23 gauge needles at room temperature (18-25° C.) and provide for gelation (near solidification) at 37° C. in the body. Such delivery is especially desirable in esophageal submucosal dissection where you need to lift the submucosa with a readily flowable lifting agent but desire the fluid remain in place with a flowable hemostatic agent after lifting.

In a preferred embodiment, the carrier liquid fraction of the flowable composition is preferably no more than 95% w/w, more preferably no more than 90% w/w and most preferably no more than 85% w/w of the total weight of the flowable dressing (total weight=solid weight fraction+liquid weight fraction).

The dressing adherence strength may be greater than or equal to about 1 kPa. The dressing resists dissolution in urine, water, saline solution, blood, or GI fluid at about 37° C. for at least about 6 hours. The angle of delivery and rate (milliliters/min) of delivery combined with delivery catheter internal diameter and tip shape will determine shape and volume of delivered flowable dressing. Tip shapes used to accomplish release of the flowable dressing from the delivery catheter (or tube, channel, etc.) may vary, and include and are not limited to a tip with a narrowed end, a narrow splayed end, actuated release, a print head sputtering-type system that may be used with controlled velocity to deliver different size deposits at different momenta with big deposits over large bleeds, smaller deposits to cover (or print) over large areas, etc.

The flowable dressing may be applied as a tissue adhered, thin uniform coating that can be applied to a broad tissue area in one layer or layer upon layer or in one or more deposits to a target site or would coverage area. The flowable dressing may be applied as a tissue adhered, dressing bead that can be applied to fill and approximate injury openings. In one embodiment, the flowable dressing may be ‘painted’ in a layer of about 1-2 mm thickness, or layered to about 5-7 mm thickness, or used to fill cavities of, for example, 10-35 mm depth.

The flowable dressing may be applied in combination with other materials, including application into, around and through previously applied patch type dressings, clips and clamps to immediately stanch all oozing bleeding. The flowable dressing may be applied broadly over bleeding surfaces as a preferred non-cauterizing hemostatic treatment to provide for desired wound healing with minimal to no scar production compared to the highly scarring and poor wound healing effects of standard hemostatic thermal cautery treatments. The dressing readily releases from its delivery catheter by pinching the flowable dressing bead at the distal end delivery tube after adherence to a target tissue site to separate the dressing from the delivery catheter since the flowable dressing tissue adherence is greater than the cohesive strength of the flowable bead at the end of the delivery tube. Alternate means of separation of the dressing from its delivery device in application may include pinching the tip of the catheter against the wound or other tissue, flicking the end of the catheter, cutting or gating the flowable dressing stream and/or applying the dressing in discrete, discontinuous volume bursts.

The dressing is able to resist dissolution for at least six hours after adhering to an injury site in presence of corrosive enzymes and acid environment of about pH 3. The dressing is able to seal and protect a target tissue site for at least 6 hours. The dressing is able to achieve a controlled, slow dissolution from the attachment site over a period of time not exceeding seven (7) days. The dressing is not readily soluble in water, saline solution, blood, or GI fluid at about 37° C. for at least 6 hours following application. The dressing is not readily soluble in water, saline solution, blood, or GI fluid at about 37° C. for at least 12 hours following application. The dressing does not adhere or build-up residue to the delivery device due to the non-stick material surface of the delivery device and the shear of the material against itself during delivery.

In one embodiment, the flowable dressing is like thick ketchup and as it flows along the delivery tube it just pushes everything in front along before it, and it is non-setting (i.e., there no chemical reactions happening) so its stays wet, is only sticky enough to adhere to tissue and itself once delivered, and it reacts with blood to produce a clot, so when added to a bloody environment the dressing exhibits and is characterized by enhanced cohesion and adhesion to the wound.

In a preferred embodiment, the flowable dressing comprises an optical contrast material that provides enhanced endoscopic visualization of a deployed flowable dressing for improved wound placement and post placement observation of the wound and dressing. The material can be mixed readily with the composition of the flowable dressing and delivered with the flowable dressing to address bleeding and provide for enhanced ability to visualize the dressing edges, the body of the dressing and successful hemostasis. The enhanced visualization material remains bound and uniformly present within the flowable dressing composition without any significant leakage of the material into the biological environment while the flowable dressing is providing hemostasis. Enhanced visualization materials may include but not be limited to fluorescent agents, nanoparticles containing fluorescent agents, chitosan covalently modified with a fluorescent agent, quantum dots, gold nanoparticles, organically modified dye-doped silica, upconverting phosphors and lanthanide-based contrast agents.

In a preferred embodiment, the flowable dressing hemostasis may be augmented by endoscopic placement of a supportive solid mesh or dressing over the flowable dressing once the flowable dressing has been deployed over a bleeding site. In the case of strong arterial bleeding which may prove too high a pressure for the cohesive strength of the flowable dressing to immediately control bleeding due to tunneling or other penetration of the arterial bleeding pressure through the flowable dressing, then application of light tamponade pressure for a short period by balloon or similar endoscopically applied basket device through an intermediary dressing or mesh material over the flowable dressing. The application of the support dressing or mesh with light pressure closes any tunneling or other penetration within the flowable dressing and provides for prolonged hemostasis under difficult bleeding conditions.

The dressing does not increase or decrease in size by more than about 25% in length and width, or more than about 50% in thickness in the presence of water, saline solution, blood, or GI fluid at about 37° C. at the wound site. The separate flowable dressing components are capable of being stored in their packaging at or below room temperature (25° C.) for 2 years or more without affecting dressing characteristics.

The dissolution resistant, tissue adherent, flowable hemostatic dressing of the invention may be used by itself or as an adjunct hemostatic dressing to control all bleeding in patients with normal blood clotting, and also in patients on anti-coagulation therapy, anti-platelet therapy and with disorders of bleeding.

The dissolution resistant, tissue adherent, flowable hemostatic dressing of the invention may be applied to an injury site to remain in place, control bleeding and promote rapid tissue regeneration at the site of an injury with reduced risk of scar formation.

UGIB bleed rates, or blood flow rates, in ml/min suitable for treatment by the flowable hemostatic dressing described herein may range from about 1 ml/min to about 100 ml/min. In preferred embodiments, the bleeding rates addressed by the devices range from about 1 ml/min to about 40 ml/min. A Forrest 1a UGIB is about 25 ml/min. For subjects suffering a bleed rate of much greater than a Forrest 1a, survival is unlikely unless they are already in an operating theater. UGIB bleed rate of between about 20 ml/min and 25 ml/min is considered “brisk” bleeding. Oozing bleeding is generally greater than about 1 ml/min as it is noted that low bleeding rates such as 1 ml/min typically clot and stop of their own accord unless the subject is on anticoagulation therapy or has a disorder of the clotting cascade due to reasons other than taking anticoagulation medication. For such a subject with irreversible anticoagulation medication or with a bleeding disorder, 1 ml/min oozing bleeding remains concerning and needs to be addressed such as by the flowable dressing of the disclosure. In some embodiments, the flowable dressings described herein are used to address UGIB bleeding rates of between about 1 ml/min and about 25 ml/min, or about 1 ml/min and about 20 ml/min, or about 1 ml/min and about 15 ml/min, or about 1 ml/min and about 10 ml/min, or about 1 ml/min and about 5 ml/min. In some embodiments, the dressing can be used for treatment of a disease, condition, disorder, trauma, or injury. For example, the use of the dressing in the treatment of a disease, condition, disorder, trauma, or injury, comprising directly adhering the dressing at an injury site. The dressing for use in treatment of a disease, condition, disorder, trauma, or injury, may remove (through high specific area adsorption) anti-adhesive hydrophilic and hydrophobic biological fluids that would normally interfere with adherence. The dressing for use in treatment of a disease, condition, disorder, trauma, or injury, may comprise leaving the dressing in place at a target tissue site and the dressing may remain at the target tissue site for at least 12 hours. The dressing for use in treatment of a disease, condition, disorder, trauma, or injury, may be capable of slow dissolution at the target tissue site and dissolves completely without human intervention in less than or equal to seven days.

In some embodiments, the invention disclosed herein comprises methods of producing the flowable chitosan dressing. In one embodiment, the method comprises: performing synthesis with chitosan and catechol to produce the modified catechol chitosan in an aqueous reaction solution; maintaining a pH of the reaction solution at or below pH 5.5; increasing the pH of the reaction solution, and controlling oxygen exposure to the reaction solution, to provide catechol oxidation and crosslinking; and drying the reaction solution. Another embodiment of a method of producing a freeze-dried modified catechol chitosan sheet comprises: freeze-drying an aqueous solution comprising the modified catechol chitosan material; obtaining a freeze dried chitosan solid with inter-connected porous structure from each of the above steps. Another embodiment of a method of producing a freeze-dried modified catechol chitosan sheet includes compressing the freeze dried modified catechol chitosan material to a density greater than 0.5 g/cm³. In certain embodiments, the compressing step may occur at temperature ranging from about 20° C. to about 150° C. In certain embodiments, the dry chitosan solid is dried to a moisture content of less than about 15% (w/w).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE SEVERAL VIEWS OF THE DRAWINGS

FIG. 1. FTIR spectra of CSGelatin1 dried material.

FIG. 2. FTIR spectra of Type Z freeze dried material.

FIG. 3. FTIR spectra of Type Y freeze dried material.

FIG. 4. Image of crosslinked chitosan gelatin (1:4) powder.

FIG. 5. Image of Type Z Catechol modified powder.

FIG. 6. Image of 1st syringe with male Luer screw connector end of example 3 with powder loaded in syringe (no cap).

FIG. 7. Image of 1st syringe and 2nd syringe with female Luer connector (on left with liquid) of example 3 prior to syringes connected to each other.

FIG. 8. Image of 1st syringe and 2nd syringe (on left with liquid) of example 3 with syringes connected to each other by female Luer (on left) and male Luer (on right) screw connector ends.

FIG. 9. Image of 1st syringe and 2nd syringe of example 3 with syringes connected to each other by male and female Luer screw connector ends and with flowable particles substantially dispersed (mixed) uniformly into the liquid without foaming or other signs of gas bubbles.

FIG. 10. Image of 1st syringe with male Luer connector containing uniformly dispersed particle-liquid mixture ready to be delivered.

FIG. 11. Image of 1st syringe containing uniformly dispersed particle-liquid mixture ready to be delivered and connected to a catheter delivery tube for accurate, localized minimally invasive application to an injury.

FIG. 12. Image of deployment of flowable dressing bead from 1st syringe delivery catheter end onto a horizontal, transparent PVC plate with central 4.0 mm diameter hole.

FIG. 13. Image of deployment of flowable dressing bead from 1st syringe delivery catheter end onto a horizontal, transparent PVC plate with central 4.0 mm diameter hole.

FIG. 14. Image of deployment of adhered flowable dressing bead forms from 1st syringe delivery catheter end onto an upright, clear PVC plate with central 4.0 mm diameter hole.

FIG. 15. Image of all flowable dressing (close to 3.5 ml) from 1st syringe delivery catheter transferred to partially cover base of thermoformed polystyrene dish (10 cm×10 cm).

FIG. 16. Images of chitosan flowable dressings (FIG. 16A Type Z; FIG. 16B 1 to 1; FIG. 16C CsGelatin2) adhered to GI tissue sitting in horizontal plane and the same dressings (FIG. 16D Type Z; FIG. 16E 1 to 1; FIG. 16F CsGelatin2) turned upside down.

FIG. 17. Images of chitosan flowable dressings (FIG. 17A Type Z; FIG. 17B 1 to 1; FIG. 17C CsGelatin2) adhered to GI tissue during ex-vivo testing at time=0; the same dressings (FIG. 17D Type Z; FIG. 17E 1 to 1; FIG. 17F CsGelatin2) at 7.8 hours and the dressings; (FIG. 17G Type Z; FIG. 17H 1 to 1; FIG. 17I CsGelatin2) at 19.9 hrs.

FIG. 18. Images of chitosan flowable dressings (FIG. 18A Type Z; FIG. 18B 1 to 1; FIG. 18C CsGelatin2) adhered to Liver tissue sitting in horizontal plane and the same dressings (FIG. 18D Type Z; FIG. 18E 1 to 1; FIG. 18F CsGelatin2) turned upside down.

FIG. 19. Images of chitosan flowable dressings (FIG. 19A Type Z; FIG. 19B 1 to 1; FIG. 19C CsGelatin2) adhered to Liver tissue during ex-vivo testing at time=0; the same dressings (FIG. 19D Type Z; FIG. 19E 1 to 1; FIG. 19F CsGelatin2) at 7.8 hours; the dressings (FIG. 19G Type Z; FIG. 19H 1 to 1; FIG. 19I CsGelatin2) at 19.9 hrs; and the remaining dressings (FIG. 19J Type Z; FIG. 19K 1 to 1.) adhered at 33 hours.

FIG. 20. Images of chitosan flowable dressings (FIG. 20A Type Z; FIG. 20B 1 to 1; FIG. 20C CsGelatin2) adhered to TURP Bladder tissue sitting in horizontal plane and the same dressings (FIG. 20D Type Z; FIG. 20E 1 to 1; FIG. 20F CsGelatin2) turned upside down.

FIG. 21. Images of chitosan flowable dressings (FIG. 21A Type Z; FIG. 21B 1 to 1; FIG. 21C CsGelatin2) adhered to TURP Bladder tissue during ex-vivo testing at time=0; the same dressings (FIG. 21D Type Z; FIG. 21E 1 to 1; FIG. 21F CsGelatin2) at 2.25 hours; the dressings (FIG. 21G Type Z; FIG. 21H 1 to 1; FIG. 21I CsGelatin2) at 6.2 hrs; the dressings (FIG. 21J Type Z; FIG. 21K 1 to 1; FIG. 21L CsGelatin2) at 13.9 hours; the dressings (FIG. 21M Type Z; FIG. 21N 1 to 1.) at 24 hours; the dressings (FIGS. 21O & FIG. 21P Type Z) at 29.5 hours and at 48.7 hours respectively.

FIG. 22 Images of chitosan flowable dressings (FIG. 22A Type Z; FIG. 22B 1 to 1; FIG. 22C CsGelatin2) adhered to esophageal tissue sitting in horizontal plane and the same dressings (FIG. 22D Type Z; FIG. 22E 1 to 1; FIG. 22F CsGelatin2) turned upside down.

FIG. 23. Images of chitosan flowable dressings (FIG. 23A Type Z; FIG. 23B 1 to 1; FIG. 23C CsGelatin2) adhered to esophageal tissue during ex-vivo testing at time=0; the same dressings (FIG. 23D Type Z; FIG. 23E 1 to 1; FIG. 23F CsGelatin2) at 2.25 hours; the dressings (FIG. 23G Type Z; FIG. 23H 1 to 1; FIG. 23I CsGelatin2) at 6.2 hrs; the dressings (FIG. 23J Type Z; FIG. 23K 1 to 1; FIG. 23L CsGelatin2) at 13.9 hours; the dressings (FIG. 23M Type Z; FIG. 23N 1 to 1; FIG. 23O CsGelatin2) at 24 hours.

FIG. 24. Images of Type Z powder (FIG. 24A and FIG. 24B: dry; FIG. 24C and FIG. 24D: wetted with water; FIG. 24E and FIG. 24F: wetted with blood and water).

FIG. 25. Images of CsGelatin2 (FIG. 25A and FIG. 25B: dry; FIG. 25C and FIG. 25D: wetted with water; FIG. 25E and FIG. 25F: wetted with blood and water).

FIG. 26. Images of 1:1 Type Z and CsGelatin2 (FIG. 26A and FIG. 26B: dry; FIG. 26C and FIG. 26D: wetted with water; FIG. 26E and FIG. 26F: wetted with blood and water).

FIG. 27. Images of Type Y powder wetted with blood and water (FIG. 27A and FIG. 27B).

FIGS. 28A-28D. Images of FLOSEAL preparation.

FIGS. 29A-29D. Images of FLOSEAL on stomach, bladder, liver and esophagus.

FIG. 30. Images of FLOSEAL powder using an Amscope T490-DK microscope (FIG. 30A dry; FIG. 30B wetted with water; FIG. 30C wetted with blood).

FIG. 31. Histogram box plots of average post-treatment bleed score and its standard deviation for catechol modified chitosan test materials and HEMOSPRAY (HS) control used in the investigation of hemostasis in porcine parenchymal injuries of heparinized bleeding. The figure provides the hemostatic performance in terms of post-treatment bleed score (low bleed score indicates better hemostasis) of catechol modified chitosan test samples of Type Z, Y, Y1 and Y1_{resid} enabling comparisons to each other and to the positive control of HEMOSPRAY.

FIG. 32. Histogram box plots of average time to hemostasis and its standard deviation for catechol modified chitosan test materials and HEMOSPRAY (HS) control used in the investigation of hemostasis in porcine parenchymal injuries of heparinized bleeding. The best performed catechol chitosan dressings were Y and Y1. According to a two-tailed student t-test, Y and Y1 were statistically equivalent for time to hemostasis in the parenchymal injuries. Hemospray demonstrated a statistically significant longer time to hemostasis than both Y and Y1.

FIG. 33. Histogram box plot of pre-treatment bleed rate for catechol modified chitosan (Y & Y1) and HEMOSPRAY (HS) applications.

FIG. 34. Histogram box plot of time to hemostasis for treatment of gastroepiploic arterial injuries by applications of catechol modified chitosan dressings (Y & Y1) and control HEMOSPRAY (HS).

FIG. 35. Viability of HEE tissues treated with sample extracts including catechol modified chitosan dressing Y3 and HEMOSPRAY control relative to the negative control.

### DETAILED DESCRIPTION

Chitosan endoluminal hemostatic flowable dressing (CEHFD), as used herein, refers to a flowable chitosan dressing that is hemostatic, and can be used in an endoluminal area e.g., inside a resected prostatic fossa to control bleeding or the bladder neck of the resected prostate. CEHFD is not limited by the position of its application and includes chitosan dressing that is applied at any location inside a human body, including but not limited to bladder mucosa.

Bleed rates, or blood flow rates, in ml/min suitable for treatment by the devices described herein may range from about 1 ml/min to about 200 ml/min. In preferred embodiments, the bleeding rates addressed by the devices range from about 1 ml/min to about 150 ml/min. A bleed rate of between about 20 ml/min and 25 ml/min is considered “brisk” bleeding. Oozing bleeding is generally greater than about 1 ml/min as it is noted that low bleeding rates such as 1 ml/min typically clot and stop of their own accord unless the subject is on anticoagulation therapy or has a disorder of the clotting cascade due to reasons other than taking anticoagulation medication. For such a subject with irreversible anticoagulation medication or with a bleeding disorder, 1 ml/min oozing bleeding remains concerning and needs to be addressed such as by the device formed from the material of the invention. In some embodiments, the devices described herein are used to address TURP bleeding rates of between about 1 ml/min and about 25 ml/min, or about 1 ml/min and about 20 ml/min, or about 1 ml/min and about 15 ml/min, or about 1 ml/min and about 10 ml/min, or about 1 ml/min and about 5 ml/min.

In one embodiment, the currently disclosed materials, compositions, and methods are characterized by one or more the following features: (1) an ability to rapidly control hemorrhage in prostatectomy using a noninvasive procedure; (2) an ability to control bleeding in anti-coagulated patients; (3) significantly reduced patient pain and discomfort by control of bleeding without need for prolonged catheterization; (4) significantly reduced hospital length of stay; (5) significantly reduced healthcare cost; (6) significantly reduced rate of morbidity; and (7) trending outcomes to a reduced rate of mortality.

As used herein, bladder mucosa is broadly defined to include any exposed tissue surface in the bladder including any tissue surface exposed by way of an operation (e.g., surgical operation). Bladder mucosa therefore includes bladder mucosa naturally present in the bladder, resected bladder mucosa, and resected prostate, etc.

A TURP delivery device may include any device that is used in a TURP procedure or any device used in connected with a TURP procedure.

### Solid Chitosan Materials

Solid chitosan materials may refer to compositions that include varying amounts of chitosan. The general contents, general chemical compositions and different forms of a chitosan dressings are described, for example, in U.S. Pat. Nos. 7,820,872, 7,482,503, 7,371,403, 8,313,474, 7,897,832, 9,004,918, 8,920,514, 9,204,957, 8,741,335, 8,269,058, 9,205,170, 10086105, and U.S. patent application Ser. Nos. 16/958,301, 16/958,304, 16/958,311, 16/958,307, 16/958,309. Such solid chitosan materials, due to their chemical and physical properties as described previously, have been used to stop bleeding.

The chitosan used preferably comprises the non-mammalian material poly-β-(1-4)-2-amino-2-deoxy-glucopyranose alternately named poly-β-(1-4)-N-acetyl-D-glucosamine. The chitosan can be processed in conventional ways from chitin obtained, for example, from sources including and not limited to fungi, diatoms, and crustacea such as shrimp. Chitosan may be biocompatible and biodegradable within the body, and is capable of being broken down into glucosamine, a benign material. The catechol-modified chitosan used herein may include reference to catechol-added chitosan.

The solid chitosan material may be dry or wet. The solid chitosan material is “dry” if the moisture content in the chitosan is less than about 15% by weight, preferably about 10% by weight, and more preferably about 5% by weight. A chitosan dressing is “wet” when the chitosan dressing has come in contact with a source of water, including water in a physiological environments and biological fluids, or in an aqueous solution. For example, the solid chitosan material of the disclosure is initially wetted when the solid chitosan, as described in this disclosure, is mixed into its flowable composition and which subsequently comes into contact with gastrointestinal tract fluid, urine, or blood or a tissue surface of gastrointestinal tract or bladder (bladder mucosa or GI mucosa). The solid chitosan material, remaining substantially in a solid form in the flowable dressing absorbs, displaces, redirects or channels water/moisture in the physiological environment of gastrointestinal tract or bladder mucosa in amounts sufficient to permit adhesion of the solid chitosan material to the tissue surface. The adhered chitosan material can be used to seal wound surfaces and slow or stop further bleeding.

In a preferred embodiment, the solid chitosan material is a reduced particle component of the chitosan gastrointestinal flowable hemostatic dressing (CGHFD) or chitosan endoluminal hemostatic flowable dressing (CEHFD) formed from the material described herein. In a preferred embodiment, the solid chitosan material contains preferably greater than or equal to 25% by weight chitosan; more preferably greater than or equal to 50% by weight chitosan and most preferably greater than or equal to 75% by weight chitosan. Chitosan is a generic term used to describe linear polysaccharides that are composed of glucosamine and N-acetyl glucosamine residues joined by β-(1-4) glycosidic linkages (typically the number of glucosamines≥N-acetyl glucosamines) and whose composition is soluble in dilute aqueous acid (Roberts 1992[24]). The chitosan family encompasses poly-β-(1-4)-N-acetyl-glucosamine and poly-β-(1-4)-N-D-glucosamine with the acetyl residue fraction and its motif decoration (either random or block) affecting chitosan chemistry. The C-2 amino group on the glucosamine ring in chitosan allows for protonation, and hence solubilization of chitosan in water (pKa≈6.5) (Roberts 1992 [25]).

In a preferred embodiment of CGHFD, the solid particle component of the flowable dressing formed from the material of the invention is polymeric, biocompatible, tissue adherent and hemostatic.

In a preferred embodiment of CEHFD, the solid particle component of the flowable dressing formed from the material of the disclosure is polymeric, biocompatible, tissue adherent and hemostatic.

In some CGHFD embodiments, the most common gastroscope channel is 0.28 cm diameter (2.8 mm) and hence this is the most preferred size for the flowable dressing delivery. Alternatively, a more preferred size is 0.32 cm diameter, which is a standard gastroscope channel diameter but less common than the 0.28 cm channel. Another preferred gastroscope channel diameter size is between 0.45 cm and 0.32 cm which is more a custom gastroscope channel size and, thus, less common than the 0.32 or the 0.28 cm gastroscope channel diameter size.

In a preferred embodiment of CEHFD the flowable dressing is delivered to the resected prostate fossa by a balloon catheter with a delivery port located between distal and proximal balloons. In a preferred embodiment of the delivery catheter, there is an expandable region of the catheter between the proximal and distal balloons that may be expanded to apply pressure over the flowable dressing volume that has been delivered into the cavity of the resected fossa. The purpose of the distal and proximal balloons is to locate and isolate the flowable dressing port over the resected fossa cavity and possible injury in the bladder neck so that the delivered volume of flowable dressing will fill the resected fossa cavity up to the region of the bladder neck to provide local control of bleeding. In one embodiment, the channel size for CEHFD not limited by use of endoscope but limited by typical urological catheter sizes (16-24 Fr. 1Fr=0.33 mm) and number of connecting channels available in catheter (up to 5 with main channel being for irrigation around 12 Fr).

In a preferred CEHFD balloon catheter embodiment, the delivery catheter internal diameter is greater than 1.5 mm. Alternatively, a more preferred delivery catheter internal diameter is greater than 1.8 mm. Alternatively, a most preferred delivery catheter internal diameter is greater than 2.4 mm.

A flowable dressing as described herein is able to be readily delivered by catheter through an endoscope channel, is not readily soluble in blood or body fluid, such as GI fluids or urine, at about 37° C. within, preferably, the first 6 hours of application, more preferably the first 12 hours of application, and most preferably the first 24 hours of application, and degrades and/or dissolves fully in contact with GI fluids or bladder fluids at about 37° C. within about 7 days.

A flowable dressing as described herein will not adhere to the delivery device or clog the delivery catheter, it does not swell or shrink appreciably, i.e., it does not increase or decrease in size by more than about 25% in volume in the presence of blood and body fluids (GI fluids or urine or bladder fluids or mixture thereof) at about 37° C.

In a preferred embodiment, the dressing may be terminally sterilized without affecting dressing characteristics. When it is stored under controlled conditions in its packaging at room temperature of about 21° C. to about 25° C., its tissue adhesion properties, mechanical properties, dissolution properties in GI or bladder fluids, swelling properties, and hemostatic properties are stable and do not change appreciably over time (e.g., about ≤2 years).

A preferred embodiment of the flowable dressing is that immediately prior to use and mixing, it is formed of a substantially dry chitosan powder composition and a separate (before mixing) carrier liquid fluid. The water content of the dry chitosan powder composition before mixing is about ≤15% by weight, or about ≤8% by weight. The dry chitosan composition is preferably a powder formed by a spray drying process or by reduction of chitosan solid dry material. The solid dry chitosan material may include but not be limited to a course granule, a sheet, a membrane, a film, a ribbon, a web, a rod, a fibrous mat, and a fiber.

In a preferred embodiment of the chitosan solid dry material, the flowable dressing preparation process may include grinding a material produced from a compression process that changes the solid chitosan material density from an initial preferred range of about 0.005 g/cm³ to about 0.05 g/cm³ to a final preferred range of about 0.03 g/cm³ to about 0.7 g/cm³; however, ranges of about 0.08 g/cm³ to about 1.2 g/cm³ are also contemplated. It is noted that a density of about 1.5 g/cm³ is the density of void-free chitosan solid material. The compression process may include application of temperature in the range of about 20° C. to about 150° C. To avoid substantial dressing swelling of the dry compressed dressing on contact with biological fluid, the temperature of the compression is preferably applied by a method that may include but not be limited to convection, conduction and radiation, and the temperature of the compressed chitosan material should preferably be maintained at least about 80° C. for at least about 15 seconds.

Heat during compression is a tool that allows plasticization and molding of the dry chitosan solid material without cracking or tearing of the chitosan (non-destructive molding). The first glass transition temperature (Tg) of pure dry chitosan is near 80° C. which if processed near in the case of pure dry chitosan will allow ready non-destructive molding of the chitosan as well as some crystalline annealing of its structure. It is possible to lower the Tg by application of plasticizers such as water or glycerol to the chitosan and hence provide a similar level of non-destructive molding at lower temperature. Here, it is noted that chitosan can be molded non-destructively in the range 20° C. to 150° C. Outside of this range it would still be possible to non-destructively mold the chitosan but would be much more difficult. Above 150° C. the chitosan begins to thermally degrade while below 20° C., the addition of plasticizers may lead to undesirable loss of chitosan crystallinity which provides for dissolution resistance and resistance to degradative processes such as occur in sterilization.

Preferably, the compression prevents substantial swelling of the dry compressed chitosan solid on contact with biological fluid and is performed with moisture content of the dry dressing during the compression at about ≤15% w/w. The compression may be applied through twin or multi-roller compression and/or uniaxially between adjacent platens.

The compression may be against a flat surface. Alternatively, the compression may be applied against an etched, machined, ablated or other type of surface treatment that imparts a depleted or added surface texture. The surface texture may be a random or it may be a regular repeated pattern.

Preferred embodiments of the biocompatible, bio-dissolvable, tissue adherent chitosan flowable dressing are able to resist dissolution in gastrointestinal (GI) fluid and blood at about 37° C. for at least about 6 hours, are tissue adherent, and include materials and material structures that promote resistance to rapid dissolution and degradation in the low pH and strongly enzymatic digestive fluid of the upper gastrointestinal tract. This is a significant advantage of the chitosan flowable dressings disclosed herein because the upper gastrointestinal digestive tract has evolved to rapidly digest most organic materials including chitosan, cellulose and starch.

Preferred embodiments of the biocompatible, bio-dissolvable, tissue adherent chitosan flowable dressing are able to resist dissolution in bladder fluid and blood at about 37° C. for at least about 6 hours is tissue adherent and includes materials and material structures that promote resistance to rapid dissolution and degradation in urine of the bladder. This is a significant advantage of the chitosan dressings disclosed herein.

Chitosan flowable dressings provided herein can be applied to a mucus surface, e.g., in GI or the bladder upon contact or by light contact on a tissue surface to interact to promote adherence with the injury site to stop bleeding.

### Production of Chitosan Flowable Dressing Particles

The solid powder of the chitosan dressings of the present invention may be generated using various reduction methods and processes. Such reduction method and processes may include but not be limited to granulating (larger solid forms into course granules>1 mm in diameter), milling (converting granules to powders<0.5 mm in diameter), grinding (reduction in powders to <0.2 mm in diameter), sieving (to select for particle size), spray drying (direct forming of small solid particles from liquid without milling or grinding), and chopping (of micron and sub-micron diameter fiber to produce chopped, low aspect ratio fiber of length: diameter<200:1). It is to be understood that the reduced particle forms of the disclosure remain as solid materials in both their dry or their wet flowable configurations. In the presence of liquid, the particles may be present as solid and semi-solid material. The solid particle forms of the disclosure may swell in volume (<50%) but remain solid in the presence of liquid. These solid materials remain as discrete individual particles and they retain their essential solid property by resisting both dissolution and deformation. The semi-solid particle forms of the disclosure may swell in volume (≥50%). These semi-solid materials remain as discrete individual particles and they retain their essential semi-solid property by resisting dissolution while demonstrating swelling deformation in shape. The dry semi-solid particle may have an original appearance of a course, sharp edged grain which when exposed to liquid becomes enlarged and swollen to a more rounded, possibly spherical shape.

A preferred form of milling and grinding is at or below −40° C. such as in the presence of dry ice. A more preferred form of milling and grinding is at cryogenic temperature at or below −180° C. such as in the presence of liquid argon or liquid nitrogen. A preferred cryogenically cooled milling system is one that incorporates a screen within the mill that selects for particles during the milling process with the preferred larger particle radius selected and minimization/elimination of occurrence of fine particles (any particle<10 microns radius of gyration). Alternatively, fine particles created during milling may be removed by sieving of the milled powder, but this comes with reduced yield compared to a screened milling process. The presence of a significant level of fine particles in a flowable dressing particle dispersion directly interferes with ability of blood and its component cells to permeate through the interconnected pore structure of the flowable dressing once it is deposited over an injury and thus significantly impair the flowable dressing's ability to achieve rapid and reliable hemostasis. In one embodiment, porosity is uninterrupted in the flowable dressing with interconnected pore size range of 10-100 microns with substantially most of the pores near 10-50 microns. The uninterrupted pore structure is indicated in the flowable dressings by their ability to absorb biological fluid such as blood.

In some embodiments of CGHFD, the chitosan flowable dressing provided herein, due to its compositional structures and characteristics, can be delivered to stop bleeding. In some embodiments, the chitosan dressing provided herein, therefore, is able to be delivered through a narrow working channel. Exemplary diameters of a narrow working channel through which the chitosan dressing provided herein can be delivered include a diameter of about 3.2 mm or less, and including, but not limited to, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, and 3.2 mm.

In some embodiments of CEHFD, the chitosan flowable dressing provided herein, due to its compositional structures and characteristics, can be delivered to stop bleeding. In some embodiments, the chitosan dressing provided herein, therefore, is able to be delivered to an injury and injury sites by balloon catheter through one or more delivery channels. Exemplary diameters of a delivery channel through which the chitosan flowable dressing provided herein can be delivered include a diameter of about 3.2 mm or less, and including, but not limited to, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, and 3.2 mm.

### Catechol Modified Chitosan; and Its Production

The chitosan solid dry chitosan materials described herein relate to chitosan comprising catechol modified chitosan and/or hydrophilic polymers. Other aspects of chitosan dressing comprising catechol modified chitosan are described in more detail below.

Preferred embodiments of the CGHFD or CEHFD of the disclosure include compositions with catechol modified chitosan and/or, optionally, other hydrophilic polymers. Preferably the catechol modified chitosan in the dressing provides prolonged adherence to wetted tissue with tissue adherence≥about 1 kPa resisting dissolution in water, saline solution, blood and/or GI or bladder fluid at about 37° C. for ≥about 6 hours.

A preferred embodiment of the invention is that the catechol modified chitosan may be formed by N-acylation of the C-2 amine on the chitosan glucosamine by 3,4-dihydroxyhydrocinnamic acid (alternatively named 3-(3,4-Dihydroxyphenyl) propionic acid, Hydrocaffeic acid)). Alternatively, the chitosan N-acylation to produce a catechol modified chitosan may include but is not limited to a modification with one of a 3,4-Dihydroxycinnamic acid (caffeic acid); a trans-3,4-Dihydroxycinnamic acid (trans-caffeic acid); and a 3,4-Dihydroxyphenylacetic acid (DOPAC, Homoprotocatechuic acid).

A preferred embodiment of the invention is that the catechol modified chitosan may be formed by N-acylation of the C-2 amine on the chitosan glucosamine and the amine on 3,4-Dihydroxyphenethylamine (alternatively named dopamine) with carboxylic acid groups on a tricarboxylic acid including citric, isocitric and aconitic acid.

A preferred embodiment of the invention is that the catechol modified chitosan may be formed by reductive alkylation of the C-2 amine on the chitosan glucosamine by addition of 3,4-dihydroxybenzaldehyde to the chitosan with reduction of the intermediate imminium functional group using sodium cyanoborohydride or sodium borohydride.

The presence of catechol in the composition provides for some poly-conjugated structure as the catechol is oxidized to o-quinone. This causes visible difference between the unmodified chitosan and catechol modified chitosan compositions, which may be off-white or pink to dark brown and black in color, respectively. It is noted that the catechol modified chitosan compositions go from pink to black when oxidation occurs in the catechol.

Pink coloration in the catechol modified chitosan, signifying substantial absence of crosslinking, is provided in the aqueous synthesis by maintaining pH reaction solution at or below pH 5.5. The pink coloration may also be provided in the aqueous synthesis by performing the modification and subsequent processing steps substantially in the absence of oxygen such as by using aqueous systems purged with an inert gas which may include but not be limited to argon or nitrogen. Although the pink coloration is not desirable in the final solution or catechol modified product, it may be desirable in intermediate handling stages (such as immediately after chitosan derivatization with catechol and/or dialysis and/or washing of the subsequent catechol chitosan solution to remove residual unreacted material) because it allows for stable dry product polymer storage and dry product weight determination with subsequent ability to substantially re-dissolve the pure dry catechol modified product in water to a desired dry weight at a later time. This water-soluble chitosan catechol material is then subsequently oxidized and crosslinked (with brown or darker coloration).

In a preferred embodiment, the catechol modified chitosan is not removed from solution by an intermediate drying step to allow for storage but rather it is kept in aqueous solution and oxidized in aqueous solution by exposure to higher than about pH 5.5 in the presence of atmospheric oxygen. Preferred pH control is achieved by adjustment of partial pressure of aqueous dissolved carbon dioxide (increased partial pressure reduces pH while decreased partial pressure increases pH to nearer pH 7). An alternative preferred means of pH control is by incremental addition of a strong acid to lower pH and a strong base to raise pH. Examples of strong acids may include, but are not limited to, hydrochloric acid, sulphuric acid and nitric acid. Examples of strong bases may include but not be limited to sodium hydroxide and potassium hydroxide. Subsequent drying of this aqueous water-soluble oxidized catechol modified chitosan results in a preferred level of crosslinking of the catechol chitosan with good resistance to dissolution and degradation in the upper gastrointestinal tract and in bladder. The catechol chitosan solution may be diluted by addition of water or concentrated by water removal. The water may be removed by the techniques including, but not limited to, ultrafiltration, reverse dialysis and centrifugation. The solid fraction of the solution may be determined by sampling a known volume from the solution and performing analyses including but not limited to gravimetry, Fourier transform infrared spectroscopy, ultraviolet-visible spectroscopy, refractometry, and pycnometry.

In a preferred embodiment, the catechol modified chitosan composition is of a brown to darker color resulting from catechol oxidation to o-quinone. The quinone is produced by autoxidation of the catechol hydroxyls in the presence of oxygen and at pH above about 5.5. Schiff base reaction of quinone with chitosan C-2 amine produces crosslinking in the modified chitosan. The color of the catechol modified chitosan composition is controlled during synthesis by controlling pH and oxygen exposure. Maintenance of pH at or below about pH 5.5 inhibits the production of o-quinones. Subsequent conditioning of dialysis solution, final washed, or dialyzed catechol chitosan solutions in a preferred pH range 5.8 to 6.2 provides for more dissolution resistant, darker, more oxidized catechol. In some embodiments, the coloration of catechol modified chitosan characterizes one aspect of the catechol modified chitosan dressing. In some embodiments, the coloration reflects the degree of substitution of the chitosan with catechol. In some embodiments, the coloration from pink to brown to darker color correlates with the degree of substitution.

In a preferred embodiment, the solid particles of the flowable chitosan dressing of the invention are formed of a catechol chitosan composition that is iron enhanced. The addition of Fe(III) salt, such as addition of FeCl₃, to a catechol modified synthesis of chitosan with a molar ration of Fe(III) to catechol reactive species, such as 3,4-Dihydroxyhydrocinnamic acid, of between 1:1 and 1:5 results in significantly improved efficiency of the reaction with ability to achieve fractional degree of substitution of the catechol reactive species covalently attached to the chitosan up to around 0.7.

In order to prepare the dry powder of the flowable dressing from the catechol chitosan, a preferred light brown to darker brown to a black catechol aqueous chitosan solution is prepared which may be used by itself or may be mixed with other aqueous hydrophilic polymer solutions including but not limited to solutions of chitosan and/or, optionally, hydrophilic polymers. Preferably, the dry phase separated catechol chitosan particles and powders of the flowable dressings are prepared from densified dried structures.

Preferred crosslinked catechol modified chitosan compositions of the invention provide good tissue adherence and 10 times to 100 times increased resistance to dissolution in the upper gastrointestinal tract or bladder compared to dressings formed substantially of unmodified chitosan. The catechol modified chitosan compositions described herein, provide flowability, longevity, biocompatibility, and ability to eventually dissolve.

Preferred rapid adherence to gastrointestinal mucosa or bladder mucosa of CGHFD or CEHFD formed from the material of the invention (≤1 minute) is provided in the flowable chitosan dressing by the promotion of quaternary ammonium cation formation at the chitosan glucosamine C-2 amine by the presence of an acid in the dry dressing composition or naturally present in the biological environment such as in the upper gastrointestinal tract. Preferred chitosan acid salts in the dressing may include salts of acetic, lactic, glycolic, citric, succinic, malic, hydrochloric, glutamic, ascorbic, malonic, glutaric, adipic, pimelic, and tartaric acids, and combinations thereof. Preferably the acid salt % weight of the chitosan is greater than about 2% and less than about 15%. To achieve fast adherence (e.g., ≤1 minute) to wet tissue, the moisture in the dry gastrointestinal or bladder solid chitosan particle before mixing with carrier diluent is preferably less than about 15% by weight; more preferably it is less than about 10% by weight and most preferably it is less than about 5% by weight.

In the case of solidified, freeze-phase-separated and sublimated (to remove ice) chitosan materials used as precursor solid sheet material to prepare dried powder, the chitosan solution is poured into the freeze-phase-separation mold (typically in the shape of a pan with a horizontal flat base) with preferably around a 0.25% w/w, more preferably around 0.5% w/w and most preferably 1.0% w/w hydrophilic polymer chitosan solution. The hydrophilic polymer solution is preferably added to the horizontal flat pan to a vertical depth of preferably about 5 mm, more preferably 8 mm and most preferably 12 mm mold depth. The solution in the mold is subsequently frozen and dried to remove water by sublimation or freeze phase substitution (solvent extraction of the ice with a non-solvent to the polymer) to a low density (>99% void volume) open or porous dry sponge with a dry density<about 0.01 g/cm³ (or, for example, about 0.005 g/cm³ for a catechol chitosan uncompressed dressing from 0.5% solution, which is about ⅕ or 20% of the density of an uncompressed HemCon Bandage chitosan sponge, which is about 0.025 g/cm³). Lyophilization is typically performed at pressure below 300 mTorr while freeze substitution involving a dry, cold (e.g., <−20° C.) solvent such as ethanol is performed at atmospheric pressure. The dry sponges may be compressed to greater than about 0.4 g/cm³ density and less than about 100 microns thickness. The compression is not limited to but may include uni-axial compression between aligned flat platens, wherein the platens are heated between 18° C. and 150° C. and are pressure loading up to 10,000 bar.

The compression creates a thin (e.g., range from about ≤100 microns to about ≤500 microns) strong (e.g., 5 MPa to 25 MPa UTS) chitosan sheet that may be ground and/or milled to form a final dried chitosan powder component of the flowable dressing.

Freeze phase separation of dilute aqueous polymeric solutions results in phase separation of micron and submicron thin polymeric chitosan lamella interspersed regularly between ice crystal sheets close to 200 microns in width. Removal of the ice by sublimation (freeze drying) or alternatively by solvent extraction leaves the dry sponge composed of close-to-aligned, thin (≤1 micron), polymeric chitosan lamella. Compression of the polymeric chitosan lamella at close to or greater than their glass transition temperature (Tg for dry chitosan is near 80° C.) allows for their compression into the thin (near 100 microns) dense polymeric structure formed of layers of hundreds of strong compliant polymeric chitosan leaves (lamella) which do not readily propagate cracks and which can be folded repeatably without failure. Such multi-leaf layering achieves remarkable strength. Prior to the present invention, no one has previously investigated freeze-phase-separated chitosan sheets for manufacture and use as described herein and with the aim to address key problems solved by the present invention such as, for example, flowable material adhesion by removal of interfering fluids (by absorption, channeling, displacement, and/or re-direction), ability to be readily delivered to control hemorrhage.

In order to prepare one dressing from two sheets with the dressing having a catechol chitosan tissue adhesive surface layer and an unmodified chitosan surface layer, two sheets (one of catechol modified chitosan and the other of chitosan acid salt) are bonded together by, for example, placing one sheet on top of the other and applying sufficient uniform pressure over the dressings to compress them to a higher density. In a preferred process, the original densities of each sheet type at ≤about 0.03 g/cm³ is increased to a final dressing density≥about 0.30 g/cm³. In a more preferred process, the original densities of each sheet type at ≤about 0.015 g/cm³ is increased to a final dressing density≥about 0.4 g/cm³. In a most preferred process, the original densities of each sheet type at ≤about 0.01 g/cm³ is increased to a final dressing density≥about 0.5 g/cm³. At the conclusion of the compression, the two compressed sheets are bonded together so that one cannot be readily peeled away from the other and the dressing can be manipulated by folding and furling without any occurrence of separation.

This physical adherence of materials by compression of two or more low density porous materials together to form a final two or more layer porous material of higher density solves a difficult problem of how to adhere such materials together without physical or chemical change to the individual materials and without addition of further bonding agents or adherents. It is contemplated that bonding may be attributed to microsurface impingement and penetration of the dressings through their pores with physical interlocking due to pore compression. This physical interlocking of low density, freeze phase separated, dry sheets is not restricted to two materials of the same thickness or to only two layers since the interlocking effect is neither sidedness nor thickness dependent. Therefore a multi-layered sheet construct of individual freeze phase separated and dried sheets of the same or different materials of the same or different thickness may be formed by layering the low density sheets (preferably with density≤0.05 g/cm³) and compressing the assembly together to a density≥0.3 g/cm³). Such a final physically adhered assembly would be expected to provide advantages of thin top and bottom surface layers including but not limited to adhering or anti-adhering materials with layers inside providing including but not limited to structural, physical and chemical elements. A preferred powder of the flowable hemostat of the invention may be formed by grinding and/or milling a multi-layered sheet.

In one mechanism, the flowable chitosan dressing provided in this disclosure is able to stop bleeding by absorbing, channeling, and/or redirecting the hydrophilic and hydrophobic fluids at an injury site. The absorption clears enough moisture from the injury site to allow subsequent hemostatic reactions between the chitosan dressing and the tissue at the injury site, which in turn stops bleeding and allows the chitosan dressing to stay attached; thus, sealing the injury site. The porous, dense, and multi-layer structure of the particles of the flowable chitosan dressing provided herein facilitates the absorption, channeling, and/or redirection of the moisture at the injury site, and the attachment or adherence of the flowable chitosan dressing to the injury site.

The chitosan dressing disclosed herein is biocompatible. In some embodiments, the dissolved residue from a chitosan dressing applied to an injury site in vivo passes safely through the alimentary tract or urethra and is excreted along with other bodily waste.

More than one, or multiple, flowable chitosan dressings may be used or applied in serial fashion to a tissue treatment site or injury site. When more than one chitosan dressing is deployed, such dressings may separately adhere to adjacent tissue site or injury site areas, or may overlap with each other to varying extents. Due to the economy of material use of the flowable chitosan dressing described herein, depending on the application, it is contemplated that multiple chitosan flowable dressings may be used as needed to promote or achieve hemostasis of an injury site.

In one embodiment, the chitosan flowable dressings overlap one another upon application. In such an instance, ideally there would be some adherence of the wetted adhesive side of the subsequent dressing to the wetted top dressing surface of the earlier dressing. Accordingly, the chitosan flowable dressing has a top surface that provides for sufficient adherence for placement of subsequent overlapping chitosan flowable dressings.

### Delivery Device

A delivery device, as used herein, is a device for delivering chitosan dressing. A delivery device delivers a flowable chitosan dressing to injury sites at different locations in the body of an animal including, but not limited to, humans, pigs, dogs, etc.

In some embodiments, a delivery device is a minimally invasive device that can deliver a dressing, e.g., a chitosan flowable dressing, to a physiological site in the body of an animal, in non-invasive or minimally invasive manner. In some embodiments, the delivery device is a catheter. In some embodiments, the non-invasive or minimally invasive feature of the delivery device is achieved through delivery of a flowable dressing, e.g., a chitosan flowable dressing, through a narrow catheter or a comparable working channel. In some embodiments of CGHFD, the catheter or the comparable working channel has a diameter that is less than 3.2 mm. In other CGHFD embodiments, a gastroscope channel may range in diameter size from 2.8 mm to 4.5 mm. In some CEHFD embodiments, the balloon catheter or the comparable working channel has a diameter that is less than about 7 mm. In other CEHFD embodiments, a channel of a TURP delivery device may range in diameter size from 0.5 mm to 4.0 mm.

In some CGHFD embodiments, the catheter or balloon catheter or the comparable working channel has a diameter that is less than 3.2 mm. In other CGHFD embodiments, a gastroscope channel may range in diameter size from 2.8 mm to 4.5 mm.

In some CEHFD embodiments, the catheter or balloon catheter or the comparable working channel has a diameter that is less than about 7 mm. In other embodiments, a channel of a TURP delivery device may range in diameter size from 0.5 mm to 4.0 mm.

Exemplary delivery devices include, but are not limited to, a balloon device, a balloon catheter, an indwelling catheter, a urethral or suprapubic catheter, an external catheter, a short-term catheter, and an intermittent catheter.

A delivery device can also be an endoscopic device used in various aspects of medical procedures. In some embodiments, the endoscopic device is non-invasive or minimally invasive due to a narrow catheter or tube/tubing or a similarly narrow-diameter portion of the device.

A delivery device can also be a transluminal or transurethral delivery device. In some embodiments, the transurethral delivery device is non-invasive or minimally invasive due to a narrow catheter or tube/tubing or a similarly narrow-diameter portion of the device.

Delivery devices include other devices with narrow-diameter tubing, channels, or catheters, or similar structures.

The flowable dressing chitosan material described herein can be delivered accurately as a final fluidized dispersion to a remote injury site by, for example, catheter delivery to adhere to the tissue of the remote site independent of gravity to quickly effect bleeding control, close the injury site and stay in place resisting dissolution for up to and more than 6 hours. The flowable dressing chitosan material of the disclosure adheres on contact, and for greater than 6 hours, to mucosal tissue and tissue injury sites. The flowable chitosan material of the disclosure may be applied upside down under normal gravity to adhere to an injury site with endoscopic application without loss of coverage or without flowing away from its site of application. After 6 or more hours of application to an injury site, the top portion of the applied flowable dressing chitosan material may be eroded or bio-dissolved but the flowable dressing material closest to the injury remains adhered as a thin, uniform layer covering the injury site for at least 12 hours to protect the injury site and reduce opportunity for rebleeding. The flowable dressing of the disclosure may be used with minimally invasive techniques for remote dressing delivery to quickly deploy dressing to achieve hemostasis, to fill and to close resections, biopsy sites, narrow recesses, defects and openings around hemostatic clips, sutures, clamps, staples, wires and pins.

### Applications and Methods of Treatment

The chitosan flowable dressing embodiments formed from the material of the invention provided in this disclosure may be used to stop bleeding in suitable diseases, conditions, disorders, or emergent traumas or injuries. In some embodiments, the dry solid chitosan material of the invention may be used to stop bleeding from any wet physiological surface, e.g., mucus. Exemplary applications include, but are not limited to, gastrointestinal tract or bladder bleeding, other intraluminal applications, including vascular applications, internal surgical bleeding, internal biopsy bleeding, internal bleeding following parenchymal organ resection, and oral, ocular, auditory or nasal bleeding. Additional applications that might require addition of water or fluid to encourage adhesion of the chitosan dressing to a tissue surface or injury site are also contemplated, for example, use of the chitosan dressing on external body surfaces.

The chitosan flowable material of the present invention may be used for treatment of gastrointestinal bleeding that may include but not be limited to treatment of bleeding in esophageal varices, bleeding from peptic ulcers, bleeding from duodenal ulcers, bleeding associated with biopsy of the upper and lower gastrointestinal tracts, resections of the upper and lower gastrointestinal tracts, and tears or ruptures in the upper and lower gastrointestinal tracts. Other diseases, conditions, disorders, or emergent traumas or injuries may include, but are not limited to, internal arterial injury; internal bleeding from the liver, internal bleeding from the vena cava; injury in the thoracic cavity including perforations of the heart and lungs and their vessels; and injuries of the abdominal cavity.

The chitosan flowable material of the present invention may also be used for treatment of transurethral prostatectomy and or bladder neck bleeding that may include but not be limited to treatment of bleeding.

The chitosan material of the present invention may also be used following acute internal injury (such as occurring in UGIB, TURP or other minimally invasive procedure or in open surgery) to protect the injured site by closing the site of injury and providing an environment conducive to cellular regeneration before dissolving or degrading within 7 days.

In a preferred embodiment, the flowable dressing comprises an optical contrast material that provides enhanced endoscopic visualization of a deployed flowable dressing for improved wound placement and post placement observation of the wound and dressing. The material can be mixed readily with the composition of the flowable dressing and delivered with the flowable dressing to address bleeding and provide for enhanced ability to visualize the dressing edges, the body of the dressing and successful hemostasis. The enhanced visualization material remains bound and uniformly present within the flowable dressing composition without any significant leakage of the material into the biological environment while the flowable dressing is providing hemostasis. Enhanced visualization materials may include but not be limited to fluorescent agents, quantum dots, nanoparticles containing fluorescent agents, chitosan covalently modified with a fluorescent agent, gold nanoparticles, organically modified dye-doped silica, upconverting phosphors and lanthanide-based contrast agents.

In a preferred embodiment, the flowable dressing hemostasis may be augmented by endoscopic placement of a supportive solid mesh or dressing over the flowable dressing once the flowable dressing has been deployed over a bleeding site. In the case of strong arterial bleeding which may prove too high a pressure for the cohesive strength of the flowable dressing to immediately control bleeding due to tunneling or other penetration of the arterial bleeding pressure through the flowable dressing, then application of light tamponade pressure for a short period by balloon or similar endoscopically applied basket device through an intermediary dressing or mesh material over the flowable dressing. The application of the support dressing or mesh with light pressure closes any tunneling or other penetration within the flowable dressing and provides for prolonged hemostasis under difficult bleeding conditions.

The chitosan flowable material of the present invention may also be used to be delivered to, and to locally adhere to, specific target sites for general therapeutic purposes including active pharmaceutical agent and/or biological agent delivery. Such target sites would include but not be limited to anastomoses, esophageal varices, peptic ulcers, resected prostatic fossa, resections and biopsies of the liver, resections and biopsies of the kidney, resections and biopsies of the bladder, resections and biopsies of the throat, resections and biopsies of the pancreas, resections and biopsies of the stomach, resections and biopsies of the lower gastrointestinal tract, resections and biopsies of the lung, and resections and biopsies of the heart.

The dissolution resistant, tissue adherent, flowable hemostatic dressing of the invention may be applied to an injury site to remain in place, control bleeding and close openings such as fistulas by promotion of tissue growth over the opening.

The dissolution resistant, tissue adherent, flowable hemostatic dressing of the invention may be used in transarterial embolotherapy by delivery inside a blood vessel to effect local closure of the blood vessel. Transarterial embolotherapy is a local therapy to obstruct blood flow to tumors to moderate and/or eliminate their growth.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

Examples 1-12 are drawn to chitosan endoluminal hemostatic flowable dressing (CEHFD) and chitosan gastrointestinal hemostatic flowable dressing (CGHFD) devices for flowable, hemostatic, dissolution-resistant, tissue adherent and persistently tissue adherent applications.

The following materials and preparations were considered in the chitosan endoluminal hemostatic flowable and chitosan gastrointestinal hemostatic flowable dressing inventive process.
Chitosan A: Primex CHITOCLEAR 65010, TM 4375, MW=250-300 kDa, Brookfield
Chitosan B: Primex CHITOCLEAR 43000, TM 4167, MW=110-150 kDa, Brookfield viscosity in 1.0% w/w chitosan solution in 1.0% acetic acid at 25° C. and spindle LV1=9 cPs, DDA=95% (by colloidal titration).
Glacial acetic acid: Fisher Scientific, Catalogue No. A38-212.
Hydrochloric acid: 1.0 M aqueous solution Sigma Aldrich, Catalogue No. H9892.
L-Lactic acid: JT Baker, Catalogue No. 0196-01.
Glycolic acid: JT Baker, Catalogue No. M821-05.
Sodium hydroxide: 5.0 M NaOH aqueous solution Sigma Aldrich, Catalogue No. S8263-150 ml.
Potassium hydroxide: 0.1 M KOH in methanol (BDH).
Ethanol: 200° Proof Sigma Aldrich, Catalogue No. 459844-1L.
Acetic anhydride: ACS reagent grade obtained from Sigman Aldrich, Catalogue No. 320102-1L.
Sodium cyanoborohydride: Sigma Aldrich Catalogue No. 8180530250
Sodium borohydride: Sigma Aldrich Catalogue No. 452882-500G
N, N-dimethylformamide: Sigma Aldrich Catalogue No. 227056-2L
Deionized water: Sigma Aldrich Catalogue No. 8483339010
3,4-Dihydroxyhydrocinnamic acid (hydrocaffeic acid): 98% Sigma Aldrich, Catalogue No. 102601.
3,4-Dihydroxycinnamic acid (caffeic acid): 98% Sigma Aldrich Catalogue No. C0625
trans-3,4-Dihydroxycinnamic acid (trans-caffeic acid): Sigma Aldrich Catalogue No. 51868
3,4-Dihydroxyphenylacetic acid (DOPAC, Homoprotocatechuic acid): 98% Sigma Aldrich Catalogue No. 850217
3,4-dihydroxybenzaldehyde: Sigma Aldrich Catalogue No. 8204750100
3,4-Dihydroxyphenethylamine (DOPA, dopamine): Sigma Aldrich Catalogue No. H8502-100G
1-ethyl-3-(-3-dimethylamino-propyl)-carbodiimide: (alternatively N-(3-dimethylaminopropyl)-N′-ethylcarbodiimide hydrochloride with common acronym EDC) Sigma Aldrich Cat. #E7750
N-hydroxy succinimide (NHS): Sigma Aldrich Catalogue No. 8045180100
Citric acid: Sigma Aldrich, Catalogue No. C0759-1 KG
Sodium Chloride Sigma Aldrich, Catalogue No. 793566-500 g.
Poloxamer 407: Spectrum, Catalogue No. P1166.
Synthetic urine formulation: Add calcium chloride dihydrate Sigma Catalogue No. C5080 (1.30 g); magnesium chloride hexahydrate Sigma Catalogue No. M2670 (1.30 g); sodium chloride Sigma Catalogue No. 793566 (9.20 g); sodium sulphate Aldrich Catalogue No. 238597 (4.60 g); sodium citrate dihydrate Sigma catalogue No. S4641 (1.30 g); sodium oxalate Sigma Catalogue No. 71800 (0.04 g); potassium dihydrogen orthophosphate Sigma Catalogue No. P5655 (5.60 g); potassium chloride Fisher Catalogue No. BP366 (3.20 g); ammonium chloride Sigma Catalogue No. 09718 (2.00 g); urea Sigma catalogue No. U1250 (50.00 g); creatinine Acros Catalogue No. 228940500 (2.20 g) to a 2.0 liter volumetric flask and add 1.5 liters of deionized water to dissolve. After dissolution of ingredients in water and equilibration of solution temperature to room temperature, make up to 2.0 L mark with deionized water.
Porcine bladder with urethra, Animal Biotech Industries Inc., (Danboro, PA 18916)
Citrated bovine whole blood: Lampire Biological Laboratory Bovine CPD, Catalogue No. 7720010.
Cyanoacrylate A: Permabond 910 Tissue Adhesive, Catalogue No. 72590.
Cyanoacrylate B: Loctite 4902 instant adhesive Catalogue No. 1875841
Dialysis Tubing: 3,500 Da MWCO Snakeskin Dialysis Tubing (Fisher Scientific), Catalogue No. PI88244.
Parafilm: “M” Laboratory film, Pechiney plastic packaging (Chicago, IL 60631)
FeCl₃: Sigma Aldrich anhydrous grade Catalogue No. 8039450500 500 g
Gelatin: Sigma, Porcine Gelatin Bloom 300 Type A Catalogue No. G2500
FLOSEAL Hemostatic Matrix 5 ml, Baxter Product No. ADS201844, Lot No. (10) HA220132, EXP Nov. 29, 2023
HEMOSPRAY Powder, Cook Medical, PN No. G56572, Lot No. W4337018, EXP Apr. 2, 2023
HEMOSPRAY Powder, Cook Medical, PN No. G56572, Lot No. W4529951, EXP Oct. 26, 2024
Quartz UV test cells, 1 cm path length: HACH Co., Catalogue No. 48228-00

### Example 1

### Preparation of Catechol Chitosan and Characterization

Described herein, in chemical formulation and preparation, are Approaches 1 and 2 of preferred crosslinked chitosan gelatin materials (used as additives), and Approaches 3 to 8 of catechol modified chitosan materials synthesized by a preferred N-acylation reaction between a catechol molecule containing a terminal carboxylic acid and the C-2 amine of glucosamine mer of the chitosan.

Although not presented here in example form, this disclosure also includes a preferred catechol modified chitosan synthesis being an N-acylation of the C-2 amine on the chitosan glucosamine and the amine on 3,4-Dihydroxyphenethylamine (alternatively named dopamine) with carboxylic acid groups on a tricarboxylic acid including citric, isocitric and aconitic acid.

Although not presented here in example form, this disclosure also includes a preferred catechol modified chitosan synthesis being a reductive alkylation of the C-2 amine on the chitosan glucosamine by addition of 3,4-dihydroxybenzaldehyde to the chitosan with reduction of the intermediate imminium functional group using sodium cyanoborohydride or sodium borohydride.

The N-acylation catechol modified chitosan Examples are provided below in Approaches 3 to 8. Following the covalent chemical attachment (modification) of the catechol at the chitosan C-2 amine, the degree of substitution of the chitosan for approaches 3 to 8 was determined as follows: Quartz UV test cells, 1 cm path length, ×2 were used in acquiring UV/vis spectra at room temperature. The UV/Vis spectrophotometer was a Varian Cary Bio 100. Standard solutions of 3,4-dihydroxyhydrocinnamic acid were prepared in water and absorbance at 280 nm was plotted against concentration. The extinction coefficient (ε) was determined to provide for quantitative use in the Beer Lambert relationship (shown below) for absorbance in dilute solution.

$$A = ε \cdot c \cdot l$$

A is absorbance (dimensionless) and l/cm is the path length and ε/L·mol⁻¹·cm⁻¹ is the extinction coefficient.

The extinction coefficient (absorbance<0.5) of the aromatic catechol at 280 nm peak absorbance was determined as 2,540±50 liter/(mol·cm). This value was used to determine degree of substitution in the modified chitosan in dilute aqueous solution of known mass of modified chitosan, known volume of solution and measured peak absorbance at 280 nm.

The chitosan catechol solution was diluted so that its absorbance at 280 nm was less than 0.5 (usually about 1:50 or 1:100). The absorbance, the weight of the solution used in the dilution, and the percent solids (CS-catechol) were used to find the fractional degree of substitution (f_{DS}) of the HCA with respect to free amines on the chitosan backbone according to the equations:

$${f}_{D S} = \frac{{n}_{HCA}}{{f}_{DDA} \cdot {n}_{total Chitosan mers}}$$

$${f}_{D S} = \frac{A \cdot V \cdot \{ ( {f}_{D D a} \cdot 161 ) + ( 1 - {f}_{DDA} \cdot 203 ) \}}{ε \cdot l \cdot \{ {m}_{c c} - ( \frac{A \cdot V}{ε \cdot l} \cdot 165. 17 ) \} \cdot {f}_{DDA}}$$

where A is UV/vis absorbance at 280 nm of the modified chitosan; V is the volume (liters) of the modified chitosan solution taken to dry to constant dry mass; mcc is the measured dry mass (g) of the catechol modified chitosan; f_{DDA} is the fractional degree of deacetylation of the chitosan.

Examples of two crosslinked chitosan gelatin materials (no catechol modification) and five catechol modified chitosan materials are provided in the eight Approaches provided below.

### Approach 1

### Chitosan Gelatin Crosslinked Matrix 1:4 Chitosan to Gelatin (CsGelatin1):

Prepare a 25% gelatin solution (Sigma, Porcine gelatin Bloom 300 Type A) in DI water. For example, add 16 g gelatin to 48 mL DI water, stir to dissolve.

Prepare a 2% chitosan solution (chitosan 43000 Primex) in 2% acetic acid (Glacial) where the mass of chitosan is four times less than the mass of gelatin (1:4 chitosan to gelatin final ratio). The percent solids of chitosan are accounted for. For example, add 4.4 grams of chitosan to 191 mL DI water and 3.8 mL acetic acid. Stir to dissolve.

Slowly add the chitosan solution to the gelatin solution while stirring.

Heat the solution at 40-50° C. for 0.5-2 hours, stirring occasionally.

Add 1-Ethyl-3-(3-dimethylaminopropyl) carbodiimide (EDC) to a final concentration of 30 mM while stirring.

Spread the gel into 150 mm Petri dishes at 75-100 g per aliquot each and leave out to dry until hardened.

Alternatively, spread the gel out into petri dishes at 50-75 g per dish and lyophilize for 48 hours.

Once the gel is dried, cryogrind to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6.0-5.0 Hz. After milling, the powder is sieved to remove particles with radius of gyration<12.5 micron. The final dry powder (% moisture≤15% w/w) is stored in closed container in a cool, dry location.

### Approach 2

### Chitosan Gelatin Crosslinked Matrix 1:24 Chitosan to Gelatin (CsGelatin2)

Prepare a 12% gelatin solution (Sigma, Porcine gelatin Bloom 300 Type A) in DI water. For example, add 24 g gelatin to 176 mL DI water, stir to dissolve.

Prepare a 2% chitosan solution (chitosan 43000 PRIMEX) in 2% Acetic Acid (Glacial) where the mass of chitosan is twenty-four times less than the mass of gelatin (1:24 chitosan to gelatin final ratio) and the chitosan solution is four time less by volume (1:4 chitosan solution to gelatin solution by volume). The percent solids of chitosan are accounted for. For example, add 1.11 grams of chitosan to 50 mL DI water and 0.95 mL acetic acid. Stir to dissolve.

Slowly add the chitosan solution to the gelatin solution while stirring.

Heat the solution at 40-50° C. for 0.5-2 hours, stirring occasionally.

Add 1-Ethyl-3-(3-dimethylaminopropyl) carbodiimide (EDC) to a final concentration of 30 mM while stirring.

Spread the gel into 150 mm Petri dishes at 75-100 g per aliquot each and leave out to dry until hardened.

Once the gel is dried, cryogrind to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6 Hz. After milling, the powder is sieved to remove particles with radius of gyration<12.5 micron. The final dry powder (% moisture≤15% w/w) is stored in closed container in a cool, dry location.

### Approach 3

### Type Z Catechol-Modified Chitosan

In one embodiment, the method comprises: preparing a 1% w/w aqueous chitosan solution at pH 5.5, adding Fe(III) salt (at molar equivalence to catechol in the following step), then performing a synthesis with the chitosan, iron, and catechol in an aqueous reaction solution to bring the chitosan solution to 0.5% w/w. The reaction solution pH is maintained between 5.3-5.7 with target pH 5.5, then increased while dialyzed against DI water adjusted to a target pH 6.0-6.3 to provide catechol oxidation and crosslinking. The fractional degree of substitution (fps) of the Type Z catechol modified chitosan is in the range 0.50-0.90.

The Type Z powder is prepared as follows. The method of solidification of Type Z catechol modified chitosan solid membrane sheet is by freeze phase separation of the catechol modified chitosan solution near −40° C. with subsequent sublimation removal of the water to moisture content to less than 5% w/w of the final freeze-dried membrane. The final freeze-dried membrane is milled at its original density near 0.005 g/cm³. The final membrane is cryoground to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6.0-5.0 Hz. After milling, the powder is sieved to remove particles with radius of gyration<12.5 micron. The final dry powder (% moisture≤15% w/w) is stored in closed container in a cool, dry location.

### Approach 4

### Type Y Catechol-Modified Chitosan

In one embodiment, the method comprises preparing a 1% w/w aqueous chitosan solution at pH 5.5 and performing a synthesis with the chitosan and catechol in an aqueous reaction solution to bring the chitosan solution to 0.5% w/w. An iron salt is then added, at five time less molar quantity than that of the catechol. The reaction solution pH is maintained between 5.3-5.7 with target pH 5.5, then increased while dialyzed against DI water adjusted to target pH 6.0-6.3 to provide catechol oxidation and crosslinking. The fractional degree of substitution (f_{DS}) of the Type Y catechol modified chitosan is in the range 0.30-0.70.

The Type Y powder is prepared as follows. In a preferred embodiment, the method of solidification of Type Y catechol modified chitosan solid membrane sheet from solution is by freeze phase separation of the catechol modified chitosan solution at close to −40° C. with subsequent sublimation removal of the water to moisture content to less than 5% w/w of the final freeze-dried membrane. The final freeze-dried membrane is milled at its original density near 0.005 g/cm³. The final membrane is cryoground to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6.0-5.0 Hz. After milling, the powder is sieved to remove particles with radius of gyration<12.5 and >125 microns. The final dry powder (% moisture≤15% w/w) is stored in closed container in a cool, dry location.

### Approach 5

### Type Y1 Catechol-Modified Chitosan

In one embodiment, the method comprises preparing a 1% w/w aqueous chitosan solution at pH 5.5 and performing a synthesis with the chitosan and catechol in an aqueous reaction solution to bring the chitosan solution to 0.5% w/w. An iron salt is then added, at five time less molar quantity than that of the catechol. The reaction solution pH is maintained between 5.3-5.7 with target pH 5.5, then increased while dialyzed against DI water adjusted to a higher pH of target 6.0-6.3 to provide catechol oxidation and crosslinking. The fractional degree of substitution (fps) of the Type Y catechol modified chitosan is in the range 0.30-0.70.

The Type Y1 powder is prepared as follows. In a preferred embodiment, the method of solidification of Type Y catechol modified chitosan solid membrane sheet from solution is by freeze phase separation of the catechol modified chitosan solution at close to −40° C. with subsequent sublimation removal of the water to moisture content to less than 5% w/w of the final freeze-dried membrane. The final freeze-dried membrane is milled at its original density near 0.005 g/cm³. The final membrane is cryoground to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6.0-5.0 Hz. After milling, the powder is sieved to remove particles with radius of gyration<31.5 and >125 microns. The final dry powder (% moisture≤15% w/w) is stored in closed container in a cool, dry location.

### Approach 6

### Type Y2 Catechol-Modified Chitosan

In one embodiment, the method comprises preparing a 2% w/w aqueous chitosan solution at pH 5.5 and performing a synthesis with the chitosan and catechol in an aqueous reaction solution to bring the chitosan solution to 1% w/w. An iron salt is then added, at five time less molar quantity than that of the catechol. The reaction solution pH is maintained between 5.3-5.7 with target 5.5, then increased in dialysis (×4) against DI water adjusted to higher pH of target 6.0-6.3 to provide catechol oxidation and crosslinking. The fractional degree of substitution (f_{DS}) of the Type Y2 catechol modified chitosan was determined to be is in the range 0.30-0.70.

The Type Y2 powder is prepared as follows. In a preferred embodiment, the method of solidification of Type Y2 catechol modified chitosan solid membrane sheet from solution is by freeze phase separation of the catechol modified chitosan solution at close to −40° C. with subsequent sublimation removal of the water to moisture content to less than 5% w/w of the final freeze-dried membrane. The final freeze-dried membrane is milled at its original density near 0.01 g/cm³. The final membrane is cryoground to a powder using a cryogenic hammer mill such as a universal micropulverizer hammer mill. The membrane is cut into smaller pieces before milling. The membrane material and its container are pre-cooled for close to 20 minutes under liquid nitrogen. The micropulverizer mill is run above 1200 rpm under liquid nitrogen with an extraction sieve in place. After milling, the powder is further sieved to remove particles with radius of gyration<12.5 and >125 microns. The final dry powder (% moisture≤15% w/w) is stored in closed container in a cool, dry location.

### Approach 7

### Type Y3 Catechol-Modified Chitosan

In one embodiment, the method comprises preparing a 2% w/w aqueous chitosan solution at pH 5.5 and performing a synthesis with the chitosan and catechol in an aqueous reaction solution to bring the chitosan solution to 1% w/w. An iron salt is then added, at five time less molar quantity than that of the catechol. The reaction solution pH is maintained between 5.3-5.7 with target 5.5, then increased in dialysis (×4) against DI water adjusted to higher pH of target 6.0-6.3 to provide catechol oxidation and crosslinking. The fractional degree of substitution (fps) of the Type Y2 catechol modified chitosan was determined to be is in the range 0.30-0.70.

The Type Y3 powder is prepared as follows. In a preferred embodiment, the method of solidification of Type Y3 catechol modified chitosan solid membrane sheet from solution is by freeze phase separation of the catechol modified chitosan solution at close to −40° C. with subsequent sublimation removal of the water to moisture content to less than 5% w/w of the final freeze-dried membrane. The final freeze-dried membrane is milled at its original density near 0.01 g/cm³. The final membrane is cryoground to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6.0-5.0 Hz. After milling, the powder is sieved to remove particles with radius of gyration<31.5 and >125 microns. The final dry powder (% moisture≤15% w/w) is stored in closed container in a cool, dry location.

### Approach 8

Catechol-Modified Chitosan with Chitosan Acetate Backing

In one embodiment, the method comprises preparing a first aqueous solution of 0.5% w/w catechol modified chitosan according to approaches 3 to 5 (Type Z, Y or Y2). A second aqueous solution of 0.5% w/w chitosan acetate is prepared by dissolution of chitosan powder in an aqueous solution of acetic acid by maintaining solution pH below 4 and stirring.

A catechol modified chitosan with chitosan acetate powder combination is prepared as follows. In a preferred embodiment, the method of solidification of catechol modified chitosan and chitosan acetate solid membrane sheet is by two layer freeze phase separation. A clear, flat-based polystyrene 150 mm diameter petri dish with 15 mm wall height is used to contain the combined solutions. The catechol modified solution is poured first to fill the mold to a height of close to 4 mm. The mold and its solution are then placed on a freezing plate at close to −40° C. to freeze the aqueous solution. The 0.5% w/w solution of chitosan acetate is then poured to add a further 2 mm height of solution over the top surface of the frozen catechol modified chitosan. The mold and its contents are then placed on a heat transfer shelf near −40° C. in the freeze dryer to freeze both layers with induced freeze phase separation of the solute components. A freeze-drying cycle is completed to achieve sublimation removal of the water to a final moisture content of <5% w/w. The final freeze-dried membrane comprising a base layer of catechol modified chitosan and a top layer of chitosan acetate with weight ratio of catechol modified chitosan to chitosan acetate near 2:1 is milled at its original density near 0.005 g/cm³. The final membrane is cryoground to a powder using a cryogenic hammer mill such as a SPEX 6775 mill. The membrane is cut into smaller pieces before milling. The membrane material and its container are pre-cooled for close to 15 minutes under liquid nitrogen. The SPEX mill run time is close to 2.0-1.5 minutes per cycle, with cool time of 1 min, 4 cycles and hammer cycle rate of 6.0-5.0 Hz. After milling, the powder is sieved to remove particles with radius of gyration<12.5 micron. The final dry powder (% moisture≤15% w/w) is stored in closed container in a cool, dry location.

### Example 2

### Characterization of Cryoground Powders

The cryoground powders of Approaches 1, 3 & 4 of Example 1 were characterized for their chemical composition by mid-field (4000-600 cm⁻¹) fourier transform infrared (FTIR) attenuated total reflectance (ATR) spectroscopy. Powder density was characterized by determination of Bulk Density, Tapped Density, and Compressibility. Density determinations were performed manually without specialized equipment on individual powder components, not combined final powders to be mixed in for the flowable hemostat.

### Fourier-Transform Infrared Spectroscopy

Representative Fourier-transform Infrared (FTIR) absorbance spectra between 4000 to 600 cm⁻¹ of the powders of Approaches 1, 3 & 4 of Example 1 were obtained using 32 scans at 4 cm⁻¹ resolution with the ZnSe ATR accessory of a ThermoNicolet Avatar 380 FTIR spectrophotometer (FIGS. 1-3 respectively).

### Powder Bulk Density

The bulk density of a powder is the ratio of the mass of an untapped powder sample and its volume including the contribution of the interparticulate void volume” (Bulk Density and Tapped Density of Powders)

### Procedure:

The powder is passed through a sieve with apertures greater than or equal to 1.0 mm. As much powder as needed is weighed to complete the test (will depend on how much powder is available. Target weight is 5 g. The powder is added into a graduated cylinder that can be read to within 1 to 2 ml.

The unsettled volume of the powder is recorded with bulk density calculated in g/ml

This was repeated ×3.

### Powder Tapped Density

Tapped density is the increased bulk density attained after mechanically tapping a container containing the powder sample.

### Procedure:

Tapped density was performed manually as follows. The graduated cylinder containing powder as prepared in the bulk density determination with volume and mass recorded was elevated 3±0.2 mm and allowed to fall under its own weight. This was performed for ten taps with volume recorded at completion of the tenth tap. An additional ten taps for a total of 20 taps was performed with the 20th tap volume recorded. Multiple sets of 10 taps were performed until the difference between measurements was less than or equal to 2 mL. Once this is achieved, a final tap density (g/mL) and volume change is recorded. This process was repeated three times for each powder type.

### Powder Compressibility

Comparison of bulk and tapped densities provides a measure of the interparticle interactions of a powder. These interactions affect the ability of a powder to flow and are expressed using the compressibility index or the Hausner ratio. The compressibility index and Hausner ratio reflect the ability of a powder to be compressed. In a powder that can flow freely, these interactions are less significant, and the bulk and tapped densities will be closer in value. In poorly flowing powders, there are frequently greater interparticulate interactions, and a greater difference between the bulk and tapped densities will be observed. These differences are reflected in the Compressibility Index and the Hausner Ratio.

$$Compressibility Index = \frac{100 ( {V}_{0} - {V}_{f} )}{{V}_{0}}$$

$$where {V}_{0} = unsettled apparent volume$$

$${V}_{f} = final tapped volume$$

$$Hausner Ratio = \frac{{V}_{0}}{{V}_{f}}$$

### FTIR Spectra

Please see FIG. 1 for FTIR spectrum of CsGelatin1 dried material.

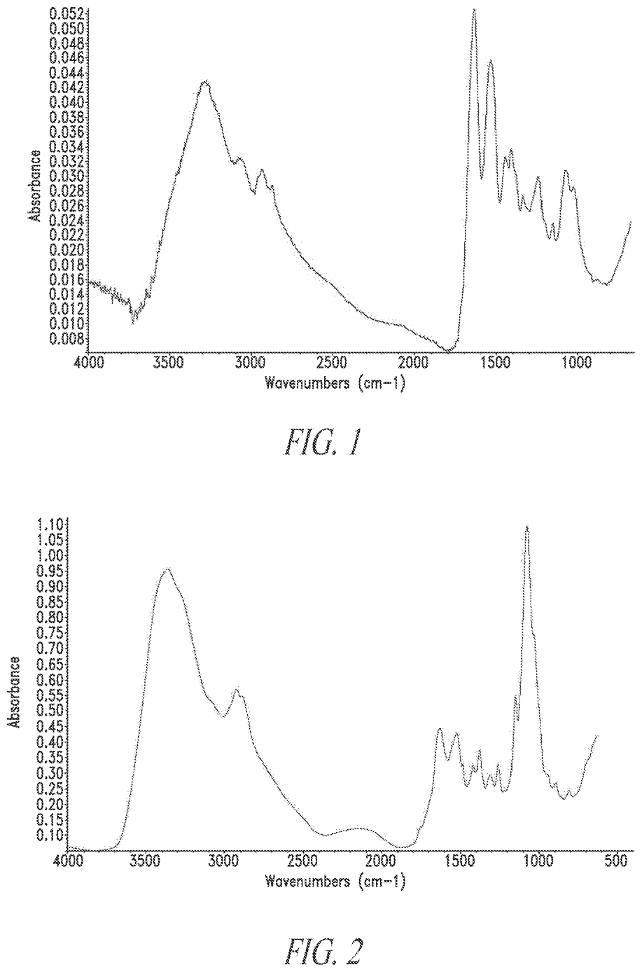

Please see FIG. 2 for FTIR spectrum of Type Z freeze dried material.

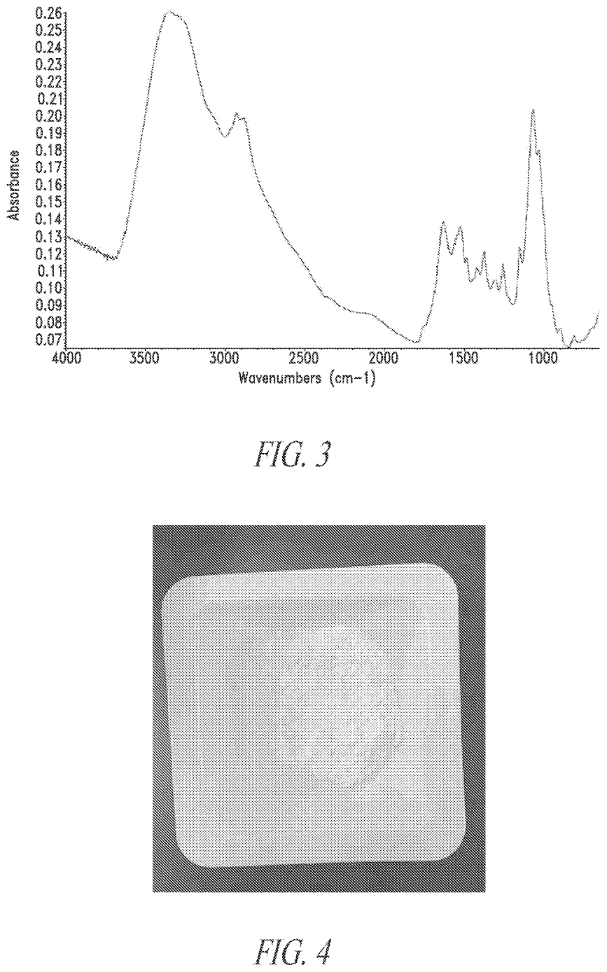

Please see FIG. 3 for FTIR spectrum of Type Y freeze dried material.

### Bulk and Tapped Densities of Powders

TABLE 1
Average Bulk and Tapped Densities of Cryoground Powders

| | Average Bulk Density | Average Tapped Density |
|---|---|---|
| Description of Powder | (g/mL) | (g/mL) |
| CsGelatin1 Powder | 0.607 ± 0.006 | 0.805 ± 0.019 |
| CsGelatin2 Powder | 0.548 ± 0.006 | 0.803 ± 0.002 |
| Type Z Powder | 0.365 ± 0.002 | 0.566 ± 0.026 |
| Type Y Powder | 0.166 ± 0.01 | 0.331 ± 0.02 |

### Powder Compressibility

TABLE 2
Average Compressibility Index and Hausner Ratio of Powders

| | Average Compressibility | |
|---|---|---|
| Lot | Index | Average Hausner Ratio |
| CsGelatin1 Powder | 24.5 ± 2.1 | 1.3 ± 0.0 |
| CsGelatin2 Powder | 31.7 ± 0.6 | 1.4 ± 0.0 |
| Type Z Powder | 35.4 ± 3.1 | 1.6 ± 0.0 |
| Type Y Powder | 50.0 ± 2.8 | 2.0 ± 0.1 |

### Example 3

### Preparation of Flowable Chitosan Dressing from Powder and Water

The selected mass of powder is weighed and added to a first syringe with a mating end connector such as a screw type or Luer end connector that is capped. If filling and mixing two types of powder into the syringe such as Types Z and Cs-Gelatin, the selected amounts (typically 1:1 by mass) are weighed into the syringe one after the other. On loading the powder, the syringe piston (plunger) is placed partially inside the powder filled barrel of the first syringe, sealing the large opening end of the syringe, and in the process care is taken not to lose any powder. The syringe is inverted to allow the powder to fall onto syringe piston end. In the case of loading of more than one powder type, the powders can be mixed by shaking agitation inside the syringe until the mixture appears homogeneous. Keeping the syringe inverted with powder resting under gravity against the plunger, the capped end of the syringe is opened to allow slow depression of the plunger to expel air from the syringe without loss of any powder or substantially compaction of the powder. After expelling the air, the cap is reattached to the syringe end to secure the first syringe contents.

In a second syringe with opposite mating connector to the first syringe and similar volume to the first syringe, a chosen volume of water is drawn into the syringe while avoiding the drawing of air. After drawing the chosen volume, the syringe end is capped. A typical volume of water is 6 to 8 times the volume (ml) per mass (g) of dry powder for Type Z, Type Y and Type Z/Cs-Gelatin dressings. Thus for 0.5 g of powder mixture, typically 3.5 ml of water is added to the second syringe. A typical volume of water is 15 to 20 times the volume (ml) per mass (g) of dry powder for Type Y2 dressings. Thus for 0.5 g of powder mixture, typically 8.5 ml of water is added to the second syringe. A preferred syringe volume is 2.5 to 3.5 times the volume of liquid added to the second syringe. The syringes are inspected to ensure both syringe pistons are secure with both syringe ends securely capped.

The twin syringes may be packaged with their delivery catheter as a medical device in single closure packaging (both syringes together) or packaged separately in their own closed portions of the device package prior to terminal sterilization at sterilization assurance level (SAL) 10⁻⁶ by high energy irradiation such as with electron beam, x-ray or gamma irradiation. The flowable dressing may be stored for a prolonged period at between 25° C. to 4° C. before use.

At point of care, the syringes are removed from their packaging with removal of the syringe end caps. The mating ends of the syringes are joined providing for closed connection of the two syringes. The piston of the second syringe with the liquid medium is pressed down causing the liquid to fill and mix with the powder of the first syringe and the piston of the first syringe to press up. After full depression of the piston of the second syringe, the raised piston of the first syringe is fully depressed to express the full partially mixed volume of the first syringe back into the second syringe. The pistons of both syringes are pressed back and forth at about 1 full depression a second to mix the liquid and solid powder components of the flowable dressing. After about 30 seconds, a thick and consistent fluid dispersion is formed. The desirable fluid properties of the flowable dressing make it suitable for delivery and use for at least 15 minutes to 1 hour after mixing.

The final syringe used to connect to the delivery conduit (a tube with a matching end connector) is the syringe that is filled with the flowable mixture following the final mixing piston depression. The final syringe is then connected to the proximal end of a single lumen delivery conduit or a catheter. The flowable dressing is delivered by the delivery device compression (for example depression of the syringe piston) through the delivery conduit or catheter distal end onto the wound site or affected area. If the flowable dressing composition is unable to be fully expelled from the delivery catheter volume then a liquid including by not limited to sterile water and 0.9% w/w aqueous saline solution may be added to the syringe connecting end (proximal end) of the delivery catheter to fully expel the flowable dressing material from the catheter distal end. An alternate method to expel the remaining flowable dressing from the catheter is to pressurize the syringe connecting end of the delivery catheter with a gas including but not limited to atmospheric air, nitrogen, and argon. A small, movable, solid plug with diameter that is close to the internal diameter of the catheter and plug length greater than diameter may be used inside the catheter immediately against the proximal end of the flowable dressing to avoid interfacial mixing of the flowable dressing with the liquid or gas used to expel the remaining flowable dressing from the catheter. Preferably the plug material is formed of an insoluble chitosan material. In a preferred embodiment, the flowable dressing is applied from the distal end of the catheter onto the mucosal wound site in layering, painting manner. In a preferred embodiment, the flowable dressing may be applied from the catheter distal end to fill a bleeding cavity. As a thick layer (2-5 mm in thickness) is applied over bleeding areas, blood infiltrates through the catechol modified chitosan matrix initiating a firm adherent clot that quickly stops bleeding in all patients including those on anticoagulation and antiplatelet therapy. Irrigation of the treated area may be used within 5-10 minutes of bleeding stopping to investigate the robustness of the hemostasis and if required, to allow accurate placement of further flowable dressing on locations demonstrating need for further application.

Table 3 provides recommended ratios of water to powder for different catheter sizes.

TABLE 3
Powder to Water Ratios for Chitosan
Gelatin Type Z Flowable Dressing

| Working | | |
|---|---|---|
| Channel | | |
| Size (mm) | Powder Components | Water Component |
| 2.8 | Type Z: Cs-Gelatin; Type | 7x the mass of powder |
| | Z; Type Y | |
| 3.7 | Type Z: Cs-Gelatin; Type | 6-6.5x the mass of powder |
| | Z; Type Y | |

### Example 4

### Images of Flowable Chitosan Hemostat Powders, Mixing and Delivery

Please see FIG. 4 for an image of CsGelatin1 powder.

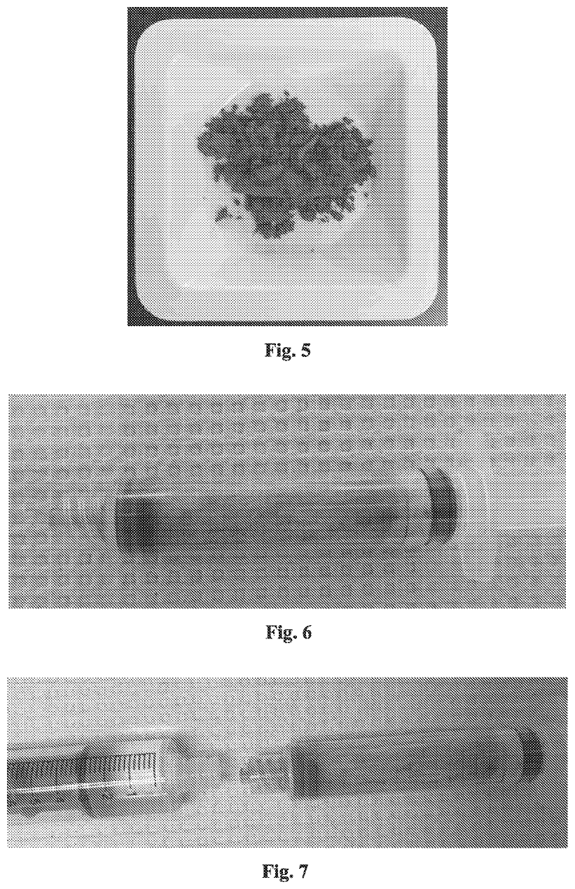

Please see FIG. 5 for an image of Type Z catechol modified powder.

Please see FIG. 6 for an image of 1st syringe with male Luer screw connector end of example 3 with powder loaded in syringe (no cap).

Please see FIG. 7 for an image of 1st syringe and 2nd syringe with Female Luer connector (on left with liquid) of example 3 prior to syringes connected to each other.

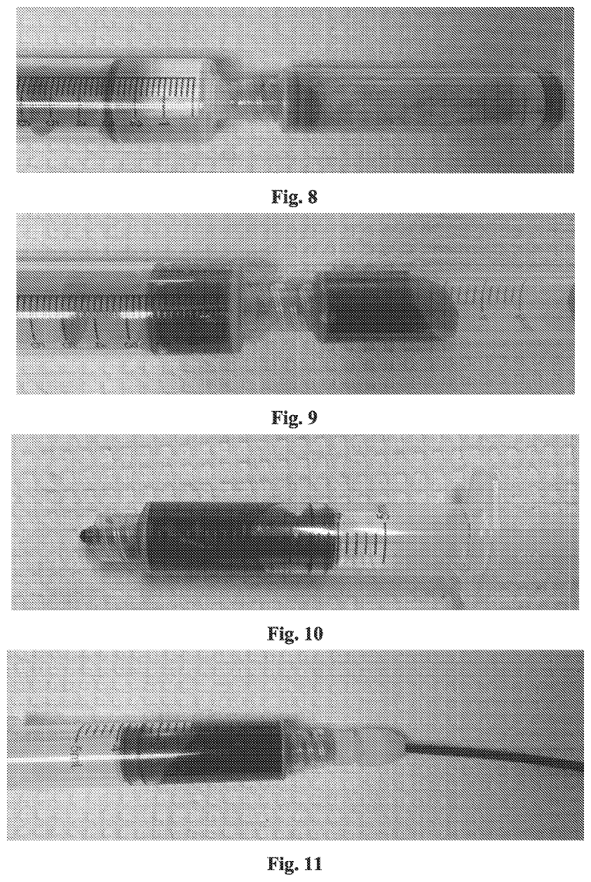

Please see FIG. 8 for an image of 1st syringe and 2nd syringe (on left with liquid) of example 3 with syringes connected to each other by female (on left) and male (on right) screw connector ends.

Please see FIG. 9 for an image of 1st syringe and 2nd syringe of example 3 with syringes connected to each other by male and female screw connector ends and with flowable particles substantially dispersed (mixed) uniformly into the liquid without foaming or other signs of gas bubbles.

Please see FIG. 10 for an image of 1st syringe with male Luer connector containing uniformly dispersed particle-liquid mixture ready to be delivered.

Please see FIG. 11 for an image of 1st syringe containing uniformly dispersed particle-liquid mixture ready to be delivered and connected to a catheter delivery tube for accurate, localized minimally invasive application to an injury.

Please see FIG. 12 for an image of deployment of flowable dressing bead from 1st syringe delivery catheter end onto a horizontal, transparent PVC plate with central 4.0 mm diameter hole.

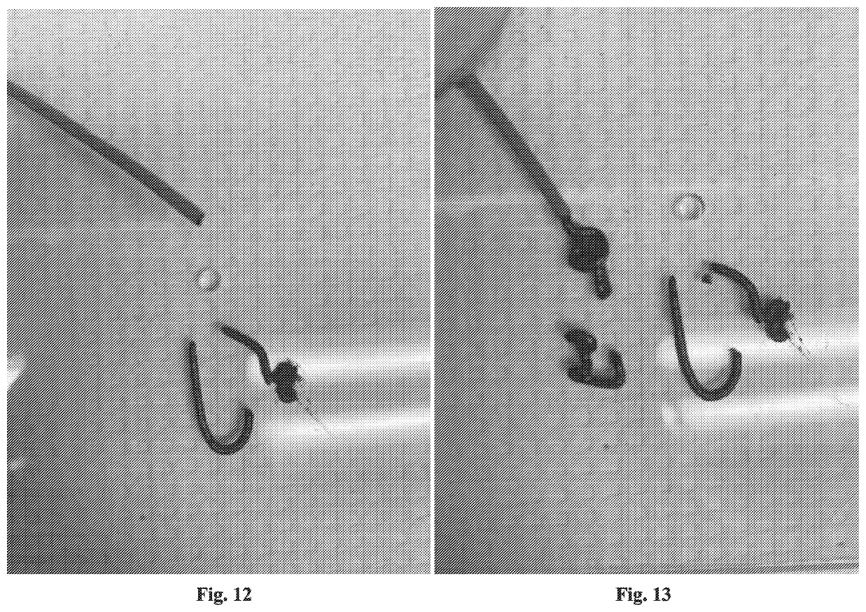

Please see FIG. 13 for an image of deployment of flowable dressing bead from 1st syringe delivery catheter end onto a horizontal, transparent PVC plate with central 4.0 mm diameter hole.

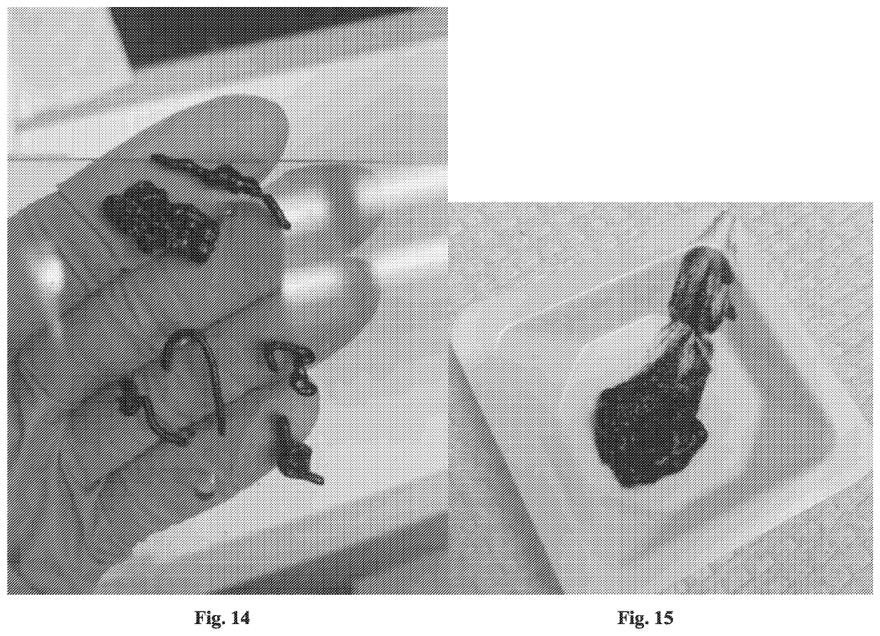

Please see FIG. 14 for an image of deployment of adhered flowable dressing bead forms from 1st syringe delivery catheter end onto an upright, clear PVC plate with central 4.0 mm diameter hole.

Please see FIG. 15 for an image of all flowable dressing (close to 3.5 ml) from 1 st syringe delivery catheter transferred to partially cover base of thermoformed polystyrene dish (10 cm×10 cm).

### Example 5

### Ex-Vivo Testing of Type Z & Cs-Gelatin Flowable Chitosan Dressings

Flowable hemostatic dressing compositions of the invention were tested against a FLOSEAL control for their ability on delivery to adhere, and remain adhered, to different types of ex-vivo freshly harvested porcine tissue. The flowable hemostat of the invention was first tested for cohesion and tissue adhesion at sites of delivery to ex-vivo tissue to investigate ability to stay adhered and resist effects of gravity (e.g. upside down) without flowing off the application site. The flowable hemostatic dressing compositions were also investigated for ability to remain adhered to different tissue types in difficult wet environments (e.g. in urine in the bladder; in gastrointestinal fluid; in a congested airway or nasal passage or other) without significant loss of adhesion and cohesion properties over an extended time. The importance of identifying catechol modified chitosan compositions that dependably adhere and stay adhered to these key tissue types is made apparent by the fact that there are no known hemostatic compositions for treatment of local bleeding in the bladder and its urological tracts; and currently available hemostatic agents for use in the gastrointestinal tract are unable to provide anything more than acute bleeding control of difficult bleeding in the stomach without demonstrated reduction in incidence of rebleeding beyond 12 hours. The ex-vivo beaker test allows investigation of persistence and extent of test samples remaining attached over different tissue types under wet conditions that mimic in vivo wet conditions.

Flowable hemostatic compositions of i) Type Z, ii) 1:1 Type Z and CsGelatin2; iii) CsGelatin2 alone were investigated in benchtop studies. The freshly harvested porcine tissues were: 1) stomach mucosal tissue, 2) liver, 3) esophagus, and 3) bladder. The testing was performed at 37° C. fully submerged in wet environments which were: 1) synthetic gastric fluid was used for the stomach tissue, 2) synthetic urine for the bladder tissue, and 3) 0.9% isotonic saline solution for the liver and esophagus.

FLOSEAL Hemostatic Matrix (Baxter) flowable dressing (5 ml) was used as a control flowable dressing under the same test conditions used for the chitosan flowable dressings of the invention.

### Definitions

| Term | Definition |
|---|---|
| CEHFD | Chitosan Endoluminal hemostatic flowable dressing |
| CGHFD | Chitosan Gastrointestinal hemostatic flowable dressing |
| GI | Gastrointestinal |
| TURP | Transurethral resection of the prostate |
| Type Z | Catechol modified iron enhanced chitosan material (f_{DS} = |
| | 0.5-0.9) |
| Type Y | Catechol modified iron enhanced chitosan material (f_{DS} = |
| | 0.3-0.7) |
| Cs-Gelatin | Chitosan gelatin crosslinked material (includes CsGelatin1 |
| | and CsGelatin2 examples) |
| CsGelatin1 | Chitosan gelatin crosslinked material at 1:4 w/w |
| CsGelatin2 | Chitosan gelatin crosslinked material at 1:24 w/w |
| 1 to 1 | 1:1 w/w Type Z and CsGelatin2 |
| FLOSEAL | FLOSEAL Hemostatic Matrix, Baxter |

TABLE 4
Type Z and CsGelatin2 Masses Used in Flowable
Compositions for Ex-vivo Testing

| | | | Mass | |
|---|---|---|---|---|
| | Label and | Mass Type | CsGelatin2 | Total |
| Formulation | Test | Z (g) | (g) | Mass (g) |
| Type Z Only | A-GI | 0.207 | 0 | 0.207 |
| 1 to 1 | B-GI | 0.113 | 0.118 | 0.231 |
| CsGelatin2 alone | C-GI | 0 | 0.327 | 0.327 |
| Type Z alone | D-Liver | 0.209 | 0 | 0.209 |
| 1 to 1 | E-Liver | 0.108 | 0.111 | 0.219 |
| CsGelatin2 alone | F-Liver | 0 | 0.428 | 0.428 |
| Type Z alone | A-TURP | 0.2973 | 0 | 0.2973 |
| 1 to 1 | B-TURP | 0.1232 | 0.1237 | 0.2469 |
| CsGelatin2 alone | C-TURP | 0 | 0.2482 | 0.2482 |
| Type Z alone | D-Esophagus | 0.2478 | 0 | 0.2478 |
| 1 to 1 | E-Esophagus | 0.1256 | 0.1244 | 0.2500 |
| CsGelatin2 alone | F-Esophagus | 0 | 0.2490 | 0.2490 |

TABLE 5
Water Volume Used in Flowable Compositions for Ex-vivo Testing

| | | | Target | H₂O |
|---|---|---|---|---|
| | Label | Powder:Water | H₂O | Added |
| Formulation | and Test | Ratio | (ml) | (ml) |
| Type Z alone | A-GI | 1:3.5 | 0.72-0.8 | 0.75 |
| 1 to 1 | B-GI | 1:6 | 1.38 | 1.3 |
| CsGelatin2 alone | C-GI | 1:4.5 | 1.47 | 1.5 |
| Type Z alone | D-Liver | 1:3.5 | 0.73-0.8 | 0.75 |
| 1 to 1 | E-Liver | 1:6 | 1.31 | 1.3 |
| CsGelatin2 alone | F-Liver | 1:4.5 | 1.9 | 1.9 |
| Type Z alone | A-TURP | 1:3.5 | 0.8-0.87 | 0.8 |
| 1 to 1 | B-TURP | 1:6 | 1.41-1.9 | 1.4 |
| CsGelatin2 alone | C-TURP | 1:4.5 | 1.05-1.12 | 1.0 |
| Type Z alone | D-Esophagus | 1:3.5 | 0.8-0.87 | 0.75 |
| 1 to 1 | E-Esophagus | 1:6 | 1.45-1.51 | 1.4 |
| CsGelatin2 alonel | F-Esophagus | 1:4.5 | 1.08-1.13 | 1.0 |

### Methods

TABLE 6
Preparation of Tissues

| Tissue | Preparation |
|---|---|
| Stomach | Lining removed; mucosa cut into ~1″ × 1″ pieces |
| | (no simulated wound crater). |
| Bladder | Lining removed; cut into ~1″ × 1″ pieces (no simulated |
| | wound crater). |
| Liver | ~1″ × 1″ pieces of hepatic lobe (simulated wound with |
| | 0.6″ diameter punch and surgical scissors used to |
| | create ~0.25″ deep defect/crater). |
| Esophagus | Esophagus cut down center and splayed, cut |
| | into ~1 × 2″ pieces (no simulated wound crater). |

The base of the tissue was fixed with cyanoacrylate cement to the bottom of a 150 mL polystyrene beaker cup. The top surface of the adhered tissue was wet with one to two drops of test system solution (synthetic urine, synthetic gastrointestinal fluid or 0.9% saline).

The chitosan flowable dressings were prepared as follows: One male Luer 5 mL syringe contained the weighed, dry powder component. A corresponding female luer lock syringe was filled with the volume of water specified in Table 5. The syringes were connected and mixed for 30 seconds. The male Luer syringe with the flowable mixture was used to deliver the flowable dressing onto the tissue of a beaker test replicate pair.

The FLOSEAL was prepared according to the manufacturer's directions. For each test tissue preparation, 3-4 drops of bovine blood were applied centrally to the top surface of the tissue. The chitosan flowable hemostatic dressings were applied to their tissue surfaces using one syringe of chitosan flowable dressing per replicate pair, each receiving equal portions. For the FLOSEAL Samples, the 5 mL volume was equally distributed across four samples (around 1.2 ml per cup).

The flowable dressings were then allowed to sit on their respective wetted tissues for 30 minutes, after which their test solution systems were added to the polystyrene beaker to fully submerge the flowable dressings in synthetic urine, synthetic gastrointestinal fluid or 0.9% saline.

The test beakers with their tissue and test flowable dressings were placed on shelves inside a 37° C. incubator and allowed to sit with periodic observation of tissue adhesion and resistance of the flowable composition to their fully immersed wet environment. Evaporation of water from the beakers was blocked by sealing closed the top of a test beaker with Parafilm.

The chitosan flowable test samples and FLOSEAL flowable controls were subsequently monitored for detachment and extent of detachment from the tissue surface. Often on detachment of the catechol modified chitosan material, a less-cohesive upper layer or “mound” of bulk sample would separate from the tissue leaving in place a more firmly adhered lower layer. In the catechol modified chitosan materials, this lower layer often appeared as a tough, strongly adhered and uniform protective surface layer. The beaker test investigated the persistence of the applied test and control materials on their respective tissues under the different wet immersion conditions.

The time of observation of mound detachment was recorded with time provided as the mean time between when the flowable dressing mound was found detached and the last time it was seen attached. The flowable samples were also monitored for the time of the adhered lower layer being observed attached to the tissue surface. When this layer was observed as absent or depleted, time of observation of absence/significant depletion of surface residue was recorded with time provided as the mean time between when adhered dressing surface layer was found to be substantially absent and the last time it was seen to be present.

The flowable dressings test and control samples were monitored also for dissolution. The chitosan gelatin flowable dressing often fully dissolved in the fluid. The Type Z or mixed chitosan flowable did not completely dissolve but tended to break-down into small particles. The dissolution time for the Type Z or mixed chitosan flowable is the time it is completely broken up and could pass or be excreted from the body. This time is recorded as the first time the flowable dressing is seen to be completely broken up or dissolved.

### Results

Results of the ex-vivo tissue adhesion testing are provided in Tables 7 to 9 below:

TABLE 7
Average Detachment Time (hours) of bulk
(mound) sample material from Tissue

| Formulation | Stomach | Liver | Bladder | Esophagus |
|---|---|---|---|---|
| Type Z | 3.03 ± 0.84 | 17.47 ± 3.60 | 16.26 ± 11.18 | 1.26 ± 0.0 |
| 1 to 1 | 2.18 ± 0.0 | 4.62 ± 1.22 | 3.12 ± 0.0 | 1.26 ± 0.0 |
| CsGelatin2 | 4.01 ± 1.83 | 0.87 ± 0.0 | 1.26 ± 0.0 | 1.26 ± 0.0 |
| FLOSEAL | 0.25 ± 0.0 | 3.83 ± 0.0 | 0.25 ± 0.0 | 0.496 ± 0.25 |

TABLE 8
Average Time (hours) for absence of adhered sample material on Tissue

| Formulation | Stomach | Liver | Bladder | Esophagus |
|---|---|---|---|---|
| Type Z | 13.87 ± 0.0 | 26.49 ± 6.58 | 48.75 ± 0.0 | 1.26 ± 0.0 |
| 1 to 1 | 8.63 ± 5.23 | 18.23 ± 14.83 | 12.21 ± 0.0 | 1.26 ± 0.0 |
| CsGelatin2 | 4.01 ± 1.83 | 0.87 ± 0.0 | 1.26 ± 0.0 | 1.26 ± 0.0 |
| FLOSEAL | 0.74 ± 0.0 | 13.2 ± 0.0 | 1.81 ± 1.07 | 0.50 ± 0.25 |

TABLE 9
Average sample material Dissolution Time in test liquid

| Formulation | Stomach | Liver | Bladder | Esophagus |
|---|---|---|---|---|
| Type Z | 19.92 ± 0.0 | 26.49 ± 6.58 | 48.75 | 24.00 |
| 1 to 1 | 19.92 ± 0.0 | 26.49 ± 6.58 | 24.00 | 24.00 |
| CsGelatin2 | 19.92 ± 0.0 | 9.96 ± 0.0 | 6.17 | 6.17 |
| FLOSEAL | 1.23 ± 0.0 | 21.17 ± 0.0 | 25.37 ± 0.0 | 25.37 ± 0.0 |

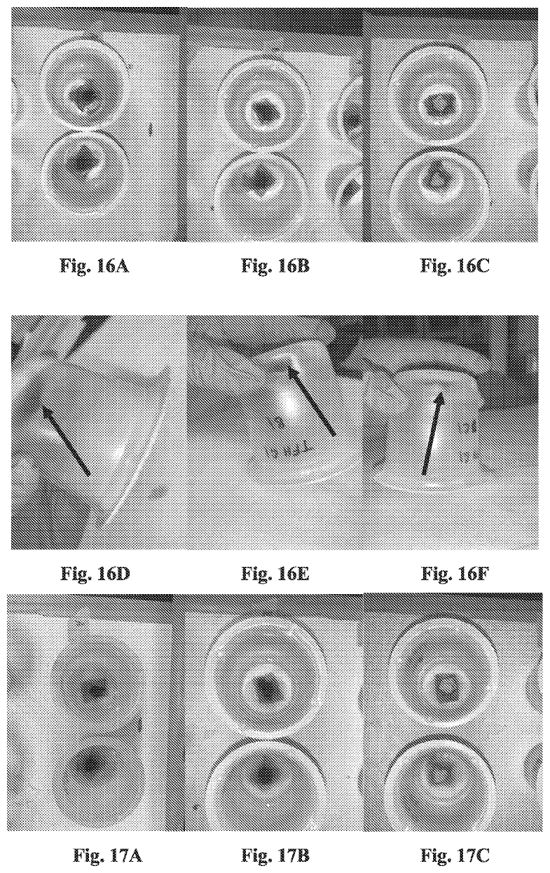

Please see FIG. 16 for images of chitosan flowable dressings (FIG. 16A Type Z; FIG. 16B 1 to 1; FIG. 16C CsGelatin2) adhered to GI tissue sitting in horizontal plane and the same dressings (FIG. 16D Type Z; FIG. 16E 1 to 1; FIG. 16F CsGelatin2) turned upside down.

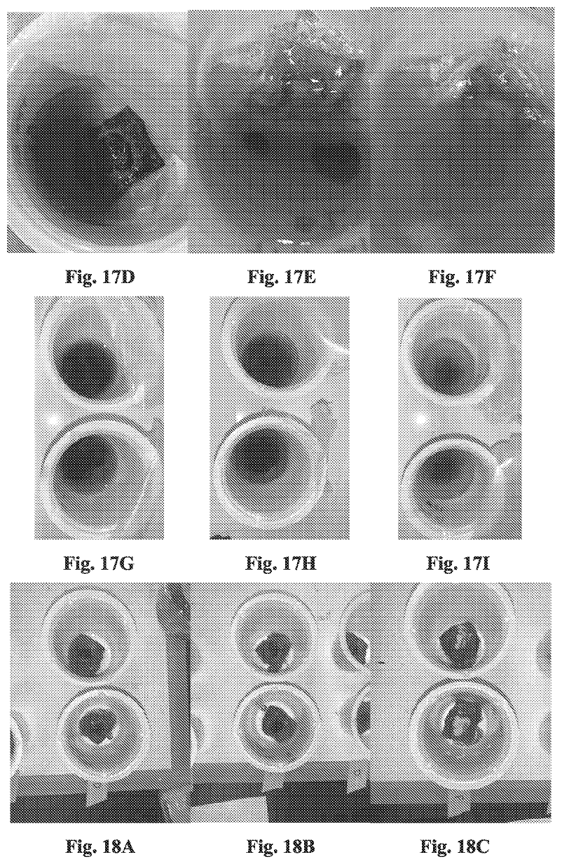

Please see FIG. 17 for images of chitosan flowable dressings (FIG. 17A Type Z; FIG. 17B 1 to 1; FIG. 17C CsGelatin2) adhered to GI tissue during ex-vivo testing at time=0; the same dressings (FIG. 17D Type Z; FIG. 17E 1 to 1; FIG. 17F CsGelatin2) at 7.8 hours and the dressings (FIG. 17G Type Z; FIG. 17H 1 to 1; FIG. 17I CsGelatin2) at 19.9 hrs.

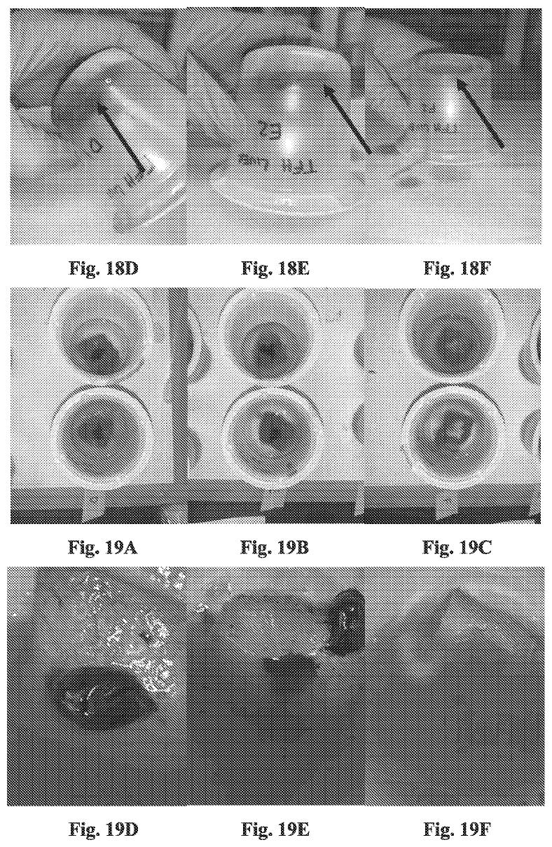

Please see FIG. 18 for images of chitosan flowable dressings (FIG. 18A Type Z; FIG. 18B 1 to 1; FIG. 18C CsGelatin2) adhered to Liver tissue sitting in horizontal plane and the same dressings (FIG. 18D Type Z; FIG. 18E 1 to 1; FIG. 18F CsGelatin2) turned upside down.

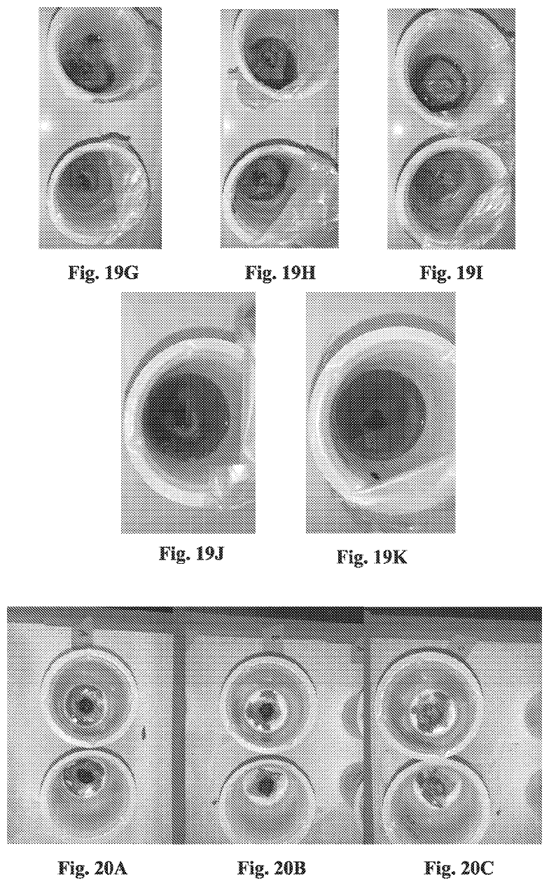

Please see FIG. 19 for images of chitosan flowable dressings (FIG. 19A Type Z; FIG. 19B 1 to 1; FIG. 19C CsGelatin2) adhered to Liver tissue during ex-vivo testing at time=0; the same dressings (FIG. 19D Type Z; FIG. 19E 1 to 1; FIG. 19F CsGelatin2) at 7.8 hours; the dressings (FIG. 19G Type Z; FIG. 19H 1 to 1; FIG. 19I CsGelatin2) at 19.9 hrs; and the remaining dressings ((FIG. 19J Type Z; FIG. 19K 1 to 1.) adhered at 33 hours.

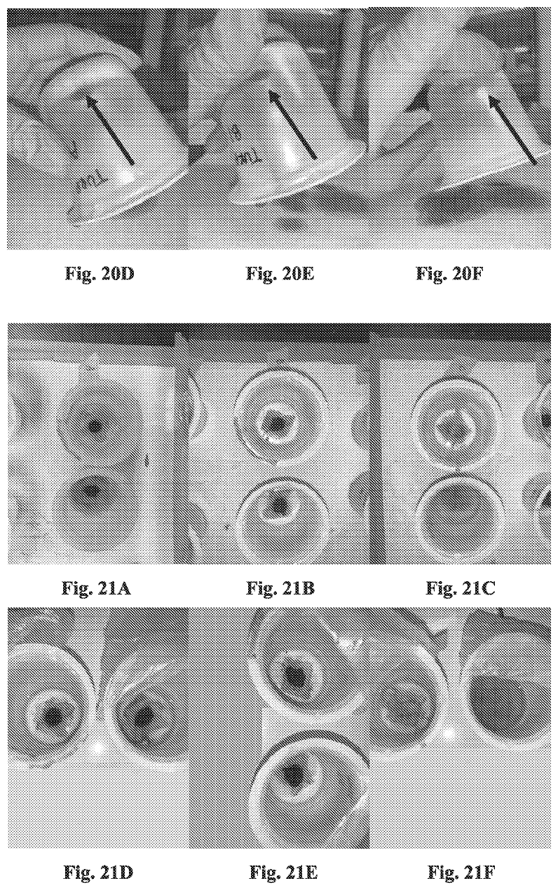

Please see FIG. 20 for images of chitosan flowable dressings (FIG. 20A Type Z; FIG. 20B 1 to 1; FIG. 20C CsGelatin2) adhered to TURP Bladder tissue sitting in horizontal plane and the same dressings (FIG. 20D Type Z; FIG. 20E 1 to 1; FIG. 20F CsGelatin2) turned upside down.

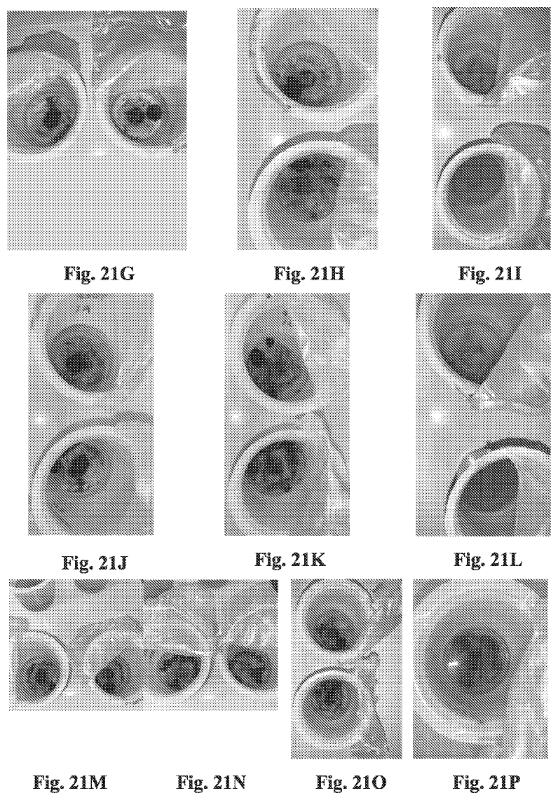

Please see FIG. 21 for images of chitosan flowable dressings (FIG. 21A Type Z; FIG. 21B 1 to 1; FIG. 21C CsGelatin2) adhered to TURP Bladder tissue during ex-vivo testing at time=0; the same dressings (FIG. 21D Type Z; FIG. 21E 1 to 1; FIG. 21F CsGelatin2) at 2.25 hours; the dressings (FIG. 21G Type Z; FIG. 21H 1 to 1; FIG. 21I CsGelatin2) at 6.2 hrs; the dressings (FIG. 21J Type Z; FIG. 21K 1 to 1; FIG. 21L CsGelatin2) at 13.9 hours; the dressings (FIG. 21M Type Z; FIG. 21N 1 to 1.) at 24 hours; the dressings (FIG. 21O Type Z; FIG. 21P 1 to 1.) at 29.5.7 hours; and at 48.7 hours.

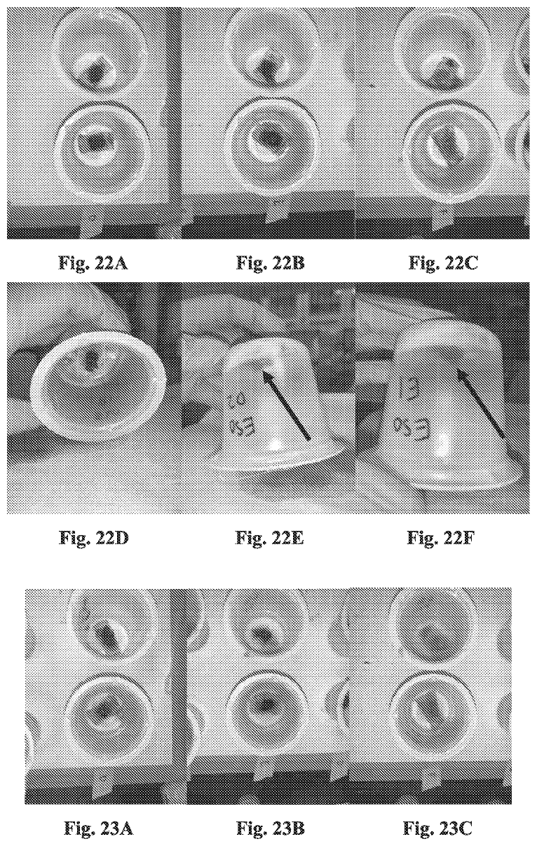

Please see FIG. 22 for images of chitosan flowable dressings (FIG. 22A Type Z; FIG. 22B 1 to 1; FIG. 22C CsGelatin2) adhered to esophageal tissue sitting in horizontal plane and the same dressings (FIG. 22D Type Z; FIG. 22E 1 to 1; FIG. 22F CsGelatin2) turned upside down.

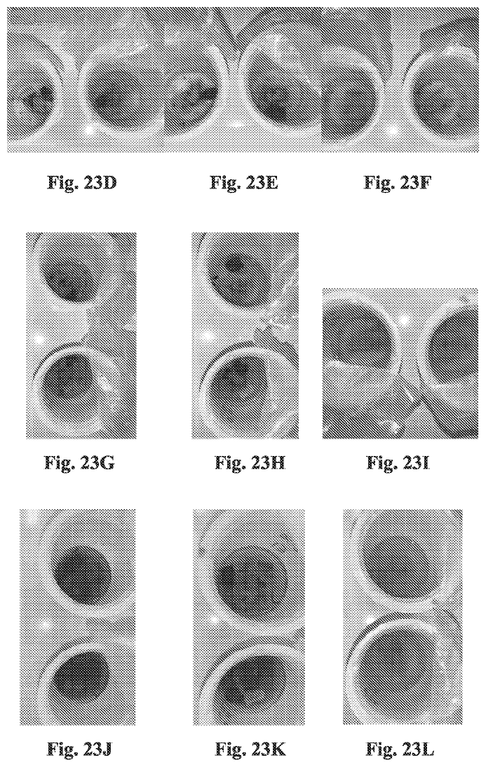

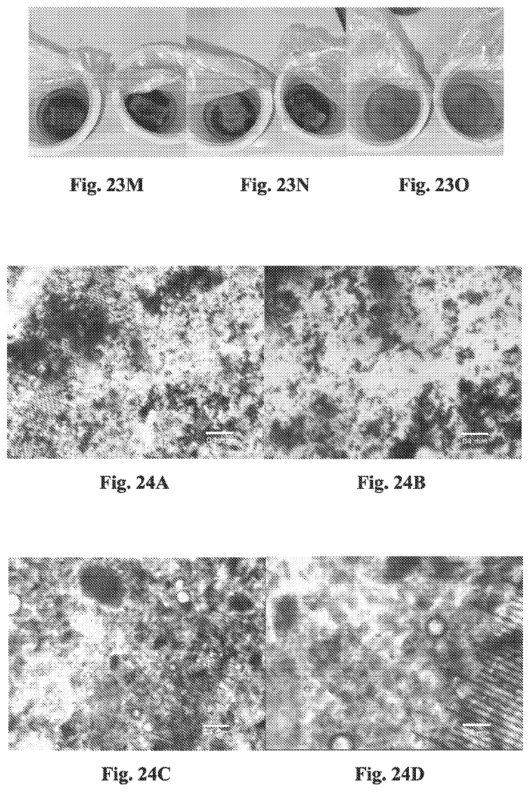

Please see FIG. 23 for images of chitosan flowable dressings (FIG. 23A Type Z; FIG. 23B 1 to 1; FIG. 23C CsGelatin2) adhered to esophageal tissue during ex-vivo testing at time=0; the same dressings (FIG. 23D Type Z; FIG. 23E 1 to 1; FIG. 23F CsGelatin2) at 2.25 hours; the dressings (FIG. 23G Type Z; FIG. 23H 1 to 1; FIG. 23I CsGelatin2) at 6.2 hrs; the dressings (FIG. 23J Type Z; FIG. 23K 1 to 1; FIG. 23L CsGelatin2) at 13.9 hours; the dressings (FIG. 23M Type Z; FIG. 23N 1 to 1; FIG. 23AO CsGelatin2) at 24 hours.

### Conclusions

The catechol modified chitosan flowable dressings of the invention demonstrates persistence as an adhered mound and protective material on tissue in difficult wet conditions beyond 12 hours, and significantly greater persistence as an adhered mound and material layer compared to FLOSEAL. The wet conditions of testing mimic clinical use in wet natural orifice minimally invasive applications such as in the gastrointestinal tract, the urethra and the bladder. The flowable chitosan material of the invention remains substantially adhered to tissue under difficult wet conditions for up to 49 hours substantially as a protective layer and then after 49 hours substantially degrades and dissolves to be removed from the body by excretion.

### Example 6

### Light Microscope Images of the Flowable Dressing Powder and its Wetting Behavior

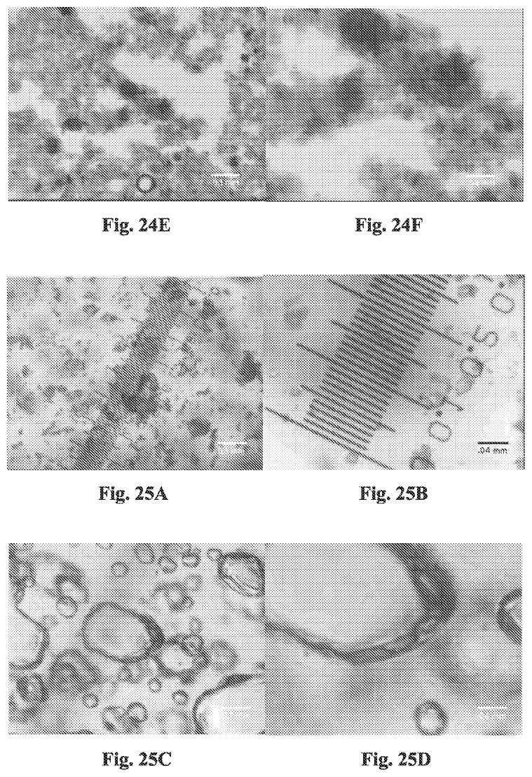

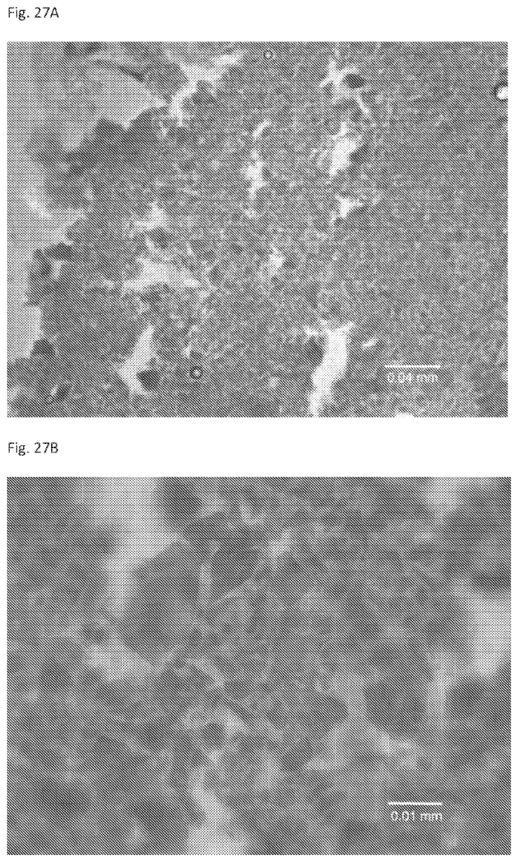

Images of powder compositions of the invention at near magnifications of 40×, 100× and 400× (further 1.75× on printing) were collected using an Amscope T490-DK optical trinocular microscope. Please note that FIGS. 24, 25 and 26 are shown at 40× and 100× magnification while FIG. 27 is shown at 100× and 400× magnification.

Please see FIG. 24 for images of Type Z powder (FIG. 24A and FIG. 24B: dry; FIG. 24AC and FIG. 24D: wetted with water; FIG. 24E and FIG. 24F: wetted with blood and water).

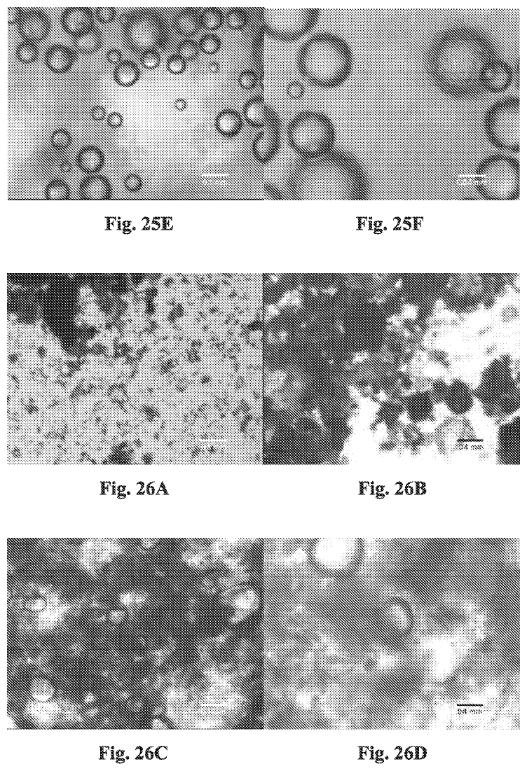

Please see FIG. 25 for images of CsGelatin2 (FIG. 25A and FIG. 25B: dry; FIG. 25C and FIG. 25D: wetted with water; FIG. 25E and FIG. 25F: wetted with blood and water).

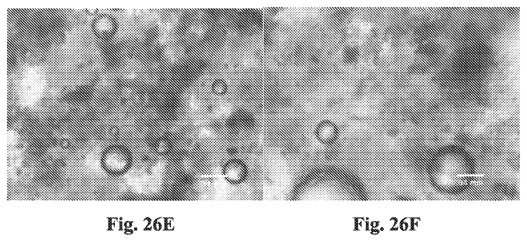

Please see FIG. 26 for images of 1:1 Type Z and CsGelatin2 (FIG. 26A and FIG. 26B: dry; FIG. 26C and FIG. 26D: wetted with water; FIG. 26E and FIG. 26F: wetted with blood and water).

Please see FIG. 27 for images of Type Y powder wetted with blood and water (FIG. 27A and FIG. 27B:).

### Conclusions

The light microscopy images 24 thru 27 demonstrate the particulate micron sized particle composition of the chitosan flowable dressing remains particulate on wetting with water and biological fluids such as blood. Also, light microscope examination of blood flow through the deposited wet flowable matrix demonstrates a rapid and penetrating procession of red blood cells and platelets through the porous particulate matrix and the subsequent binding of the same red blood cells and platelets with attachment to a suspended particle surface after navigating along the interconnected porous channels.

### Example 7

### FLOSEAL Control Test Images

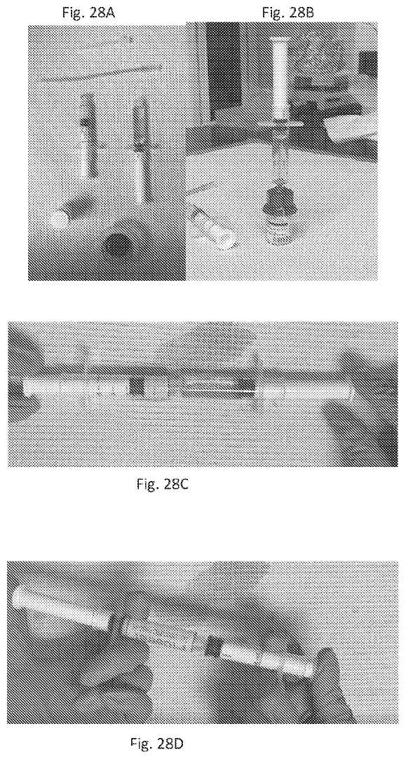

Please see FIGS. 28A-28D for images of FLOSEAL preparation.

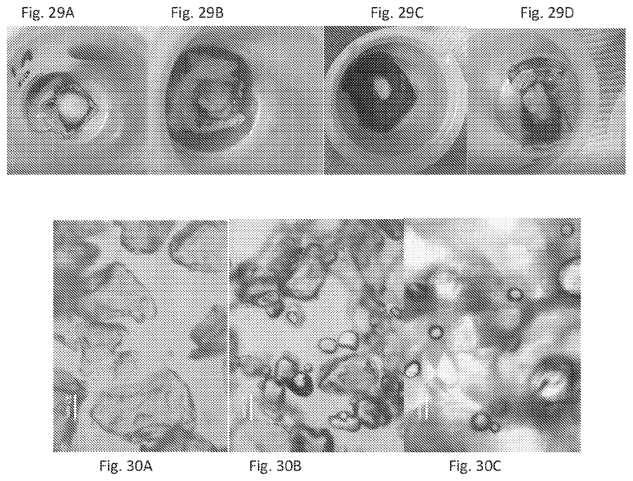

Please see FIGS. 29A-29D for images of FLOSEAL on stomach, bladder, liver and esophagus.

Please see FIG. 30 for images of FLOSEAL powder using an Amscope T490-DK microscope (FIG. 30A dry; FIG. 30B wetted with water; FIG. 30C wetted with blood).

### Example 8

### Ex-Vivo Testing of Flowable Chitosan Type Y and Type Y2 Dressings with Hemospray Powder Control

Type Y and Type Y2 catechol modified chitosan flowable dressings were tested against HEMOSPRAY powder control (Lot No. W4529951) in benchtop beaker test ex-vivo studies similar to those described in Example 5. There were two differences in the beaker test method described here compared to Example 5: i) the beaker studies of Y & Y2 flowable dressings and Hemospray powder included use of an IKA KS 260 shaker shaking at 60 rpm inside the 37° C. controlled environment; ii) small 0.5″×0.5″ holes/craters were made in the test tissues to better mimic the environment of lesions such as peptic ulcers. The tissues tested during these studies were sourced fresh from porcine animal harvest of stomach mucosal tissue, and bladder. The testing was performed at 37° C. fully immersed in wet environments of i) synthetic gastric fluid for the stomach tissue (Type Y, Type Y2, and HEMOSPRAY); and ii) synthetic urine for the bladder tissue (Type Y only).

TABLE 10
Chitosan Flowable Hemostatic Dressing Formulations

| | | | Powder:Water | H2O added |
|---|---|---|---|---|
| Formulation | Label/Test | Mass (g) | (m/v) | (ml) |
| Type Y | Y-GI | 0.400 | 1:9 | 3.6 |
| Type Y2 | YI-GI | 0.400 | 1:20 | 8.0 |
| Type Y | Y-TURP | 0.400 | 1:8.5 | 3.4 |
| HEMOSPRAY | H-GI | 0.15 | NA | NA |

### Methods

### Preparation of Tissues

The stomach and bladder tissue were cut into ~1.5×1.5″ pieces, without removing the lining. A concave surface was created in the test tissue sample surfaces by lifting the mucosa and cutting a small wound hole (about 0.5″×0.5″) in the middle to mimic ulcer site.

The base of tissue was fixed with cyanoacrylate cement to the bottom of a 150 mL polystyrene beaker cup. The top surface of the adhered tissue was wet with one to two drops of test system solution (synthetic urine, synthetic gastrointestinal fluid) and then 2-3 drops of bovine blood added to the wound hole.

The control HEMOSPRAY, 0.6 g, was scooped into a powder puffer and puffed onto the stomach and blood. Four replicates were tested each of 0.6 g of HEMOSPRAY.

The chitosan flowable dressings were prepared as follows: One 10 ml syringe contains the powder components. A corresponding male luer lock syringe was filled with the volume of water listed in Table 10. The syringes were connected and mixed by alternate plunging of the syringe plungers at close to 1 plunge per second for 30 seconds. Once mixed, all the flowable dressing was pushed into one syringe and connected to a 220 mm×2.7 mm (ID 2.1 mm) catheter. The flowable dressing was primed into the catheter so the flowable dressing reached near the distal end of the catheter. One syringe with ~0.4 g of dry powder was used to prepare a test replicate pair.

The flowable dressings and HEMOSPRAY powder were delivered to their pre-wet tissue test surfaces to sit for 30 minutes before test solution systems were added to the polystyrene beaker to fully submerge the flowable dressings in synthetic gastrointestinal fluid or synthetic urine.

The test beakers with their tissue, test flowable dressings and control HEMOSPRAY were placed on the shelves inside a 37° C. incubator and IKA KS 260 shaker and allowed to shake at 60 rpm with periodic observation of tissue adhesion and resistance of the flowable composition to the fully submersed wet environment.

The chitosan flowable test and HEMOSPRAY control samples were monitored for detachment time (hours) of bulk (mound) sample material from tissue. The time of observation of bulk detachment was recorded with time provided as the mean time between when the flowable dressing mound was found to be detached and the last time it was seen to be attached.

The chitosan flowable dressing test and HEMOSPRAY control samples were also monitored for the time at which no bound surface layer could be observed on the tissue surface. Typically, the bulk of flowable hemostat detaches leaving a layer of adhered sample on the tissue surface. This adhered layer on the tissue is desirable to help prevent rebleeding, and also to aid in tissue healing in the case of the chitosan catechol material. The time of disappearance of surface residue is described here as time for absence of adhered sample material on tissue and is recorded with time provided as the mean time between when adhered dressing layer is found to be substantially absent and the last time it was seen to be attached.

### Results

Results of the ex-vivo tissue adherence testing are provided in Tables 7 to 9 above.

TABLE 11
Average Detachment Time (hours) of bulk
(mound) sample material from Tissue

| Formulation | Stomach/GI | TURP |
|---|---|---|
| Type Y | 30.02 ± 14.17 | 31.05 ± 21.14 |
| Type Y2 | 14.30 ± 0.0 | NA |
| Hemospray | 10.36 ± 6.09 | NA |

TABLE 12
Average Time (hours) for absence of
adhered sample material on Tissue

| Formulation | GI | TURP |
|---|---|---|
| Type Y | 32.08 ± 16.25 | 37.7 ± 11.74 |
| Type Y2 | 14.30 ± 0.0 | NA |
| Hemospray | 13.88 ± 0.00 | NA |

### Conclusions

The catechol modified chitosan flowable dressings of the invention demonstrates persistence as an adhered mound and protective material on tissue in difficult wet conditions beyond 12 hours, and significantly greater persistence as an adhered mound and material layer compared to HEMOSPRAY. The wet conditions of testing mimic clinical use in wet natural orifice minimally invasive applications such as in the gastrointestinal tract, the urethra and the bladder.

### Example 9

### Acute In Vivo Porcine Hepatic Capsular Stripping Model of Hemostasis

Acute in vivo testing was performed in a domestic female porcine, body weight 40-50 Kg. and treatment was with a 1:1 Type Z CsGelatin2 CEHFD prototype. All experiments were performed in accordance with the 2011 National Research Council, “Guide for the Care and Use of Laboratory Animal” and applicable federal regulations. The protocol for the animal is in accordance with the NIH Guidelines for the Care and Use of Laboratory Animals and was approved by the Institutional Animal Care and Use Committee. All procedures and care of the animals were performed at the approved animal research facility. The animal was anesthetized and a laparotomy was performed to expose the liver. To induce a state of coagulopathy, 5000 units of heparin, was given intravenously (IV). A continuous infusion of heparin of 50 units/kg was used during the procedure to maintain anticoagulation. An activated clotting time (ACT) level was tested after 10 minutes and then every 20 minutes during the procedure with additional heparin (50% of the original dose, 2500 units) given IV as needed to maintain ACT>250 seconds anticoagulation. A 2.5 cm diameter and 1 cm deep concave injury was made in the liver lobe using a rotary cutting tool. Bleed rate immediately before treatment was determined by absorbing blood flow for 15 seconds in pre-weighed gauze and determining weight change of the gauze. The test flowable hemostat volume (4 ml) was applied directly into the bleeding injury site to cover and fill the wound with a 10 ml balloon catheter applied with total load of 35 g load over the injury site for 3 minutes after which it was deflated and removed. A decision of hemostasis success or failure was made at the discretion of the surgeon applying the flowable hemostat. The bleed rate immediately after flowable dressing application and balloon catheter removal was measured.

### Results

Pre-treatment bleed rate was 12.72 g/min. Activated clotting time (ACT) was greater than 198 seconds. Surgeon rated the application as hemostatic with final bleed rate determined as <0.9 ml/min (>90% reduction in bleeding).

### Example 10

### Acute In Vivo Porcine Spleen & Hepatic Capsular Stripping Model of Hemostasis with Hemospray Control

The objective of the animal experiment was to evaluate 4 different catechol chitosan flowable dressings of Types Z, Y, Y1 and Y1_{resid} (Y1_{resid}=residual fine particles 12.5-31.5 microns radius of gyration removed by sieving from Y1) against positive control HEMOSPRAY (Lot No. W4337018) hemostatic powder for control of difficult bleeding. The acute bleeding control testing was performed in heparinized porcine injuries of liver and spleen parenchyma.

All experiments were performed in accordance with the 2011 National Research Council, “Guide for the Care and Use of Laboratory Animal” and applicable federal regulations. The protocol for the animal is in accordance with the NIH Guidelines for the Care and Use of Laboratory Animals and was approved by the Institutional Animal Care and Use Committee. All procedures and care of the animals were performed at the approved animal research facility.

### Methods

Hemostatic efficacy was evaluated by visual score (or rank) assessment of bleeding around the application using the bleeding score 0 to 5 from “Comparison of Two Gelatin and Thrombin Combination Hemostats in a Porcine Liver Abrasion Model” by K. M. Lewis et al. 2013 [26]. Low bleeding scores (0-2) indicate control of bleeding (hemostatic success) while higher scores (3-5) indicate little to no control of bleeding (hemostatic failure). The scoring system is summarized i-v below:
i. 0=no bleeding no nothing
ii. 1-2=no active bleeding, perhaps some left over blood
iii. 3=slow, active bleeding
iv. 4=fast, active bleeding
v. 5=out of control bleeding

Treated injury hemostatic score was evaluated at 1.5 minutes and 3 minutes for the parenchymal injuries with an observation time of 3 minutes after the final application to confirm success or failure. The decision of bleed score was made at the discretion of the surgeon. Although the study was open label, the surgeon was blinded to which catechol chitosan test article was being applied. All catechol modified chitosan dressings of the experiment were prepared at room temperature with solid powder: water ratio (g/ml) in the range of 1:7.0-10.0 which was mixed together from male/female Luer connected 10 ml syringes with delivery from the male Luer syringe with catechol modified chitosan dressing volume close to 4 ml.

Heparinized porcine parenchymal capsular injuries of the liver and spleen were prepared using surgical scissors, tweezers and 6 mm diameter biopsy punch (6 mm diameter×3 mm deep injuries). The parenchymal injury model provides a standard model of difficult to control anticoagulated bleeding with sufficient replicates to investigate statistical significance. The catechol modified chitosan dressings were applied directly onto the bleeding injuries from their delivery syringes with 1 to 2 ml of dressing per application. After the first 1.5 minutes, an additional was allowed if the bleed rank was above 2. HEMOSPRAY (0.1 to 0.2 g) application was performed by pouring the HEMOSPRAY centrally over a bleeding injury from a weigh boat sufficient to fill and cover the injury. No test nor control applications received tamponade. The catechol chitosan dressings and HEMOSPRAY were applied as randomized pairs in the parenchymal injury testing. Two animals were tested over two days with a total of 2×27 injury applications over both days.

### Results

Mean activated clotting time (ACT) for the parenchymal injuries was 525 seconds and the mean pre-treatment bleeding rate was 3.97 g/min, with no significant differences between HEMOSPRAY and the catechol modified chitosan dressings.

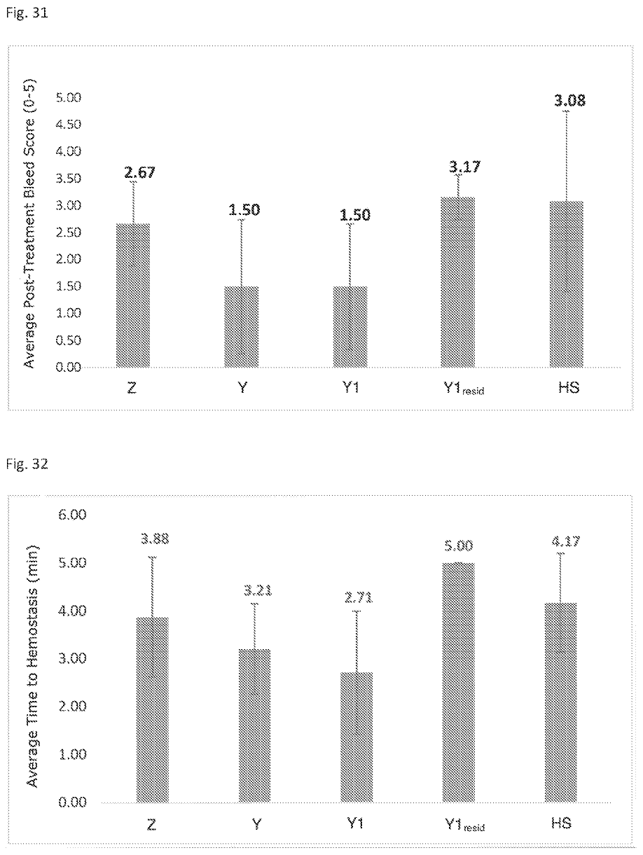

The post-treatment results of the in vivo injury testing of the 4 catechol modified chitosan test materials (Z, Y, Y1 and Y1_{resid}) and the control HEMOSPRAY material are provided in Tables 13 and 14. FIGS. 31 and 32 provide the average post-treatment bleed scores and average time to hemostasis respectively for the four test materials and HEMOSPRAY control. The catechol chitosan flowable hemostatic dressings Y and Y1 demonstrated statistically equivalent final bleed scores following the total 3-minute observation time, with both Y and Y1 demonstrating significantly (see Table 14) lower final bleed scores compared to the control HEMOSPRAY (HS). Y and Y1 demonstrated final average bleed scores of 1.5, while HEMOSPRAY had a final average score of 3.08 (p<0.05).

The least efficacious hemostatic performance of the fine particles (sieved to be 25-63 microns) of Y1_{resid} and the most efficacious hemostatic performance of Y1 (with identical composition to Y1_{resid} but fine particles removed) demonstrates the criticality of flowable matrix pore size in flowable matrix composition. There is little to no appreciation of this in the scientific and patent literature. The substantially similar hemostatic performance between Y and Y1 (average post-treatment bleed scores of 1.50 and 1.50; and average times to hemostasis of 3.21±0.94 and 2.71±1.29 respectively) indicates that the sieving removal of 25 micron particles (with radius of gyration of 12.5 microns) provides sufficient porosity size to enable close to optimal hemostatic performance.

TABLE 13
Post-Treatment Hemostatic Testing Results

| Samples | Z | Y | Y1 | Y1_{resid} | HS |
|---|---|---|---|---|---|
| Number of | 12 | 12 | 12 | 6 | 12 |
| Applications | | | | | |
| Average Final Bleed | 2.67 ± | 1.50 ± | 1.50 ± | 3.17 ± | 3.08 ± |
| Score (0-5) | 0.78 | 1.24 | 1.17 | 0.41 | 1.68 |
| Average Time to | 3.88 ± | 3.21 ± | 2.71 ± | 5.00 ± | 4.17 ± |
| Hemostasis (min) | 1.25 | 0.94 | 1.29 | 0.00 | 1.03 |
| Average Bleed Rate | 5.87 ± | 4.09 ± | 4.17 ± | 1.77 ± | 2.87 ± |
| (g/min) | 2.86 | 1.04 | 1.64 | 1.04 | 2.65 |

TABLE 14
Hemostatic Testing Statistical Metrics

| | HS vs. Y | HS vs. Y1 | Y vs. Y1 | Test Method |
|---|---|---|---|---|
| | p-value | p-value | p-value | (α = 0.05) |
| Average Final | 0.024 | 0.028 | 0.862 | Mann-Whitney |
| Bleed Score | | | | Wilcoxon |
| (0-5) | | | | Analysis |
| Time to Hemostasis | 0.026 | 0.006 | 0.289 | 2-tailed t-test |
| (min) | | | | |
| Average Bleed Rate | 0.158 | 0.166 | 0.895 | 2-tailed t-test |
| (g/min) | | | | |

### Conclusions

The catechol modified chitosan flowable dressings of the invention may be delivered accurately and with minimum preparation to difficult to access anticoagulated bleeding injuries to control the bleeding rapidly and effectively. The porosity of the flowable dressing matrix is a critical factor of its hemostatic efficacy. In porcine parenchymal injury testing, catechol modified chitosan dressings were found to be superior to HEMOSPRAY in rapid and effective control of anticoagulated bleeding.

### Example 11

### Acute In Vivo Porcine Forrest 1A Gastroepiploic Arterial Injury Model Testing

The objective of the animal experiment was to evaluate catechol chitosan flowable dressings of Types Y and Y1 against positive control HEMOSPRAY (Lot No. W4337018) hemostatic powder for control of gastrointestinal hemorrhagic Forrest 1a bleeding. The acute bleeding control testing was performed in heparinized porcine lacerations of the gastroepiploic bundle placed inside the stomach [27, 28].

All experiments were performed in accordance with the 2011 National Research Council, “Guide for the Care and Use of Laboratory Animal” and applicable federal regulations. The protocol for the animal is in accordance with the NIH Guidelines for the Care and Use of Laboratory Animals and was approved by the Institutional Animal Care and Use Committee. All procedures and care of the animals were performed at the approved animal research facility.

### Methods

Hemostatic efficacy was evaluated by determining time to hemostasis following full syringe delivery of catechol modified chitosan dressings. The catechol modified chitosan flowable dressing time to hemostasis was compared against time to hemostasis of delivered HEMOSPRAY control powder poured generously (0.6 g) over a similarly bleeding Forrest 1a bleeding injury. Pre-treatment bleed rate was measured by weight of blood absorbed in a folded 2×2 surgical gauze dressing held for 15 seconds against the injury site. Activated clotting time during the testing was determined as >250 seconds.

The treated injuries were evaluated at 2.5 minute intervals with further application of hemostatic treatment if bleeding remained uncontrolled. A decision of hemostasis success was made at the discretion of the surgeon. Although the study was open label, the surgeon was blinded to which catechol chitosan test article was being applied. All catechol modified chitosan dressings of the experiment were prepared at room temperature with solid powder:water ratio (g/ml) in the range of 1:7.0-10.0 which was mixed together from male/female Luer connected 10 ml or 20 ml syringes with delivery from a male Luer syringe containing the catechol modified chitosan dressing volume close to 4 ml. The catechol modified chitosan samples were delivered via a pre-warmed 220 mm×2.7 mm OD×2.1 mm ID catheter (1 minutes at 37° C.), and backfilled with saline using a balloon inflation device to allow delivery of the greater part of the dressing in the catheter. HEMOSPRAY powder was delivered dry from a modified transfer pipette. A maximum two lacerations were made to each bundle. Injuries were not randomized. The applications were used to investigate hemostatic efficacy, time to hemostasis and dressing deliverability. No applications received tamponade and wound sites were re-used by removing prior applications with 0.9% isotonic saline solution lavage and removing any clots present in the injury site with forceps.

The porcine gastroepiploic arterial bundle injury model [27, 28] is an accepted model of clinically relevant upper gastrointestinal Forrest-1a hemorrhage. The catechol modified chitosan dressings were applied directly onto the bleeding injuries from their delivery syringes with 3 to 4 ml of dressing per application. After the first 2.5 minutes, additional applications of hemostatic agent were allowed for up to 10 minutes after the first application. HEMOSPRAY (0.6 g) application was performed by pouring the HEMOSPRAY centrally over a bleeding injury from a dropping pippette. No sample applications received tamponade. The catechol chitosan dressings and HEMOSPRAY were applied as randomized pairs in the testing. Two animals were tested over two days with a total of 2×3 injury applications over both days.

### Results

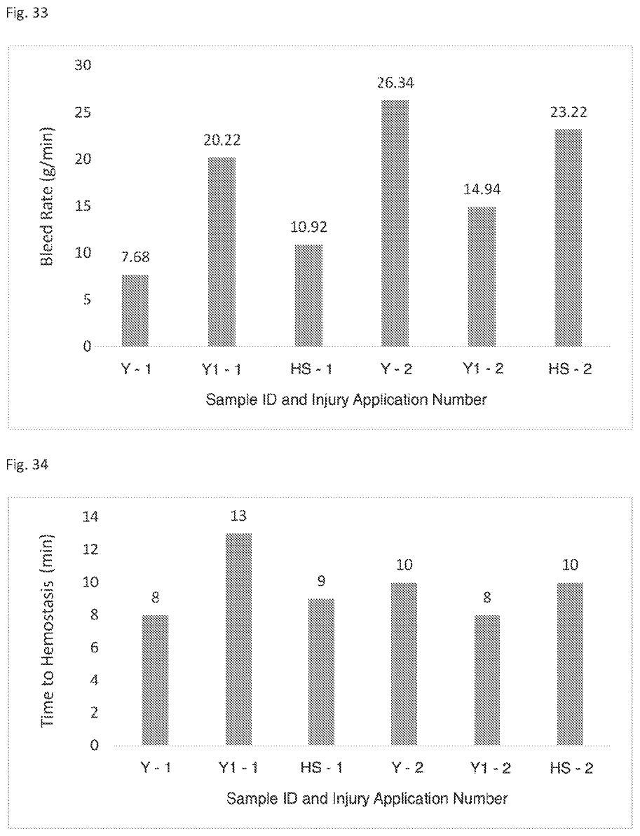

It was found that deliveries of the catechol chitosan Y & Y1 dressings and HEMOSPRAY to the hemorrhagic gastroepiploic bleeding were able to control Forrest 1a hemorrhage 100% effectively within 15 minutes without any recurrence of bleeding after achieving successful hemostasis. The average times to hemostasis for catechol modified dressings (Y & Y1) and HEMOSPRAY were 9.75 and 9.5 minutes respectively. FIG. 33 shows a histogram box plot of pre-treatment bleed rate for catechol modified chitosan (Y & Y1) and HEMOSPRAY (HS) applications. FIG. 34 shows a histogram box plot of time to hemostasis for treatment of gastroepiploic arterial injuries by applications of catechol modified chitosan dressings (Y & Y1) and control HEMOSPRAY (HS).

One of the C1 applications used 2 syringe doses, while the other used just one. Both C2 applications used only one syringe dose. HEMOSPRAY had one application that used just one 0.6 g dose, and another that used 1.2 g during the first dose and then another 0.6 g dose after some observation time. On delivery to the injury site, the Y & Y1 applications adhered immediately to the injury surface and resisted the spurting blood flow. The delivery of the Y flowable dressing demonstrated a formed rod or “noodle” appearance at the beginning of the delivery which disappeared over the course of the delivery. Flowable dressing Y1 also demonstrated the appearance of a formed or otherwise extruded rod which disappeared faster than in the C1 prototype delivery. Both Y and Y1 dressing deliveries were similar in the occurrence of formed rod on delivery and in their extent of mixing with the back-fill saline in the catheter. None of the applications received any tamponade, including HEMOSPRAY. The delivery of the HEMOSPRAY powder, by pouring/puffing, was accurate and local unlike the dispersive powder stream delivery of the HEMOSPRAY gas spray delivery device.

### Conclusion

The catechol modified flowable dressing system and its delivery are accurate and effective in controlling anti-coagulated, gastrointestinal hemorrhage. The catechol modified chitosan flowable dressings of the invention disclosure are non-inferior to FDA cleared HEMOSPRAY powder for the control of gastrointestinal bleeding.

### Example 12

### Human Epidermis Equivalent (HEE) Testing to Assess Irritation

Human epidermis equivalent (HEE) testing was performed to assess irritation (iFyber LLC, Ithaca, NY). The test samples were catechol modified chitosan prepared as Y flowable dressing with water (0.40 g of Y powder dispersed in 5.0 ml of water) and compressed catechol modified freeze dried sheet (Y compressed sheet). The catechol modified chitosan sheet used for the milling of Y powder was uncompressed with Y sheet membrane density close to 0.005 g/cm³ (>99% void space). The Y sheet test sample (pure Y catechol modified chitosan) was tested in the irritation testing described here as Y compressed sheet to assist in maintaining sheet integrity during extraction. The Y compressed sheet close to 100 microns thickness was compressed from its uncompressed original, dry, freeze-dried low density thickness near 7 mm by compression between parallel heated platens at 60° C. to density close to 0.4 g/cm³ (<0.75% void space). The catechol modified chitosan materials were sterilized under gamma irradiation at 25-40 kGy. HEMOSPRAY (Lot No. W4337018) powder was included in the study as a positive control sample. The study was performed in accordance with ISO 10993-23 for assessing the irritation potential of medical devices.

### Methods

Human epidermis equivalent (HEE) tissues were established following according to protocol: after thawing and expanding keratinocytes (ATCC PCS-201-012), a determined density (2×10⁵ cells/insert) were seeded in 12-well cell culture inserts. The cells were cultured submerged for five days, then brought to the air/liquid interface and cultured for an additional 8 days, with media replacement every 2-3 days. Extracts of the test samples (Y flowable dressing, Y compressed sheet) and control HEMOSPRAY sample were prepared according to ISO 10993-12. Before preparing the extracts for the study, an absorption assessment was performed for each test sample type using DI water to determine the appropriate extraction volume. The chosen extraction ratios were 6 cm²/mL for the Y compressed sheet and 0.2 g/mL for the Y flowable dressing and HEMOSPRAY powder. Each sample was weighed or measured, an excess of water was added, the samples were incubated overnight at 37° C., and then the remaining water was measured. The determined volumes for achieving the extraction ratios were 1 mL for 0.2 g Y flowable dressing, 2.5 mL for 0.2 g HEMOSPRAY powder, and 3.5 mL for a 6 cm²-sized Y compressed sheet. Three replicates of each test sample were used to prepare extracts for the irritation test. The HEMOSPRAY powder was transferred to three wells of a 12-well plate at 0.2 g/well, and then placed under UV light for 30 mins to sterilize. The Y flowable dressing was prepared, then 0.2 g/well was aseptically added to the 12-well plate. A 28 mm diameter punch was used to cut uniformly sized disc pieces of the Y compressed sheet, which were placed in a 6-well plate. HEE culture medium was used as the extraction medium. This medium is typically serum-free; however, in order to extract both polar and non-polar components from the test articles, fetal bovine serum (FBS) was added at 5% v/v. The appropriate volume of medium was then added to the three replicates of each test sample. The plates were wrapped with Parafilm and incubated for 72 h in a shaking incubator at 37° C., 28 rpm. Controls, consisting of 1×PBS (negative control) and HEE culture medium with FBS (vehicle control), were incubated under the same conditions alongside the test and control samples.

At the conclusion of the extraction time point, a volume of 200 μL of extract was removed directly from each sample and immediately added to the surface of the HEE tissues. Once all tissues were treated, they were placed in a humidified incubator at 37° C., 5% CO₂. After 20 h, all HEE samples were removed from the incubator, and each sample was rinsed ×3 with sterile 1×PBS. The inserts were blotted dry and then placed in a temporary storage plate (i.e., a 12-well plate containing HEE culture media) until all samples had been washed. Next, the samples were transferred to a 12-well plate containing 600 μL of 1 mg/mL MTT in phenol red-free DMEM and incubated in a humidified incubator at 37° C., 5% CO₂ for 3 h. Subsequently, the samples were transferred to a 12-well plate containing 3 mL per well of isopropanol in order to extract the formazan (MTT reaction end product). This plate was incubated at room temperature on a shaker at 120 rpm for 2 h. Next, each cell culture insert was pierced, and the solution was pipetted up and down several times to homogenize. Three 200 μL aliquots from each well were transferred to a 96-well plate, and the absorbance at 570 nm was measured. Isopropanol was included as a blank.

Data analysis was performed by first subtracting the mean background OD of the isopropanol blanks from each OD value. The mean background corrected OD of the negative control tissues was determined, which represented 100% viability. The viability per tissue, relative to the control, was then determined, and the three replicates were averaged to determine the viability per test article. Lastly, Statistical analysis was performed with GraphPad Prism software, and data were analyzed using one-way ANOVA and Tukey's post hoc test. The significance level was set at 5%.

### Results & Discussion

This study was performed to determine the irritation potential of Y flowable dressing, Y compressed sheet and the HEMOSPRAY control. The test involved the preparation of extracts of each material, which were then added to HEE tissues. After a 20 h incubation, the test articles were removed, and the tissue viability was determined via MTT assay.

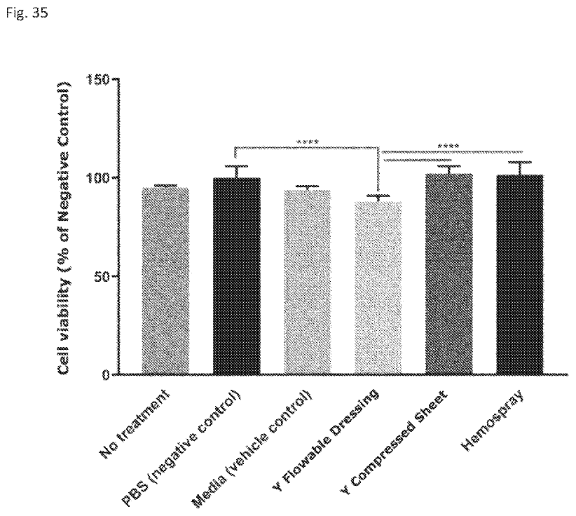

The results, depicted in FIG. 35, showed similar viability for control groups, Y flowable dressing, Y compressed sheet, and HEMOSPRAY with no significant difference (p>0.05). Y flowable dressing showed a slight reduction of cell viability relative to the negative control, Y compressed sheet, and HEMOSPRAY (p<0.05). However, the percentage of cell viability for all samples was higher than 50%.

The criteria for determination of irritation potential, adapted from ISO 10993-23, are shown in Table 15. As shown in FIG. 35, all test samples resulted in tissue viability of >50%; therefore, the catechol modified chitosan Y flowable dressing, catechol modified chitosan Y compressed sheet and HEMOSPRAY can all be classified as non-irritants, according to the findings of this study.

TABLE 15
Classification of the irritant potential of the test sample

| Criteria for in vitro interpretation | Classification |
|---|---|
| Cell viability after the exposure is <50% | Irritant |
| Cell viability after the exposure is >50% | Non-irritant |

### Conclusion

The catechol modified chitosan Y material and flowable dressing of the invention are non-irritant as per ISO-10993-23 and as such are suitable for use on mucosal tissues in natural orifice locations of the body.

### CONCLUSION

This invention disclosure demonstrates a high effective non-biologic, tissue adherent, fluid-type, flowable hemostat, for direct application to actively bleeding wounds and for prolonged application (>6 hrs) in general and difficult environments to control moderate to robust bleeding.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/386,312, filed on Dec. 6, 2022, U.S. Provisional Patent Application No. 63/386,313, filed on Dec. 6, 2022, and U.S. Provisional Patent Application No. 63/386,314, filed on Dec. 6, 2022, are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### REFERENCES

The following is a list of references listed by number and corresponding to the bracketed numbers noted throughout this specification.
1. Subramanian, D. A., R. Langer, and G. Traverso, Mucus interaction to improve gastrointestinal retention and pharmacokinetics of orally administered nano-drug delivery systems. Journal of Nanobiotechnology, 2022. 20(1): p. 362.
2. HCUP, Outcomes by 153 Gastrointestinal hemorrhage, in US Department of Health & Human/HCUPnet. 2010, US Department of Health & Human Services: Washington DC.
3. Rockey, D. C., Gastrointestinal bleeding. Gastroenterol Clin North Am, 2005. 34(4): p. 581-8.
4. Crooks, C. J., J. West, and T. R. Card, Upper gastrointestinal haemorrhage and deprivation: a nationwide cohort study of health inequality in hospital admissions. Gut, 2012. 61(4): p. 514-20.
5. Jairath, V., et al., Mortality from acute upper gastrointestinal bleeding in the United kingdom: does it display a “weekend effect”? Am J Gastroenterol, 2011. 106(9): p. 1621-8.
6. Sung, J. J., et al., Causes of mortality in patients with peptic ulcer bleeding: a prospective cohort study of 10,428 cases. Am J Gastroenterol, 2010. 105(1): p. 84-9.
7. Jairath, V., et al., Prevalence, management, and outcomes of patients with coagulopathy after acute nonvariceal upper gastrointestinal bleeding in the United Kingdom. Transfusion, 2013. 53(5): p. 1069-76.
8. Elta, G. H., Approach to the patient with gross gastrointestinal bleeding. Textbook of Gastroenterology, 2003. In: Yamada T., Alper, D. H., Editors (Lippincott Williams & Wilkins): p. 698-723.
9. Boonpongmanee, S., et al., The frequency of peptic ulcer as a cause of upper-GI bleeding is exaggerated. Gastrointest Endosc, 2004. 59(7): p. 788-94.
10. Jairath, V., M. Martel, R. F. Logan, and A. N. Barkun, Why do mortality rates for nonvariceal upper gastrointestinal bleeding differ around the world? A systematic review of cohort studies. Can J Gastroenterol, 2012. 26(8): p. 537-43.
11. Sheibani, S., et al., Natural history of acute upper GI bleeding due to tumours: short-term success and long-term recurrence with or without endoscopic therapy. Aliment Pharmacol Ther, 2013. 38(2): p. 144-50.
12. Adler, D. G., et al., ASGE guideline: The role of endoscopy in acute non-variceal upper-GI hemorrhage. Gastrointest Endosc, 2004. 60(4): p. 497-504.
13. Banerjee, S., et al., The role of endoscopy in the management of patients with peptic ulcer disease. Gastrointest Endosc, 2010. 71(4): p. 663-8.
14. Peng, Y. C., et al., Factors associated with failure of initial endoscopic hemoclip hemostasis for upper gastrointestinal bleeding. J Clin Gastroenterol, 2006. 40(1): p. 25-8.
15. Peng, Y. C., et al., Factors contributing to the failure of argon plasma coagulation hemostasis in patients with nonvariceal upper gastrointestinal tract bleeding. Hepatogastroenterology, 2010. 57(101): p. 781-6.
16. Halkerston, K., et al., PWE-046 Early Clinical Experience of Endoclot™ in the Treatment of Acute Gastro-Intestinal Bleeding. Gut, 2013. 62 (Suppl 1): p. A149.
17. Chahal, D., J. G. H. Lee, N. Ali-Mohamad, and F. Donnellan, High rate of re-bleeding after application of Hemospray for upper and lower gastrointestinal bleeds. Digestive and Liver Disease, 2020. 52(7): p. 768-772.
18. Lee, D. H. and W. Ko, Hemostatic materials in non-variceal upper gastrointestinal hemorrhage. International Journal of Gastrointestinal Intervention, 2020. 9(1): p. 1-3.
19. Mullady, D. K., A. Y. Wang, and K. A. Waschke, AGA Clinical Practice Update on Endoscopic Therapies for Non-Variceal Upper Gastrointestinal Bleeding: Expert Review. Gastroenterology, 2020. 159(3): p. 1120-1128.
20. Ofosu, A., et al., The Efficacy and Safety of Hemospray for the Management of Gastrointestinal Bleeding: A Systematic Review and Meta-Analysis. Journal of Clinical Gastroenterology, 2021. 55(5).
21. Yii, R. S. L., et al., Retained Endoscope: An Unexpected but Serious Complication of Hemospray®. Digestive Diseases and Sciences, 2022. 67(1): p. 344-347.
22. Park, J.-S., et al., Novel hemostatic adhesive powder for nonvariceal upper gastrointestinal bleeding. Endoscopy International, 2019. 7(12): p. 1763-1767.
23 Shin, J., et al., Efficacy of a novel hemostatic adhesive powder in patients with upper gastrointestinal tumor bleeding. BMC Gastroenterology, 2021. 21(1): p. 40.
24. Roberts, G. A. F., Chitin Chemistry. 1992, London: MacMillan. 9.
25. Roberts, G. A. F., Chitin Chemistry. 1992, London: MacMillan. 203-205.
26. Lewis, K. M., et al., Comparison of Two Gelatin and Thrombin Combination Hemostats in a Porcine Liver Abrasion Model. Journal of Investigative Surgery, 2013. 26(3): p. 141-148.
27. Giday, S., et al., Long-term randomized controlled trial of a novel nanopowder hemostatic agent (TC-325) for control of severe arterial upper gastrointestinal bleeding in a porcine model. Endoscopy, 2011. 43(04): p. 296-299.
28. Giday, S., et al., Safety Analysis of a Hemostatic Powder in a Porcine Model of Acute Severe Gastric Bleeding. Digestive Diseases and Sciences, 2013. 58(12): p. 3422-3428.

1. A flowable hemostatic composition comprising a particulate chitosan material and a diluent carrier liquid.

2. The flowable composition of claim 1, wherein the particulate chitosan material comprises an amount of greater than or equal to about 5% of the total weight of the flowable composition.

3. The flowable composition of claim 1, wherein the particulate chitosan material is provided as a solid or semi-solid and, optionally, wherein the semi-solid particulate chitosan material is swollen.

4. (canceled)

5. The flowable composition of claim 1, wherein the particulate chitosan material is provided as a powder, a granule, particle, fiber, or any combination thereof and, optionally, wherein: (i) the particulate chitosan material comprises regular or irregular shaped particles with radius of gyration in the range of about 10 to 350 micrometers; or (ii) the particulate chitosan material comprises one or more of a densified chitosan material, a freeze-phase-separated and dried chitosan material, a densified freeze-phase-separated and dried chitosan material, a spray-dried chitosan material, a dried cast film chitosan material, a sublimated freeze separated chitosan material, a dried freeze thawed chitosan material, and a dried asymmetrical centrifugally mixed composition; or (iii) further characterized by presentation of a specific surface area greater than about 100 cm² per gram of flowable composition.

6. (canceled)

7. The flowable composition of claim 1, wherein the particulate chitosan material comprises one or both of catechol modified chitosan and chitosan gelatin crosslinked.

8. (canceled)

9. The flowable composition of claim 1, wherein the diluent carrier liquid is one or more of water, standard 0.9% aqueous saline solution, and autologous plasma; or wherein the diluent carrier liquid comprises at least one of about 85% of the total weight of the flowable composition, about 90% of the total weight of the flowable composition, or about 95% of the total weight of the flowable composition; or wherein the diluent carrier liquid is a thermoresponsive fluid capable of delivery through a 23-gauge needle or a 24-gauge needle at about 18-25° C. and capable of gelation at about 37° C.

10-12. (canceled)

13. The flowable composition of claim 1, wherein the flowable composition is resistant to dissolution and, optionally, wherein: (i) the flowable composition comprises particulate chitosan material that does not substantially dissolve and remains solid or semi-solid; or (ii) the flowable composition is capable of resisting dissolution in at least one of urine, water, saline solution, blood, or gastrointestinal (GI) fluid at about 37° C. for at least about 6 hours; or (iii) the flowable composition is capable of resisting dissolution at a gastrointestinal or endoluminal tissue site; or (iv) the flowable composition is biocompatible.

14-16. (canceled)

17. The flowable composition of claim 13, wherein the flowable composition comprises at least a first outer layer and a second tissue adherent layer, and wherein the first outer layer resists dissolution for at least about 6 hours and the second tissue adherent layer resists dissolution for at least about 12 hours.

18. The flowable composition of claim 1, wherein the flowable composition is tissue adherent and, optionally, wherein the tissue adherent flowable composition adheres on contact for a period of time that is greater than about 6 hours to at least one of mucosal tissue and a tissue injury site.

19. (canceled)

20. The flowable composition of claim 18, wherein the flowable composition may be endoscopically applied upside down under normal gravity and adhere to tissue.

21. (canceled)

22. The flowable composition of claim 1, wherein the flowable composition is capable of delivery to a tissue site via a channel having a diameter of at least one of less than about 7 mm, less than about 4.5 mm, less than about 4.0 mm, less than about 3.2 mm, less than about 2.8 mm, and about 0.5 mm.

23-24. (canceled)

25. A method of making the flowable composition of claim 1.

26-29. (canceled)

30. A method of delivering a flowable composition to a tissue site to a subject in need thereof comprising combining a particulate chitosan material and a diluent carrier liquid prior to delivery to the subject.

31. The method of claim 30, further comprising providing the particulate chitosan material and the diluent carrier liquid as separate components for combination and, optionally, wherein the separate components are sterilized separately or, optionally, wherein the diluent carrier liquid has a viscosity that is less than or equal to 3.5 and 5.5 mPa·s.

32. (canceled)

33. The method of claim 30, further comprising delivering an amount of the flowable composition sufficient to achieve hemostasis at a bleeding tissue site of the subject.

34. The method of claim 30, further comprising delivering the flowable composition to the tissue site via a channel having a diameter of at least one of less than about 7 mm, less than about 4.5 mm, less than about 4.0 mm, less than about 3.2 mm, less than about 2.8 mm, and about 0.5 mm.

35. The method of claim 30, further comprising delivering the flowable composition to the tissue site in one or more layers and, optionally, wherein the flowable composition comprises at least a first outer layer and a second tissue adherent layer, and wherein the first outer layer resists dissolution for at least about 6 hours and the second tissue adherent layer resists dissolution for at least about 12 hours.

36. (canceled)

37. The method of claim 30, further comprising adhering the flowable composition to the tissue site and, optionally, wherein the flowable composition adheres to the tissue site upon contact and for a period of time that is greater than about 6 hours, and wherein the tissue site comprises at least one of mucosal tissue and a tissue injury.

38. (canceled)

39. The method of claim 30, wherein the flowable composition may be endoscopically applied (i) upside down under normal gravity and adhere to a tissue site; or (ii) to an endoluminal tissue site; or (iii) to a gastrointestinal tissue site.

40-41. (canceled)

42. The method of claim 39, further comprising sealing of the tissue site by the gastrointestinal hemostatic flowable composition for at least six hours in an acid environment of about pH 3 and, optionally, further comprising providing for the dissolution of the gastrointestinal hemostatic flowable composition from the tissue site over a period of time less than or equal to about seven days.

43-45. (canceled)

46. The method of claim 30, further comprising applying the flowable composition using minimally invasive techniques, optionally, wherein the minimally invasive techniques provide for remote flowable composition delivery; or (ii) applying the flowable composition to do one or both of filling and closing of resections, biopsy sites, narrow recesses, and defects and openings around hemostatic clips, sutures, clamps, staples, wires and pins.

47-48. (canceled)
